(19) United States
(12) Patent Application Publication
(10) Pub. No.: US 2008/0108557 A1
(43) Pub. Date: May 8, 2008

(54) Modified Proteins

(75) Inventors: Carsten Behrens, Copenhagen N (DK); Patrick William Garibay, Holte (DK); Magali Zundel, Dyssegaard (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/664,199

(22) PCT Filed: Sep. 29, 2005
(86) PCT No.: PCT/EP05/54901

§ 371 Date: Sep. 19, 2007

(30) Foreign Application Priority Data
Sep. 29, 2004 (DK) PA 2004 01479
Jan. 18, 2005 (DK) PA 2005 00090
Feb. 4, 2005 (DK) PA 2005 00175

(51) Int. Cl. A61K 38/02; C07K 17/10; C12P 21/00

(52) U.S. Cl. 514/8; 435/68.1; 530/395

(57) ABSTRACT

A method of conjugating peptides and proteins by means of glycosyltransferase is provided.

### FIELD OF THE INVENTION

The present invention relates to the preparation of improved drugs, especially to the preparation of modified glycoproteins having improved pharmacodynamic and/or pharmacokinetic properties.

### BACKGROUND OF THE INVENTION

Proteins of biological origin hold great promise as therapeutical agents as they often possess high efficacy and high selectivity towards their natural ligands. Being of biological origin increases the likelihood that they are non-toxic and thus safer to use than conventional small molecular drugs, as the organism already posses well defined clearing mechanisms as well as metabolic pathways for their disposal. This in combination with the fact, that proteins now can be produced by recombinant DNA techniques in a variety of different expression systems, allowing for large-scale production, render proteins ideal drug candidates. However, therapeutically interesting proteins such as hormones, soluble receptors, cytokines, enzymes, etc., often have short circulation half-life in the body, generally reducing their therapeutic utility.

Therapeutic proteins are removed from circulation by a number of routes. For some pharmacologically active proteins, there are specific receptors which mediate removal from circulation. Proteins which are glycosylated may be cleared by lectin-like receptors in the liver, which exhibit specificity only for the carbohydrate portion of those molecules. Non-specific clearance by the kidney of proteins and peptides (particularly non-glycosylated proteins and peptides) below about 50 kDa has also been documented. It has been noted that asialo-glycoproteins are cleared more quickly by the liver than native glycoproteins or proteins lacking glycosylation (Bocci (1990) Advanced Drug Delivery Reviews 4: 149).

Therapeutic proteins are also cleared from circulation by the immune system in the event that they are not completely identical to autologous proteins, since even small variations in amino acid sequence or 3-dimensional structure can render a therapeutic protein immunogenic. The immune response induced by a therapeutic protein can further have various undesired effects apart from the accelerated removal from circulation: Antibodies may interfere with or block the therapeutic effect via steric hindrance of access to binding sites in the therapeutic protein, induced antibodies may cross-react with autologous proteins and thereby result in autoimmune reactions etc.

It is also of interest to modify therapeutic proteins so as to target them to certain cells, organs or tissues. Conjugation or fusion of proteins to ligand molecules that have high affinity for molecules present in specialised cells or tissues is one known way of achieving this effect. However, chemical conjugation technology often suffers the drawback that the molecules produces are heterogeneously modified, meaning that the end-product is insufficiently characterized, and fusion of therapeutic proteins requires that the targeting moiety is itself a protein.

There is therefore a general need for provision of methods of preparing modified (therapeutic) proteins which exhibit prolonged serum half-life and/or reduced immunogenicity and/or improved pharmacological properties.

It is in itself a huge task to screen a large number of modified therapeutic proteins, but since the provision of each modified protein may require specific (semi)synthesis, there is also an increasing need for “activated” therapeutic polypeptides to which one can conveniently couple numerous moieties using basically the same coupling reaction, regardless of the nature of the moiety it is desired to couple to the therapeutic polypeptide.

Khidekel et al discloses in J. Am. Chem. Soc., 125, 16162-16163, 2003 that attachment of O-GlcNAc glycosylated proteins may be labelled to easy detection by means of glycosyltransferases.

### OBJECT OF THE INVENTION

It is an object of the invention to provide means and methods which allow for a convenient synthesis or semisynthesis of therapeutic proteins, where the introduction of the modification addresses the problems discussed above.

### SUMMARY OF THE INVENTION

The present invention i.a. provides for the prolongation of the circulating half-life of soluble glycoprotein derivatives, thus reducing the quantity of injected material and frequency of injection required for maintenance of therapeutically effective levels of circulating glycoprotein for treatment or prophylaxis. The short in vivo plasma half-life of certain therapeutically active glycoproteins is undesirable due to the frequency and the amount of soluble protein which would be required in treatment or prophylaxis. The present invention provides means to prolong the circulating half-life of such glycoproteins with an effective change to the glycoprotein structure and with the substantial maintenance of biological activity.

The present invention provides for a convenient method of preparing activated analogues of glycoproteins, where an activation group is introduced at a glycosyl group in the polypeptide, thus providing for a convenient and standardized secondary coupling of moieties of interest to the therapeutic protein via the activation site.

Thus, the invention relates to a method for preparing a modified analogue P—B′-L-M of a starting molecule M′, where said modified analogue has improved pharmacologic properties compared to the starting molecule, the method comprising the consecutive steps of

a) reacting, in the presence of a glycosyltransferase, the starting molecule M′ comprising a reactive group, with a donor substance having the formula I

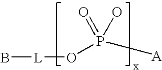

wherein

x=1 or 2,

A is selected from

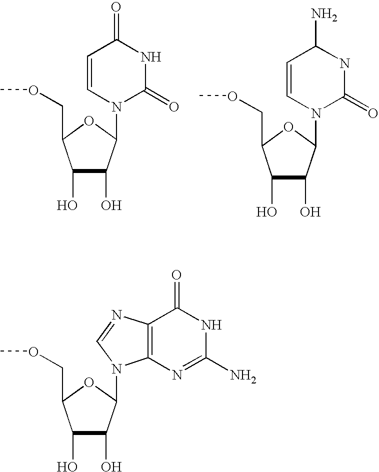

L is a divalent moiety, a bond, or a monovalent moiety L′, which comprises a protected or non-protected reactive group, which is not accessible in M′ and which specifically can react with other reactive groups, and

B is absent if L is L′ or B is a moiety which comprises a protected or non-protected reactive group, which is not accessible in M′ and which specifically can react with other reactive groups,

to yield an intermediary modified analogue of the starting molecule, said intermediary modified analogue having the formula B-L-M or L′-M, where M is M′, wherein the reactive group is absent or has been rendered substantially non-reactive,

b) if necessary, unprotecting the reactive group in B, and

c) conjugating said intermediary modified analogue to a molecule of formula P′ which comprises a reactive group not accessible in L and M and which specifically can react with B in said intermediate B-L-M to yield the modified analogue having formula P—B′-L-M, where P is P′ where the reactive group is absent or has been rendered substantially non-reactive, where B′ is a bond or B where the reactive group is absent or has been rendered substantially non-reactive, or when B is not present P′ can react with L′ in said intermediate L′-M to yield P-L-M, where L is L′ where the reactive group is absent or has been rendered substantially non-reactive.

The invention further relates to a method for preparing the modified intermediates obtained after step b set forth above; basically this method is identical to the above method, however with the omission of step c.

The invention also relates to novel intermediates and donor substances used in the methods of the invention and the invention also relates to novel modified glycoproteins and novel intermediary modified glycoproteins obtainable by the methods of the present invention.

In the present text, some chemical structures are drawn with a “Me-” notation, a “CH3-” notation or just an ending solid line (chemical bond), which in all cases indicate a terminal methyl group. Some chemical structures may end in a dashed line, which means that the chemical structure is part of a larger structure.

### DETAILED DISCLOSURE OF THE INVENTION

The invention takes advantage of the “substrate tolerance” of many glycosyltransferases. Basically, any glycosyltransferase may be used (of course in a concentration that effectively catalyses the reaction between M′ and the donor substance). Examples of relevant enzymes can be found in the Nomenclature Committee of the International Union of Biochemistry and Molecular Biology (NC-IUBMB) class EC 2.4.1 (glycosyltransferases), EC 2.4.2 (pentosyltransferases) and EC 2.4.99 (covering enzymes transferring other glycosyl groups), which includes for illustration and not limitation: sialyltransferases, galactosyltransferases, N-acetylhexosaminyltransferases, glycosyltransferases, mannosyltransferases, fucosyltransferases, arabinosyltransferases, xylosyltransferase, glucuronosyltransferases, N-acetylglucosaminyltransferase and N-acetylgalactosaminyltransferases.

In some embodiments of the present invention, the starting molecule is a glycosylated polypeptide or protein. In the present specification and claims, the term “polypeptide” is a linear, single chain molecule consisting of peptide-bonded amino acid residues. Hence, the term embraces peptides (2-10 amino acid residues), oligopeptides (11-100 amino acid residues) and proper polypeptides (in excess of 100 amino acid residues). A polypeptide is thus a structural unit, which may be biologically active, but it can also lack any function. A “protein” is in the present context a functional or non-functional molecule or complex comprising at least one polypeptide, so apart from monomers, the term also includes polymeric molecules such as homo- and heteromultimers. A protein may include prosthetic groups, and may include various glycoslylation and lipidation patterns. In some embodiments of the invention, the polypeptide or protein is N-glycosylated or O-glycosylated.

In another embodiment, the method for producing the modified glycosylated molecule comprises the further step of confirming that the modified analogue has improved pharmacologic properties compared to the glycosylated starting molecule. Typically, the improved pharmacologic property is selected from the group consisting of increased bioavailability, increased functional in vivo half-life, increased in vivo plasma half-life, reduced immunogenicity, increased protease resistance, increased affinity for albumin, improved affinity for a receptor, increased storage stability.

The term “functional in vivo half-life” is used in its normal meaning, i.e. the time at which 50% of the biological activity of the modified analogue or a reference molecule is still present in the body/target organ, or the time it takes for the activity of the modified analogue or reference molecule to drop to 50% of its peak value. As an alternative to determining functional in vivo half-life, “in vivo plasma half-life” may be determined, i.e., the time at which 50% of the modified analogues or reference molecules circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life. The functionality to be retained is normally selected from procoagulant, proteolytic, co-factor binding, receptor binding activity, or other type of biological activity associated with the particular protein.

The term “increased” as used about the functional in vivo half-life or plasma half-life indicates that the relevant half-life of the modified analogue is statistically significantly increased relative to that of a reference molecule, such as an otherwise identical glycoprotein which has, however, not been subjected to the method of the invention. Thus, the half-life is determined under comparable conditions. For instance the relevant half-life may be increased by at least about 25%, such as by at least about 50%, e.g., by at least about 100%, 150%, 200%, 250%, or 500%. In some embodiments, the modified analogues of the present invention exhibit an increase in half-life of at least about 0.25 h, preferably at least about 0.5 h, more preferably at least about 1 h, and most preferably at least about 2 h, relative to the half-life of a reference preparation.

Measurement of in vivo biological half-life can be carried out in a number of ways as described in the literature. An example using modified FVIIa (coagulation factor VIIa) of an assay for the measurement of in vivo half-life of rFVIIa and variants thereof is described in FDA reference number 96-0597. Briefly, FVIIa clotting activity is measured in plasma drawn prior to and during a 24-hour period after administration of the modified analogue. The median apparent volume of distribution at steady state is measured and the median clearance determined.

“Bioavailability” refers to the proportion of an administered dose of a glycoconjugate that can be detected in plasma at predetermined times after administration. Typically, bioavailability is measured in test animals by administering a dose of between about 25-250 μg/kg of the preparation; obtaining plasma samples at predetermined times after administration; and determining the content of glycoprotein in the samples using a suitable bioassay, or immunoassay, or an equivalent assay. The data are typically displayed graphically as [glycoprotein] v. time and the bioavailability is expressed as the area under the curve (AUC). Relative bioavailability of a test preparation refers to the ratio between the AUC of the test preparation and that of the reference preparation.

In some embodiments, the preparations of the present invention exhibit a relative bioavailability of at least about 110%, preferably at least about 120%, more preferably at least about 130% and most preferably at least about 140% of the bioavailability of a reference preparation. The bioavailability may be measured in any mammalian species, preferably dogs, and the predetermined times used for calculating AUC may encompass different increments from 10 min-8 h. Bioavailability may, for example, be measured in a dog model as follows: The experiment is performed as a four leg cross-over study in 12 Beagle dogs divided in four groups. All animals receive a test preparation A and a reference preparation B at a dose of about 90 μg/kg in a suitable buffer such as glycylglycine buffer (pH 5.5) containing sodium chloride (2.92 mg/ml), calcium chloride dihydrate (1.47 mg/ml), mannitol (30 mg/ml) and polysorbate 80. Blood samples are drawn at 10, 30, and 60 minutes and 2, 3, 4, 6 and 8 hours following the initial administration. Plasma is obtained from the samples and polypeptide is quantified by ELISA.

The term “Immunogenicity” of a preparation refers to the ability of the preparation, when administered to a human, to elicit a deleterious immune response, whether humoral, cellular, or both. In any human sub-population, there may exist individuals who exhibit sensitivity to particular administered proteins. Immunogenicity may be measured by quantifying the presence of anti-glycoprotein antibodies and/or glycoprotein responsive T-cells in a sensitive individual, using conventional methods known in the art. In some embodiments, the modified analogues of the present invention exhibit a decrease in immunogenicity in a sensitive individual of at least about 10%, preferably at least about 25%, more preferably at least about 40% and most preferably at least about 50%, relative to the immunogenicity for that individual of a reference preparation.

Immunogenicity of a drug also relates to the fact that proteinaceous drugs may be immunogenic in non-sensitive subjects, meaning that repeated administrations of the drug leads to continuous boosting of an immune response against the drug. This is in most cases undesirable because the immune response will interfere with the activity of the drug, whereby it becomes necessary to administer increasing dosages of the drug over time in order to provide a therapeutic effect. In some embodiments, the modified analogues of the present invention exhibit a decrease in immunogenicity in non-sensitive subjects of at least about 10%, preferably at least about 25%, more preferably at least about 40% and most preferably at least about 50%, relative to the immunogenicity for that individual of a reference preparation.

The term “protease protected” as used herein referring to a polypeptide means a polypeptide which has been chemically modified in order to render said compound resistant to the plasma peptidases or proteases. Proteases in plasma are known to be involved in the degradation of several peptide hormones and also play a role in degradation of larger proteins.

Resistance of a polypeptide to degradation by for instance dipeptidyl aminopeptidase IV (DPPIV) is determined by the following degradation assay: Aliquots of the polypeptide (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the peptide degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Peptides and their degradation products may be monitored by their absorbance at 220 nm (peptide bonds) or 280 nm (aromatic amino acids), and are quantified by integration of their peak areas related to those of standards. The rate of hydrolysis of a peptide by dipeptidyl aminopeptidase IV is estimated at incubation times which result in less than 10% of the peptide being hydrolysed.

The most abundant protein component in circulating blood of mammalian species is serum albumin, which is normally present at a concentration of approximately 3 to 4.5 grams per 100 milliters of whole blood. Serum albumin is a blood protein of approximately 70,000 daltons which provides several important functions in the circulatory system. For instance, it functions as a transporter of a variety of organic molecules found in the blood, as the main transporter of various metabolites such as fatty acids and bilirubin through the blood, and, owing to its abundance, as an osmotic regulator of the circulating blood. Serum albumin has a half-life of more than one week, and one approach to increasing the plasma half-life of peptides has been to derivatize the peptides with a chemical entity that binds to serum albumin. The term “albumin binder” refers to such chemical entities that are known to bind to plasma proteins, such as albumin. Albumin binding property may be determined as described in J. Med. Chem., 43, 2000, 1986-1992, which is incorporated herein by reference. Albumin binding moieties may include fatty acid derivatives, organic sulfated polyaromates such as cibacron, as well as peptides comprising less than 40 amino acid residues such as moieties disclosed in J. Biol. Chem. 277, 38 (2002) 35035-35043, which is incorporated herein by reference.

The modified analogues, such as glycoconjugates, prepared according to the present invention exhibit improved functional properties relative to reference preparations. The improved functional properties may include, without limitation, a) physical properties such as, e.g., improved storage stability; b) improved pharmacokinetic properties such as, e.g., increased bioavailability and half-life; and c) reduced immunogenicity in humans.

A reference preparation refers to a preparation comprising a polypeptide that has an amino acid sequence identical to that contained in the modified analogue of the invention to which it is being compared (such as, e.g., non-conjugated forms of wild-type protein or a particular variant or chemically modified form) but which is not conjugated to a protractor molecule(s) as found in the preparation of the invention. For example, reference preparations typically comprise non-conjugated glycoprotein.

Storage stability of a glycoprotein may be assessed by measuring (a) the time required for 20% of the bioactivity of a preparation to decay when stored as a dry powder at 25° C. and/or (b) the time required for a doubling in the proportion of predetermined degradation products, such as, e.g., aggregates, in the preparation.

In some embodiments, the modified analogues of the invention exhibit an increase of at least about 30%, preferably at least about 60% and more preferably at least about 100%, in the time required for 20% of the bioactivity to decay relative to the time required for the same phenomenon in a reference preparation, when both preparations are stored as dry powders at 25° C.

Bioactivity measurements may be performed in accordance with the kind of bioactivity associated with the particular protein; in case of, e.g., coagulation factors, bioactivity may be measured using any of a clotting assay, proteolysis assay, TF-binding assay, or TF-independent thrombin generation assay.

In some embodiments, the preparations of the invention exhibit an increase of at least about 30%, preferably at least about 60%, and more preferably at least about 100%, in the time required for doubling of predetermined degradation products, such as, e.g., aggregates, relative to a reference preparation, when both preparations are stored as dry powders at 25° C. The content of aggregates may, for example, be determined by gel permeation HPLC, or another type of well-known chromatography methods. In the case of coagulation factors, aggregates may be determined by gel permeation HPLC on a Protein Pak 300 SW column (7.5×300 mm) (Waters, 80013) as follows. The column is equilibrated with Eluent A (0.2 M ammonium sulfate, 5% isopropanol, pH adjusted to 2.5 with phosphoric acid, and thereafter pH is adjusted to 7.0 with triethylamine), after which 25 μg of sample is applied to the column. Elution is with Eluent A at a flow rate of 0.5 ml/min for 30 min, and detection is achieved by measuring absorbance at 215 nm. The content of aggregates is calculated as the peak area of the coagulation factors aggregates/total area of coagulation factor peaks (monomer and aggregates).

### The Substituents P and P′

In the following, the substituent P will be discussed. The substituent P′ is identical to P with the exception that P′ includes a reactive functional group. When the reaction in the method of the invention has been finalised, this functional group is either absent (e.g. when the reactive group is a leaving group or a group which takes part of e.g. a reaction which liberates H₂O) or rendered substantially inactive as a consequence of the reaction.

In one embodiment of the present invention, P is different from a biotinyl group.

In one embodiment of the invention, increased half-life is obtained by P being a group that increases molecular weight so that renal clearance is reduced or abolished and/or by P being a group that masks binding partners for hepatic receptors. In an alternative embodiment, the reduced immunogenicity is obtained by P being a group which blocks antibody binding to immunogenic sites. In yet another embodiment, improved affinity for albumin is obtained by P being a group which has high affinity for albumin. And in yet another embodiment improved affinity for a receptor is obtained by P being a group which specifically binds a surface receptor on a target cell.

The substituent P can be any functionality improving group, e.g. a “protractor group”. As used herein this means a group which upon conjugation to a protein or peptide increases the circulation half-life of said protein or peptide, when compared to the un-modified protein or peptide. The specific principle behind the protractive effect may be caused by increased size, shielding of peptide sequences that can be recognized by peptidases or antibodies, or masking of glycanes in such way that they are not recognized by glycan specific receptores present in e.g. the liver or on macrophages, preventing or decreasing clearance. The protractive effect of the protractor group can e.g. also be caused by binding to blood components such as albumin, or by specific or unspecific adhesion to vascular tissue. The conjugated glycoprotein should substantially preserve biological activity of the non-modified glycoprotein.

Other possibilities include those where P is a group that targets the modified analogue to a certain type of cell or tissue, as is e.g. of interest if the glycoprotein has to exert its effect at a very high local concentration. Yet further possibilities include those where P is in its own right an active principle, e.g. a radionuclide or a toxic substance—this can e.g. be convenient in cases where the unmodified glycoprotein has high affinity for a receptor in malignant tissue and thus functions as a targeting moiety in the modified molecule.

In one embodiment of the invention P is selected from the group consisting of:
A low molecular organic charged radical (15-1000 Da), which may contain one or more carboxylic acids, amines sulfonic acids, phosphonic acids, or combination thereof,
A low molecular (15-1000 Da) neutral hydrophilic molecule, such as cyclodextrin, or a polyethylene chain which may optionally branched,
A low molecular (15-1000 Da) hydrophobic molecule such as a fatty acid or cholic acid or derivatives thereof,
Polyethyleneglycol with an average molecular weight of 2-40 KDa,
A well defined precision polymer such as a dendrimer with an exact molecular mass ranging from 700 to 20.000 Da, or more preferably between 700-10.000 Da,
A substantially non immunogenic polypeptide such as albumin or an antibody or part of an antibody optionally containing a Fc-domain, and
A high molecular weight organic polymer such as dextran.

In one embodiment of the invention the polymeric molecule is selected from the group consisting of dendrimers (e.g. with a molecular weight in the range of 700-10.000 Da or dendrimers as disclosed in International Patent Application WO 2005014049), polyalkylene oxide (PAO), including polyalkylene glycol (PAG), such as polyethylene glycol (PEG) and polypropylene glycol (PPG), branched PEGs, polyvinyl alcohol (PVA), polycarboxylate, polyvinylpyrolidone, polyethylene-co-maleic acid anhydride, polystyrene-co-maleic acid anhydride, and dextran, including carboxymethyl-dextran. In one embodiment of the invention, the polymeric molecule is a PEG group. In one embodiment of the invention, the polymeric molecule is a dendrimer.

In one embodiment of the invention, P is a protractor group selected from the group consisting of serum protein binding-ligands, such as serum protein binding-ligands, such as compounds which bind to albumin, such as fatty acids, C5-C24 fatty acid, aliphatic diacid (e.g. C5-C24), a structure (e.g. sialic acid derivatives or mimetics) which inhibits the glycans from binding to receptors (e.g. asialoglycoprotein receptor and mannose receptor), a small organic molecule containing moieties that under physiological conditions alters charge properties, such as carboxylic acids or amines, or neutral substituents that prevent glycan specific recognition such as smaller alkyl substituents (e.g., C1-C5 alkyl), a low molecular organic charged radical (e.g. C1-C25), which may contain one or more carboxylic acids, amines, sulfonic, phosphonic acids, or combination thereof; a low molecular neutral hydrophilic molecule (e.g. C1-C25), such as cyclodextrin, or a polyethylene chain which may optionally branched; polyethyleneglycol with an average molecular weight of 2-40 KDa; a well defined precision polymer such as a dendrimer with an exact molecular mass ranging from 700 to 20.000 Da, or more preferably between 700-10.000 Da; and a substantially non-immunogenic polypeptide such as albumin or an antibody or part of an antibody optionally containing a Fc-domain.

P may be an organic radical selected from one of the groups below:
straight, branched and/or cyclic C₁₋₃₀alkyl, C₂₋₃₀alkenyl, C₂₋₃₀alkynyl, C₁₋₃₀heteroalkyl, C₂₋₃₀heteroalkenyl, C₂₋₃₀heteroalkynyl, wherein one or more homocyclic aromatic compound biradical or heterocyclic compound biradical may be inserted, and wherein said C₁₋₃₀ or C₂₋₃₀ radicals may optionally be substituted with one or more substituents selected from —CO₂H, —SO₃H, —PO₂OH, —SO₂NH₂, —NH₂, —OH, —SH, halogen, or aryl, wherein said aryl is optionally substituted with —CO₂H, —SO₃H, —PO₂OH, —SO₂NH₂, —NH₂, —OH, —SH, or halogen; steroid radicals; lipid radicals;
polysaccharide radicals, e.g. dextrans; α-, β-; or γ-cyclodextrin, polyamide radicals e.g. polyamino acid radicals; PVP radicals; PVA radicals; poly(1-3-dioxalane); poly(1,3,6-trioxane); ethylene/maleic anhydride polymer;
Cibacron dye stuffs, such as Cibacron Blue 3GA, and polyamide chains of specified length, as disclosed in WO 00/12587, which is incorporated herein by reference;
a substantially non-immunogenic protein residue such as a blood component like albuminyl derivative, or an antibody or a domain thereof such as a Fc domain from human normal IgG1, as described in Kan, S K et al in The Journal of Immunology 2001, 166(2), 1320-1326 or in Stevenson, G T, The Journal of Immunology 1997, 158, 2242-2250;
polyethylene glycol (PEG) or methoxy polyethylene glycol (mPEG) radicals and amino derivatives thereof, where the average molecular weight may be between 500 and 100,000 Da, such as between 500 and 60,000 Da, such as between 1000 and 40,000 Da, such as between 5000 and 40,000 Da;
moieties that are known to bind to plasma proteins, such as e.g. albumin, where the albumin binding property may be determined as described in J. Med. Chem., 43, 2000, 1986-1992, which is incorporated herein by reference, or an albumin binding moiety such as a peptide comprising less than 40 amino acid residues such as moieties disclosed in J. Biol. Chem. 277, 38 (2002) 35035-35043, which is incorporated herein by reference.

In other embodiments, P is C₁-C₂₀-alkyl, such as C₁-C₁₈-alkyl. Specific mentioning is made of C₁₄-, C₁₆- and C₁₈-alkyl, which optionally may be substituted with in particular charged groups, polar groups and/or halogens. Examples of such substituents include —CO₂H and halogen. In a particular embodiment, all hydrogens in the C₁-C₂₀-alkyl are substituted with fluoro to form perfluoroalkyl.

To be able to react with the functional group comprised in B or L′, P′ comprises a functional group selected from the group consisting of any free amino, carboxyl, thiol, alkyl halide, acyl halide, chloroformiate, aryloxycarbonate, hydroxyl, α-haloacetamide, maleimide, azide, carbonyl group or aldehyde group; a carbonate such as p-nitrophenyl or succinimidyl; carbonyl imidazole; carbonyl chloride; carboxylic acid activated in situ; carbonyl halides; an activated ester such as an N-hydroxysuccinimide ester, an N-hydroxybenzotriazole ester, esters such as those comprising 1,2,3-benzotriazin-4(3H)-one; phosphoramidite; H-phosphonates; a phosphor triester or phosphor diester activated in situ; isocyanates; isothiocyanates; NH₂, OH, N₃, NHR′, OR′, O—NH₂, SH, alkynes,

| | |
|---|---|
| hydrazine derivatives | —NH—NH₂, |
| hydrazine carboxylate | —O—C(O)—NH—NH₂, |
| derivatives | |
| semicarbazide derivatives | —NH—C(O)—NH—NH₂, |
| thiosemicarbazide | —NH—C(S)—NH—NH₂, |
| derivatives | |
| carbonic acid dihydrazide | —NHC(O)—NH—NH—C(O)—NH—NH₂, |
| derivatives | |
| carbazide derivatives | —NH—NH—C(O)—NH—NH₂, |
| thiocarbazide derivatives | —NH—NH—C(S)—NH—NH₂, |
| aryl hydrazine | —NH—C(O)—C₆H₄—NH—NH₂, |
| derivatives | |
| hydrazide derivatives | —C(O)—NH—NH₂; and |
| oxylamine derivatives, | —C(O)—O—NH₂, —NH—C(O)—O—NH₂ |
| such as | and —NH—C(S)—O—NH₂. |

### General Structure of Donor Substances

The nucleoside mono- or diphosphates used in the present invention as donor substances are in general described according to formula I,

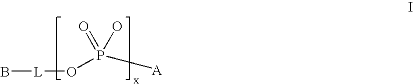

where x=1 or 2, and where A is one of either uridine, cytidine or guanosine connected via its 5′-hydroxyl group:

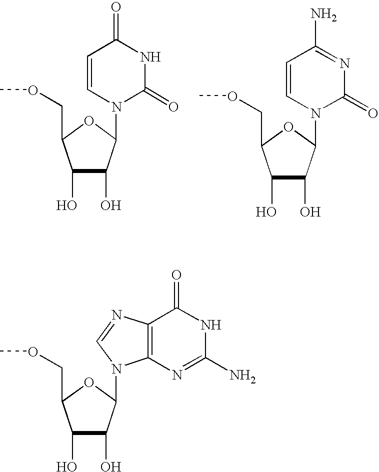

The Substituents B′ and B

In the following, the substituent B will be discussed. The substituent B is identical to B′ with the exception that B includes a reactive functional group. When the reaction in the methods of the invention has been finalised, this reactive functional group of B is either absent (e.g. when the reactive group is a leaving group or a group which takes part of e.g. a reaction which liberates H₂O) or rendered substantially inactive as a consequence of the reaction. B is only present when L is not identical to L′.

B conveniently comprises a reactive group that specifically can react with other suitable reactive groups such as nucleophiles, electrophiles, dienes, dienophiles, alkynes, and azides, preferably under mild conditions. Examples of B groups includes, by illustration and not limitation, α-haloacetamides, maleimides, azides, alkynes, and carbonyl groups such as ketones and aldehydes, thiohydryl groups, diene and dienophiles as disclosed in US 20040082067 A1, iodobenzoates or iodobenzamides as described in H. Dibowski and F. P. Schmidtchen, Angew. Chem. Int. Ed. 1998, 37 (4), 476-478, or functional groups as disclosed in Danish Patent Application PA 2003 01496 all suitable for ligation chemistry.

In one embodiment, B comprises a functional group selected from the group consisting of any free amino, carboxyl, thiol, alkyl halide, acyl halide, chloroformiate, aryloxycarbonate, hydroxyl, α-haloacetamide, maleimide, azide, carbonyl group or aldehyde group; a carbonate such as p-nitrophenyl or succinimidyl; carbonyl imidazole; carbonyl chloride; carboxylic acid activated in situ; carbonyl halides; an activated ester such as an N-hydroxysuccinimide ester, an N-hydroxybenzotriazole ester, esters such as those comprising 1,2,3-benzotriazin-4(3H)-one; phosphoramidite; H-phosphonates; a phosphor triester or phosphor diester activated in situ; isocyanates; isothiocyanates; NH₂, OH, N₃, NHR′, OR′, O—NH₂, SH, alkynes,

hydrazine derivatives —NH—NH₂,
hydrazine carboxylate derivatives —O—C(O)—NH—NH₂,
semicarbazide derivatives —NH—C(O)—NH—NH₂,
thiosemicarbazide derivatives —NH—C(S)—NH—NH₂,
carbonic acid dihydrazide derivatives —NHC(O)—NH—NH—C(O)—NH—NH₂,
carbazide derivatives —NH—NH—C(O)—NH—NH₂,
thiocarbazide derivatives —NH—NH—C(S)—NH—NH₂,
aryl hydrazine derivatives —NH—C(O)—C₆H₄—NH—NH₂,
hydrazide derivatives —C(O)—NH—NH₂; and
oxylamine derivatives, such as —C(O)—O—NH₂, —NH—C(O)—O—NH₂ and —NH—C(S)—O—NH₂.

The functional group comprised in P′ and B or L′ may in principle be selected from the same list of groups. It is, however, to be understood that this selection is made so that the two functional groups are capable of reacting with each other.

### The Substituents L and L′

In the following, the substituent L will be discussed. The substituent L′ is identical to L with the exception that L′ includes a reactive functional group. When the reaction in the method of the invention has been finalised, this functional group is either absent (e.g. when the reactive group is a leaving group or a group which takes part of e.g. a reaction which liberates H₂O) or rendered substantially inactive as a consequence of the reaction.

L is a linker moiety, preferably in the form of a divalent organic radical. L can be linear, in which case it preferably includes a multiply functionalized alkyl group containing up to 18, and more preferably between 2-10, carbon atoms. Several heteroatoms, such as nitrogen, oxygen or sulphur, may be included within the alkyl chain. The alkyl chain may also be branched at a carbon or a nitrogen atom. In special cases, L is a simple valence bond.

Alternatively L can be a 5-7 membered ring, optionally containing one or more heteroatoms, selected independently from nitrogen, oxygen or sulfur.

In an embodiment, L provides for an oxygen, nitrogen or sulfur containing heterocycle of 5 to 7 ring atoms to result in general formula Ia-Ic:

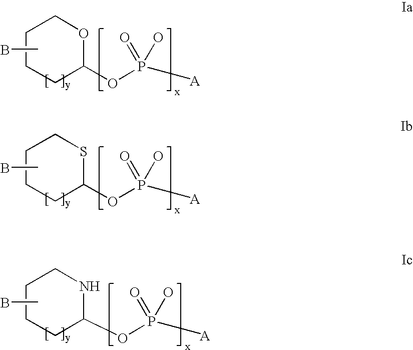

where y=0, 1, 2, and A and B are as defined supra. Each ring carbon may optionally be substituted with hydroxyl groups, with hydroxymethyl groups, N-acylamino groups, alkyl, alkyloxy, halogene, alkanoyl, aryl, aryloxy, heteroaryl and heteroaryloxy groups, with all possible stereo isomeric forms included.

In an other embodiment, L provides for an acyl group, resulting in the donor substance having general formula Id:

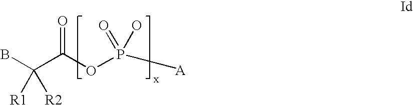

with A, B and x defined supra and R1 and R2 each independently selected from alkyl, halogen, alkanoyl, aryl and heteroaryl.

In yet an other embodiment, L is derived from a carbohydrate moiety of general formula as shown below:

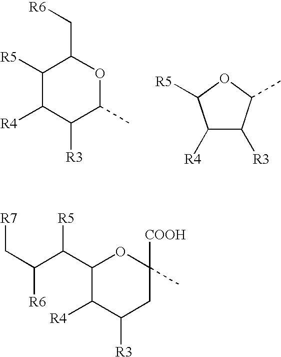

where one of the substituents R3-R7 is being selected from any divalent organic radical (attached to B) and the remaining R3-R7 are selected independently from —H, —OH, —CH₂OH, —NH₂, N-acylamino groups including —NHAc, alkyl, alkyloxy, halogene, alkanoyl, aryl, aryloxy, heteroaryl or heteroaryloxy groups, with all possible stereo isomeric forms included. R3-R7 may alternatively be a valence bond directly connected to B.

In one embodiment, B in general formula I is absent, and L (i.e. L′) is derived from an oxidized carbohydrate moiety of general formula as shown below:

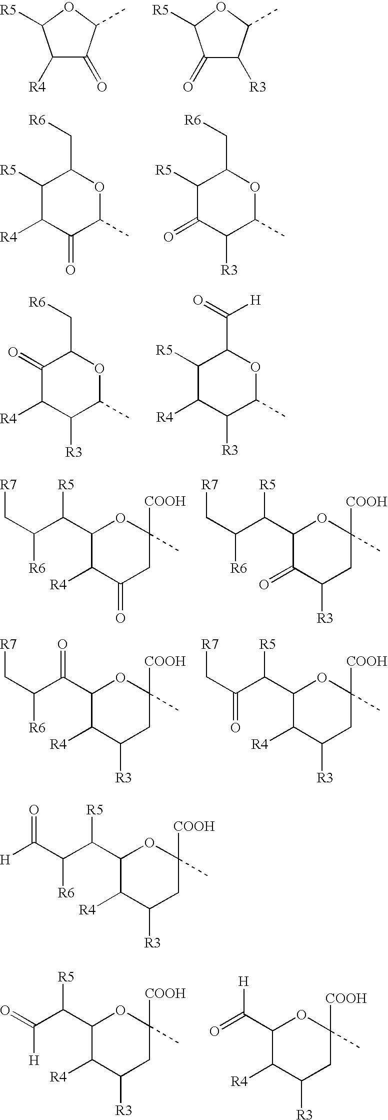

where R3-R7 independently are selected from —H, —OH, —NH₂, N-acylamino groups including —NHAc, —CH₂OH, alkyl, alkyloxy, halogene, alkanoyl, aryl, aryloxy, heteroaryl or heteroaryloxy groups, with all possible stereo isomeric forms, or germinal diol forms included.

### Embodiments of the Donor Substance

In an embodiment, formula I is:

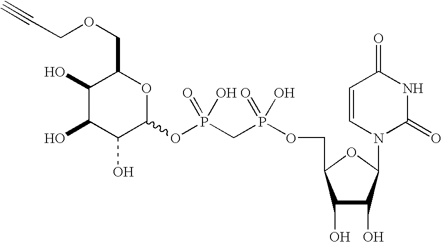

or any stereo isomers or salts thereof such as mono-, di-, tri, or tetraalkylammonium, sodium, potassium etc.

In an embodiment, formula I is:

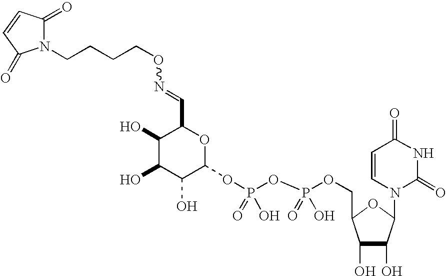

or any stereo isomers or other salts thereof such as mono-, di-, tri, or tetraalkylammonium, potassium, ammonium etc.

In an embodiment, formula I is:

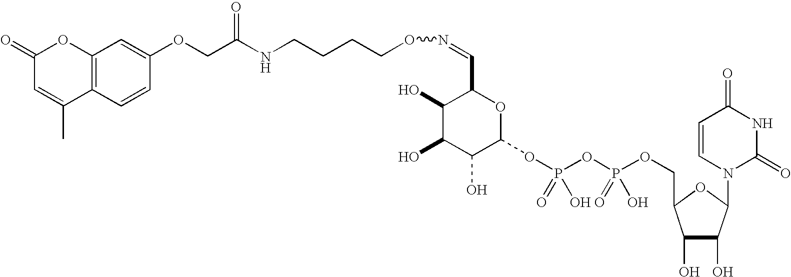

or any stereo isomers or salts thereof such as mono-, di-, tri, or tetraalkylammonium, sodium, potassium etc.

In an embodiment formula I is:

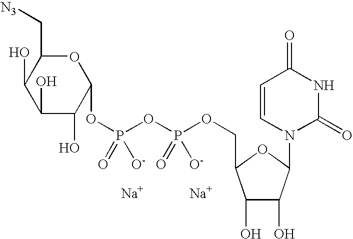

or any stereo isomers or other salts thereof such as mono-, di-, tri, or tetraalkylammonium, potassium, ammonium etc.

In an embodiment, formula I is:

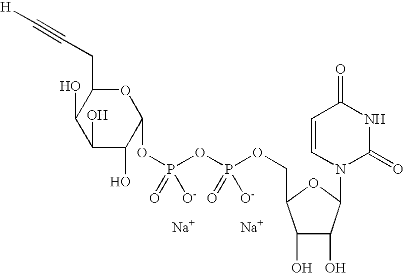

or any stereo isomers or other salts thereof such as mono-, di-, tri, or tetraalkylammonium, potassium, ammonium etc.

In an embodiment, formula I is:

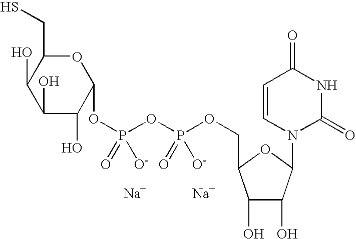

or any stereo isomers or other salts thereof such as mono-, di-, tri, or tetraalkylammonium, potassium, ammonium etc. The thiol group can optionally be protected as a mixed disulfide.

In an embodiment, formula I together is one of either:

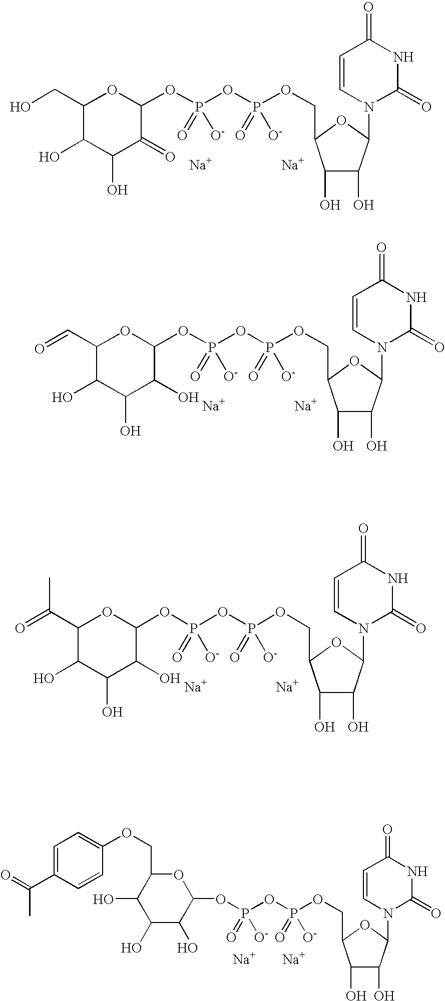

including any stereo isomers or germinal diol forms or other salts thereof such as mono-, di-, tri, or tetraalkylammonium, ammonium, potassium etc.

In an embodiment, formula I is:

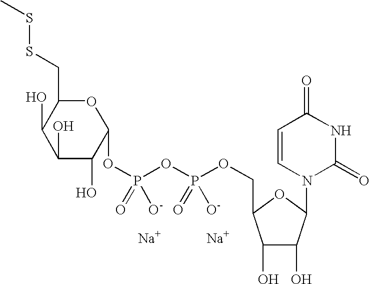

or any stereo isomers or salts thereof such as mono-, di-, tri, or tetraalkylammonium, sodium, potassium etc.

In an embodiment, formula I is:

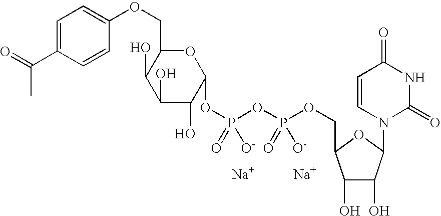

or any stereo isomers or salts thereof such as mono-, di-, tri, or tetraalkylammonium, sodium, potassium etc.

In an embodiment, formula I is:

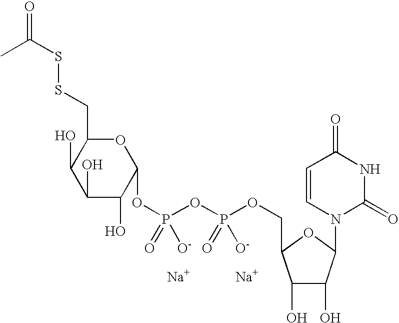

or any stereo isomers or salts thereof such as mono-, di-, tri, or tetraalkylammonium, sodium, potassium etc.

In an embodiment, formula I is:

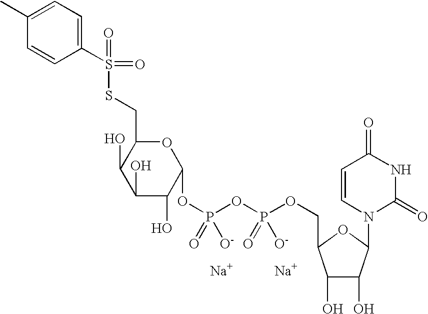

or any stereo isomers or salts thereof such as mono-, di-, tri, or tetraalkylammonium, sodium, potassium etc.

In an embodiment, formula I is:

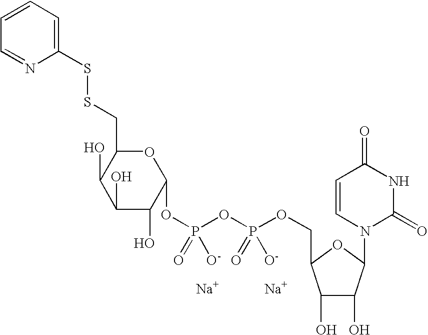

or any stereo isomers or salts thereof such as mono-, di-, tri, or tetraalkylammonium, sodium, potassium etc.

In an embodiment, formula I is:

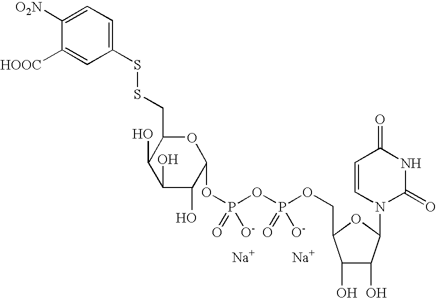

or any stereo isomers or salts thereof such as mono-, di-, tri, or tetraalkylammonium, sodium, potassium etc.

In an embodiment, formula I is:

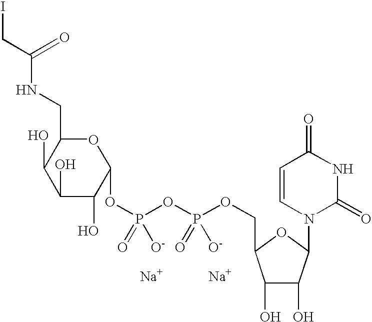

or any stereo isomers or salts thereof such as mono-, di-, tri, or tetraalkylammonium, sodium, potassium etc.

In an embodiment, formula I is:

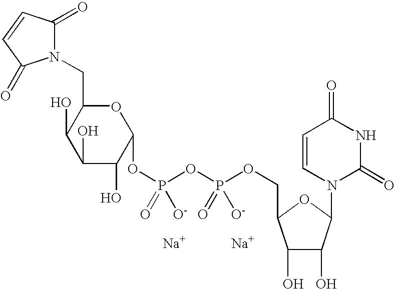

or any stereo isomers or salts thereof such as mono-, di-, tri, or tetraalkylammonium, sodium, potassium etc.

In an embodiment, formula I is:

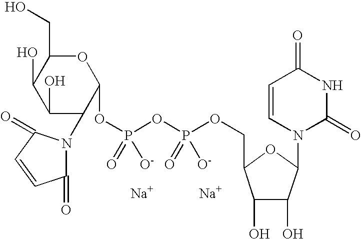

or any stereo isomers or salts thereof such as mono-, di-, tri, or tetraalkylammonium, sodium, potassium etc.

In an embodiment, formula I is:

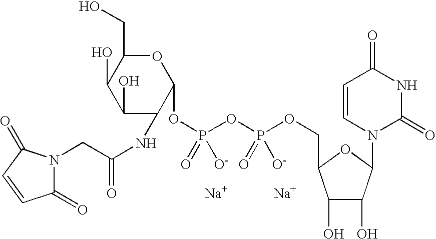

or any stereo isomers or salts thereof such as mono-, di-, tri, or tetraalkylammonium, sodium, potassium etc.

In an embodiment, formula I is:

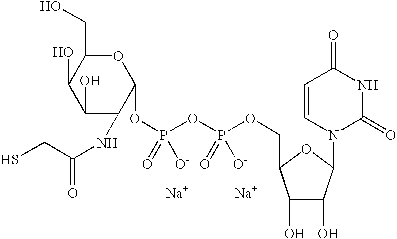

or any stereo isomers or salts thereof such as mono-, di-, tri, or tetraalkylammonium, sodium, potassium etc.

In an embodiment, formula I is:

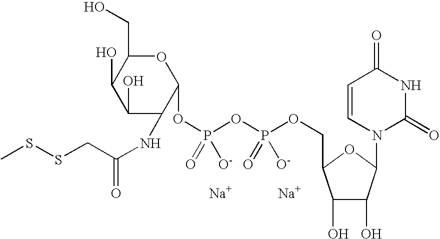

or any stereo isomers or salts thereof such as mono-, di-, tri, or tetraalkylammonium, sodium, potassium etc.

In an embodiment, formula I is:

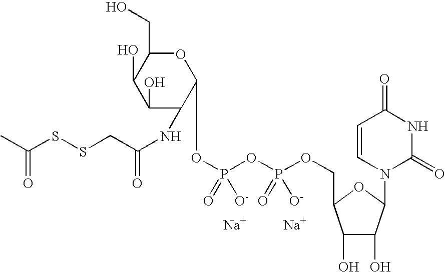

or any stereo isomers or salts thereof such as mono-, di-, tri, or tetraalkylammonium, sodium, potassium etc.

In an embodiment, formula I is:

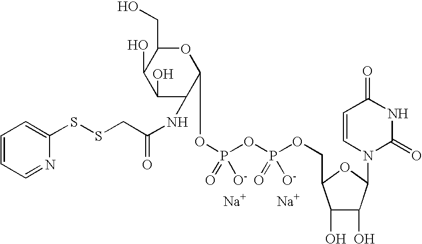

or any stereo isomers or salts thereof such as mono-, di-, tri, or tetraalkylammonium, sodium, potassium etc.

In an embodiment, formula I is:

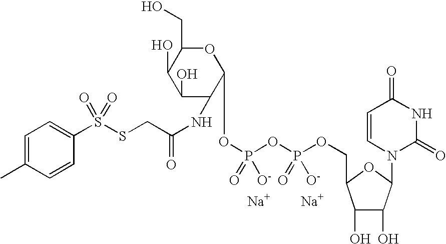

or any stereo isomers or salts thereof such as mono-, di-, tri, or tetraalkylammonium, sodium, potassium etc.

In an embodiment, formula I is:

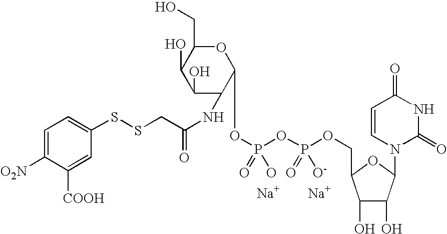

or any stereo isomers or salts thereof such as mono-, di-, tri, or tetraalkylammonium, sodium, potassium etc.

In an embodiment, formula I is:

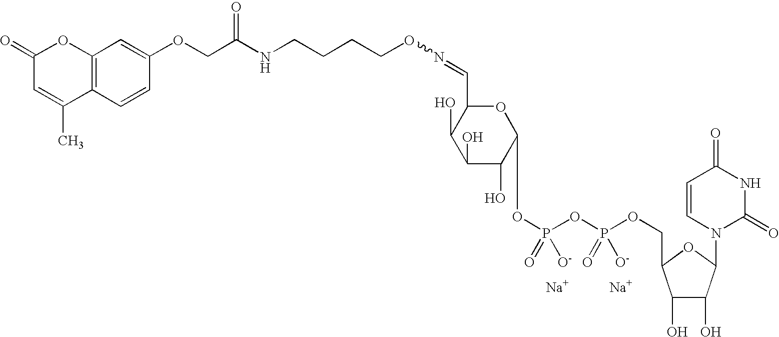

or any stereo isomers or salts thereof such as mono-, di-, tri, or tetraalkylammonium, sodium, potassium etc.

In an embodiment, formula I is:

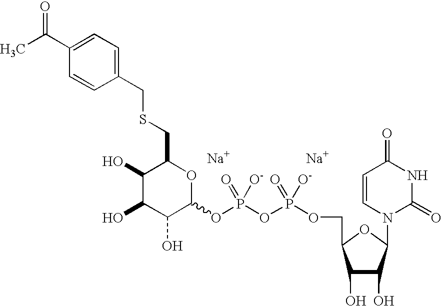

or any stereo isomers or salts thereof such as mono-, di-, tri, or tetraalkylammonium, sodium, potassium etc.

In an embodiment, formula I is:

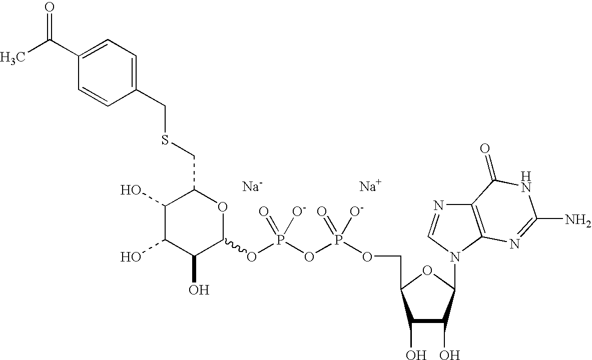

or any stereo isomers or salts thereof such as mono-, di-, tri, or tetraalkylammonium, sodium, potassium etc.

In an embodiment, formula I is:

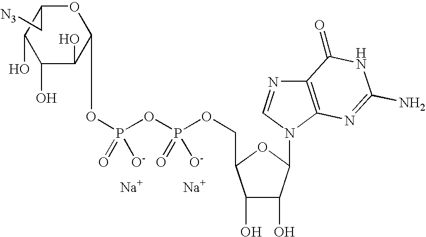

or any stereo isomers or salts thereof such as mono-, di-, tri, or tetraalkylammonium, sodium, potassium etc.

In an embodiment, formula I is:

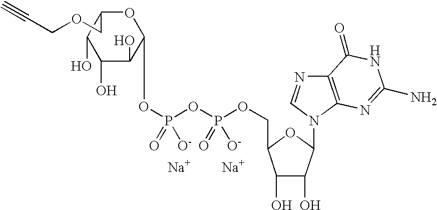

or any stereo isomers or salts thereof such as mono-, di-, tri, or tetraalkylammonium, sodium, potassium etc.

In an embodiment, formula I is:

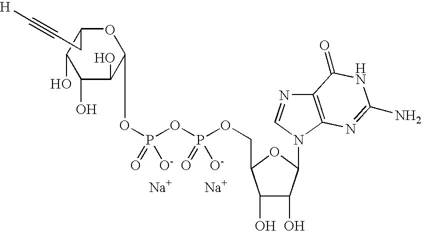

or any stereo isomers or salts thereof such as mono-, di-, tri, or tetraalkylammonium, sodium, potassium etc.

In an embodiment, formula I is:

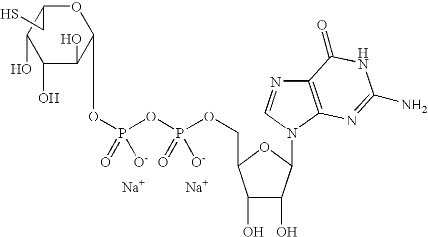

or any stereo isomers or salts thereof such as mono-, di-, tri, or tetraalkylammonium, sodium, potassium etc, including a compound where the thiol group is protected as a mixed disulfide.

In an embodiment, formula I is:

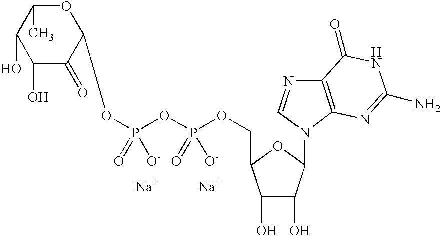

including any stereo isomers or germinal diol forms or other salts thereof such as mono-, di-, tri, or tetraalkylammonium, ammonium, potassium etc.

In an embodiment, formula I is:

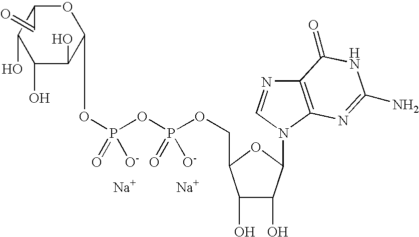

including any stereo isomers or germinal diol forms or other salts thereof such as mono-, di-, tri, or tetraalkylammonium, ammonium, potassium etc.

In an embodiment, formula I is:

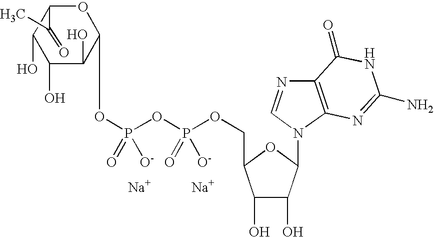

including any stereo isomers or germinal diol forms or other salts thereof such as mono-, di-, tri, or tetraalkylammonium, ammonium, potassium etc.

In an embodiment, formula I is:

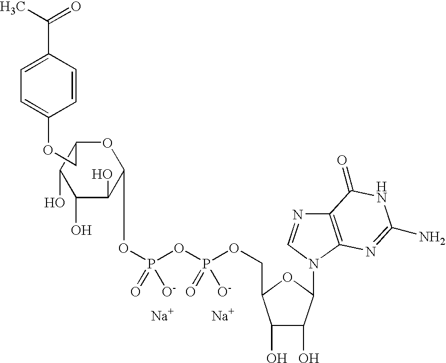

including any stereo isomers or germinal diol forms or other salts thereof such as mono-, di-, tri, or tetraalkylammonium, ammonium, potassium etc.

In an embodiment, formula I is:

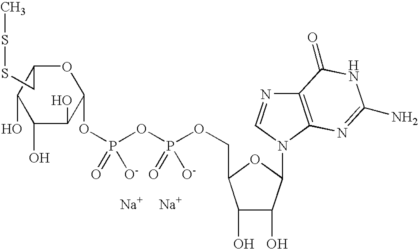

or any stereo isomers or salts thereof such as mono-, di-, tri, or tetraalkylammonium, sodium, potassium etc.

In an embodiment, formula I is:

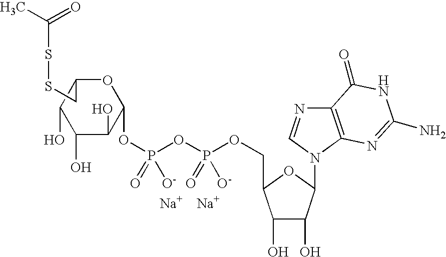

or any stereo isomers or salts thereof such as mono-, di-, tri, or tetraalkylammonium, sodium, potassium etc.

In an embodiment, formula I is:

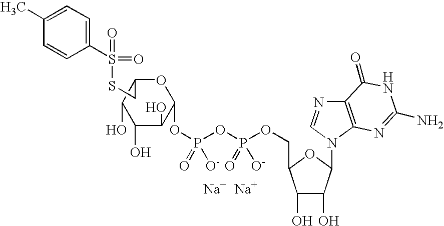

or any stereo isomers or salts thereof such as mono-, di-, tri, or tetraalkylammonium, sodium, potassium etc.

In an embodiment, formula I is:

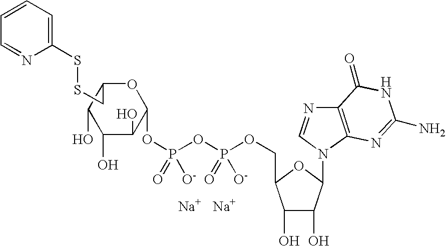

or any stereo isomers or salts thereof such as mono-, di-, tri, or tetraalkylammonium, sodium, potassium etc.

In an embodiment, formula I is:

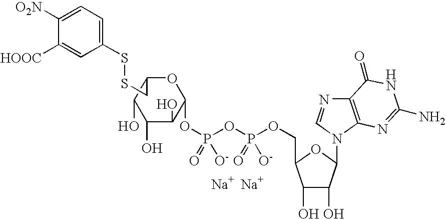

or any stereo isomers or salts thereof such as mono-, di-, tri, or tetraalkylammonium, sodium, potassium etc.

In an embodiment, formula I is:

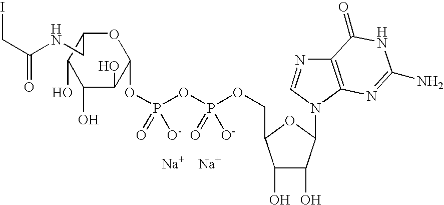

or any stereo isomers or salts thereof such as mono-, di-, tri, or tetraalkylammonium, sodium, potassium etc.

In an embodiment, formula I is:

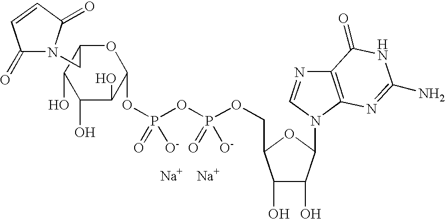

or any stereo isomers or salts thereof such as mono-, di-, tri, or tetraalkylammonium, sodium, potassium etc.

In an embodiment, formula I is:

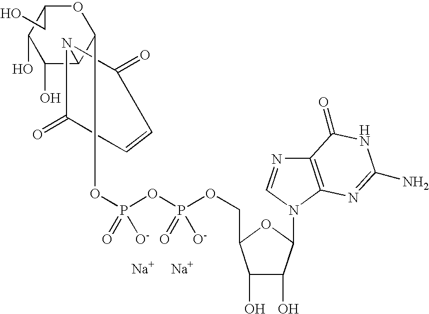

or any stereo isomers or salts thereof such as mono-, di-, tri, or tetraalkylammonium, sodium, potassium etc.

In an embodiment, formula I is:

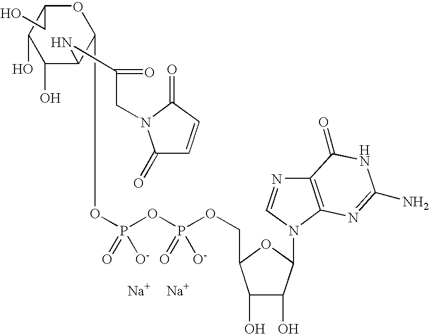

or any stereo isomers or salts thereof such as mono-, di-, tri, or tetraalkylammonium, sodium, potassium etc.

In an embodiment, formula I is:

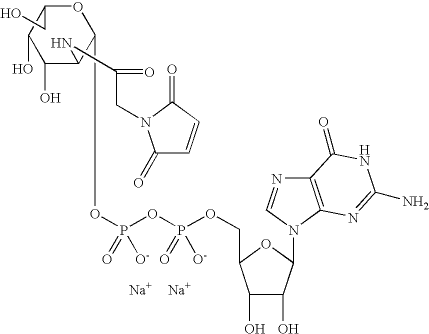

or any stereo isomers or salts thereof such as mono-, di-, tri, or tetraalkylammonium, sodium, potassium etc.

In an embodiment, formula I is:

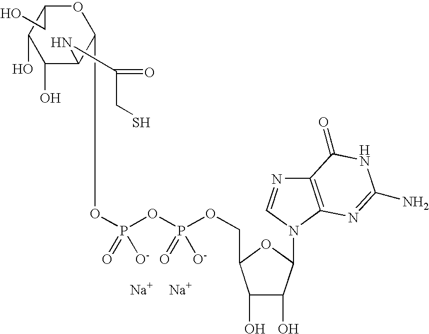

or any stereo isomers or salts thereof such as mono-, di-, tri, or tetraalkylammonium, sodium, potassium etc.

In an embodiment, formula I is:

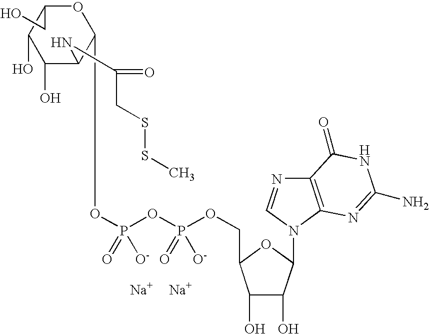

or any stereo isomers or salts thereof such as mono-, di-, tri, or tetraalkylammonium, sodium, potassium etc.

In an embodiment, formula I is:

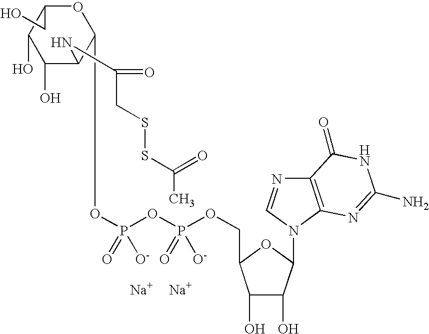

or any stereo isomers or salts thereof such as mono-, di-, tri, or tetraalkylammonium, sodium, potassium etc.

In an embodiment, formula I is:

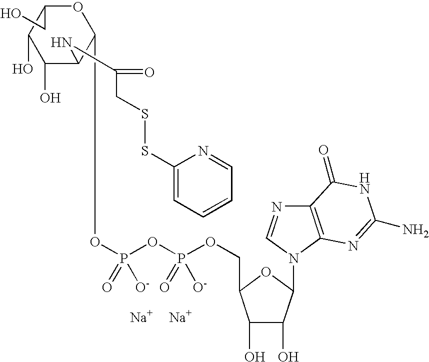

or any stereo isomers or salts thereof such as mono-, di-, tri, or tetraalkylammonium, sodium, potassium etc.

In an embodiment, formula I is:

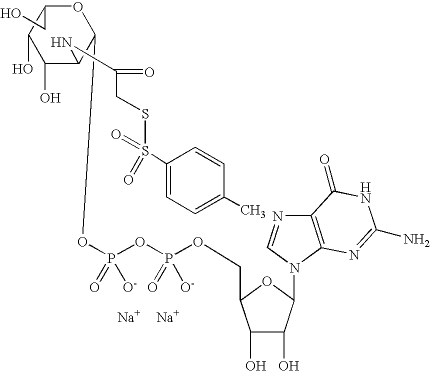

or any stereo isomers or salts thereof such as mono-, di-, tri, or tetraalkylammonium, sodium, potassium etc.

In an embodiment, formula I is:

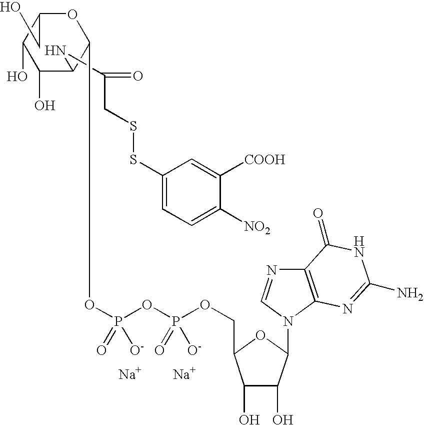

or any stereo isomers or salts thereof such as mono-, di-, tri, or tetraalkylammonium, sodium, potassium etc.

In an embodiment, formula I is:

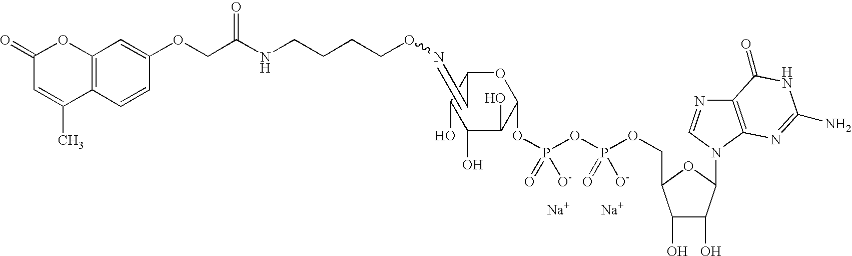

or any stereo isomers or salts thereof such as mono-, di-, tri, or tetraalkylammonium, sodium, potassium etc.

### The Starting Molecule M′

In the following, the substituent M′ will be discussed.

The substituent M′ comprises a polypeptide moiety and a reactive group, that function as an acceptor substrate for a glycosyltransferase.

When the reaction in the methods of the invention has been finalised, this functional group of M′ is either absent or rendered substantially inactive as a consequence of the reaction.

The reactive group in M′ acts as a glycosyltransferase acceptor substance, that together with a donor substance of general formula I and an appropriate glycosyltransferase, can form intermediate modified analogues of the formula B-L-M or L′-M.

The reactive group in M′ can a part of a carbohydrate, or derived from a carbohydrate residue such as those found in N- or O-glycanes of glycosylated polypeptides.

Alternatively the reactive group can be the side chain of a serine or threonine residue present in the polypeptide sequence, or the side chains of any of the following residues: lysine, asparagine, glutamine, tryptophane, tyrosine, cystine, arginine, histidine, glutamic acid, aspartic acid, hydroxyproline, gamma-carboxyglutamic acid.

Posttranslationally oxidized peptide residues such as hydroxyproline or hydroxylysine are also regarded as reactive groups according to the invention.

The C- and N-terminal of the polypeptide moiety of M′ may also act as reactive groups (e.g. the free carboxyl group, the free carboxamide, or the free amino group in the polypeptide termini).

In one embodiment M′ is selected from FVII, FVIII, FIX, FX, FII, FV, protein C, protein S, tPA, PAI-1, tissue factor, FXI, FXII, FXIII, as well as sequence variants thereof; immunoglobulins, cytokines such as interleukins, alpha-, beta-, and gamma-interferons, colony stimulating factors including granulocyte colony stimulating factors, platelet derived growth factors and phospholipase-activating protein (PUP). M′ can also be any other protein and peptide of general biological and therapeutic interest include insulin, plant proteins such as lectins and ricins, tumor necrosis factors and related alleles, soluble forms of tumor necrosis factor receptors, interleukin receptors and soluble forms of interleukin receptors, growth factors such as tissue growth factors, such as TGFa's or TGFps and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and immunoglobulins such as IgG, IgE, IgM, IgA, and IgD, and fragments thereof.

Peptides and proteins, that do not contain glycan moieties can be glycosylated either enzymatically as described in L1 Shao et all. Glycobiology 12(11) 762-770 (2002) using glycosyltransferases, or chemically synthesised, for example by using standard peptide chemistry and glycosylated amino acid components such as N-galactosylated asparagine.

Alternatively glycosylation sites may be engineered into proteins or peptides which in vivo normally are produced in their non-glycosylated form. For example insertion of the consensus sequence Cys-XXX-Ser-XXX-Pro-Cys in an EGF repeat allows for selective O-glycosylation of serine using UDP-Glucose and glucosyltransferase L1 Shao et all. Glycobiology 12(11) 762-770 (2002), whereas insertion of the consensus sequence Asn-XXX-Ser/Thr allows for N-glycosylation R. A. Dwek, Chem. Rev. 1996, 96, 683-720. Peptide sequences containing threonine or serine also undergoes glycosylation in the presence of UDP-GalNAc:polypeptide N-acetylgalactosaminyltransferase and UDP-GalNAc in a sequence dependent manner (see for example B. C. O'Connell, F. K. Hagen and L. A. Tabak in 3. Biol. Chem. 267(35), 25010-25018 (1992)). Alternatively site directed mutagenesis introducing cysteine mutations can be used for introduction of galactose or galactose containing sugar structures via mixed disulphide formation as described by D. P. Gamblin et al. in Angew. Chem. Int. Ed., 43, 828 (2004). Galactose or N-acetylgalactosamine containing peptide and proteins can also be made by conjugation to proteins or peptides containing non-biogenical handles such as methods described by P. G. Schultz in J. Am. Chem. Soc, 125, 1702 (2003), or unspecifically by direct glycosylation of peptides using glycosyl donor substrates such as trichloroacetamidyl galactosides etc. Addition of glycosidase inhibitors to fermentation cultures, thereby producing glycoproteins with truncated glycan structures as described in U.S. Pat. No. 4,925,796A/U.S. Pat. No. 5,272,066A1 is also a possibility for obtaining galactose or N-acetylgalactosamine containing proteins, as well as enzymatic modification of glutamine residues using TGase (see for example M. Sato et al. Angew. Chem. Int. Ed. 43, 1516-1520, (2004).

Production of N-glycosylated proteins are not limited to the use of mammalian host cells such as CHO or BHK cells, but also can be performed in insect cells, yeast, or by using bacterial cells as described by M. Wacker et al. in Science, 298, 1790-1793 (2002).

In an embodiment of the invention the peptide is aprotinin, tissue factor pathway inhibitor or other protease inhibitors, insulin or insulin precursors, human or bovine growth hormone, interleukin, glucagon, oxyntomodulin, GLP-1, GLP-2, IGF-I, IGF-II, tissue plasminogen activator, transforming growth factor γ or β, platelet-derived growth factor, GRF (growth hormone releasing factor), human growth factor, immunoglobulines, EPO, TPA, protein C, blood coagulation factors such as FVII, FVIII, FIX, FX, FII, FV, protein C, protein S, PAI-1, tissue factor, FXI, FXII, and FXIII, exendin-3, exentidin-4, and enzymes or functional analogues thereof. In the present context, the term “functional analogue” is meant to indicate a protein with a similar function as the native protein. The protein may be structurally similar to the native protein and may be derived from the native protein by addition of one or more amino acids to either or both the C and N-terminal end of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Furthermore the protein may be acylated in one or more positions, see, e.g., WO 98/08871, which discloses acylation of GLP-1 and analogues thereof, and WO 98/08872, which discloses acylation of GLP-2 and analogues thereof. An example of an acylated GLP-1 derivative is Lys26(N^{epsilon}-tetradecanoyl)-GLP-1 (7-37) which is GLP-1 (7-37) wherein the epsilon-amino group of the Lys residue in position 26 has been tetradecanoylated.

The proteins or portions thereof can be prepared or isolated by using techniques known to those of ordinary skill in the art such as tissue culture, extraction from animal sources, or by recombinant DNA methodologies. Transgenic sources of the proteins, peptides, amino acid sequences and the like are also contemplated. Such materials are obtained form transgenic animals. i.e., mice, pigs, cows, etc., wherein the proteins expressed in milk, blood or tissues. Transgenic insects and baculovirus expression systems are also contemplated as sources. Moreover, mutant versions, of proteins, such as mutant TNF's and/or mutant interferons are also within the scope of the invention. Other proteins of interest are allergen proteins such as ragweed, Antigen E, honeybee venom, mite allergen, and the like.

The foregoing is illustrative of the biologically active peptides which are suitable for conjugation with a protractor group in accordance with the invention. It is to be understood that those biologically active materials not specifically mentioned but having suitable peptides are also intended and are within the scope of the present invention.

In one embodiment, the glycoprotein is a FVII polypeptide. In one embodiment, the polypeptides are wild-type Factor VIIa.

As used herein, the terms “Factor VII polypeptide” or “FVII polypeptide” means any protein comprising the amino acid sequence 1-406 of wild-type human Factor VIIa (i.e., a polypeptide having the amino acid sequence disclosed in U.S. Pat. No. 4,784,950), as well as variants thereof.

The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. Such variants of Factor VII may exhibit different properties relative to human Factor VII, including stability, phospholipid binding, altered specific activity, and the like.

As used herein, “wild type human FVIIa” is a polypeptide having the amino acid sequence disclosed in U.S. Pat. No. 4,784,950.

Non-limiting examples of Factor VII variants include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified Gla-domain and exhibiting an enhanced membrane binding as disclosed in WO 99/20767, U.S. Pat. No. 6,017,882 and U.S. Pat. No. 6,747,003, US patent application 20030100506 (University of Minnesota) and WO 00/66753, US patent applications US 20010018414, US 2004220106, and US 200131005, U.S. Pat. No. 6,762,286 and U.S. Pat. No. 6,693,075 (University of Minnesota); and FVII variants as disclosed in WO 01/58935, U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

Non-limiting examples of FVII variants having increased biological activity compared to wild-type FVIIa include FVII variants as disclosed in WO 01/83725, WO 02/22776, WO 02/077218, PCT/DK02/00635 (corresponding to WO 03/027147), Danish patent application PA 2002 01423 (corresponding to WO 04/029090), Danish patent application PA 2001 01627 (corresponding to WO 03/027147); WO 02/38162 (Scripps Research Institute); and FVIIa variants with enhanced activity as disclosed in JP 2001061479 (Chemo-Sero-Therapeutic Res Inst.).

Examples of variants of factor VII include, without limitation, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn; FVII having substitutions, additions or deletions in the amino acid sequence from 304Arg to 329Cys; and FVII having substitutions, additions or deletions in the amino acid sequence from 153Ile to 223Arg.

### Other Aspects of the Invention

The method steps a and b in the method of the invention are believed to be novel and inventive in their own right. These two steps provide for the intermediary modified glycosylated analogue which is a convenient “ready-to-conjugate” molecule, where various groups P can be attached. For screening and testing purposes, this provides for a simple means of preparing a panel or even library of modified analogues—all which is required is that the various P′ groups include a reactive group that can react with the reactive group in B or L′.

Hence, the invention also pertains to a method for the preparation of a modified intermediate of formula B-L-M or L′-M, said method omitting step c of the above-detailed method. Furthermore, the invention also relates to such novel intermediates as such which have the formula B-L-M or L′-M.

Such a modified intermediate is in some embodiments of the invention selected from modified FVII, FVIII, FIX, FX, FII, FV, protein C, protein S, tPA, PAI-1, tissue factor, FXI, FXII, FXIII, as well as sequence variants thereof; immunoglobulins, cytokines such as interleukins, alpha-, beta-, and gamma-interferons, colony stimulating factors including granulocyte colony stimulating factors, platelet derived growth factors and phospholipase-activating protein (PUP).

Other modified intermediates of formula B-L-M or L′-M are modified proteins and peptides of general biological and therapeutic interest, e.g. including insulin, plant proteins such as lectins and ricins, tumor necrosis factors and related alleles, soluble forms of tumor necrosis factor receptors, interleukin receptors and soluble forms of interleukin receptors, growth factors such as tissue growth factors, such as TGFa's or TGFps and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and immunoglobulins such as IgG, IgE, IgM, IgA, and IgD, and fragments thereof.

Finally, the present invention also pertains to novel modified analogues of formula P—B′-L-M or P-L-M.

Such a modified analogue is in some embodiments of the invention selected from modified FVII, FVIII, FIX, FX, FII, FV, protein C, protein S, tPA, PAI-, tissue factor, FXI, FXII, FXIII, as well as sequence variants thereof; immunoglobulins, cytokines such as interleukins, alpha-beta-, and gamma-interferons, colony stimulating factors including granulocyte colony stimulating factors, platelet derived growth factors and phospholipase-activating protein (PUP).

Other modified analogues of formula P—B′-L-M or P-L-M are modified proteins and peptides of general biological and therapeutic interest, e.g. including insulin, plant proteins such as lectins and ricins, tumor necrosis factors and related alleles, soluble forms of tumor necrosis factor receptors, interleukin receptors and soluble forms of interleukin receptors, growth factors such as tissue growth factors, such as TGFa's or TGFps and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and immunoglobulins such as IgG, IgE, IgM, IgA, and IgD, and fragments thereof.

In one embodiment, the method for production of the modified glycosylated molecules comprises the further step of formulating said glycosylated molecule as a pharmaceutical composition.

### Pharmaceutical Compositions

Another object of the present invention is to provide a pharmaceutical composition comprising a modified analogue which is present in a concentration from 10⁻¹² mg/ml to 200 mg/ml, such as e.g. 10⁻¹⁰ mg/ml to 5 mg/ml and wherein said composition has a pH from 2.0 to 10.0. The composition may further comprise a buffer system, preservative(s), tonicity agent(s), chelating agent(s), stabilizers and surfactants. In one embodiment of the invention the pharmaceutical composition is an aqueous composition, i.e. composition comprising water. Such composition is typically a solution or a suspension. In a further embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical composition is a freeze-dried composition, whereto the physician or the patient adds solvents and/or diluents prior to use.

In another embodiment the pharmaceutical composition is a dried composition (e.g. freeze-dried or spray-dried) ready for use without any prior dissolution.

In a further aspect the invention relates to a pharmaceutical composition comprising an aqueous solution of a Modified analogue, and a buffer, wherein said Modified analogue is present in a concentration from 0.1-100 mg/ml or above, and wherein said composition has a pH from about 2.0 to about 10.0.

In another embodiment of the invention the pH of the composition is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the composition further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In a further embodiment of the invention the composition further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In a further embodiment of the invention the composition further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In a further embodiment of the invention the composition further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a protein that possibly exhibits aggregate formation during storage in liquid pharmaceutical compositions. By “aggregate formation” is intended a physical interaction between the protein molecules that results in formation of oligomers, which may remain soluble, or large visible aggregates that precipitate from the solution. By “during storage” is intended a liquid pharmaceutical composition or composition once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or composition is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions of the invention may further comprise an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L or D isomer, or mixtures thereof) of a particular amino acid (methionine, histidine, arginine, lysine, isoleucine, aspartic acid, tryptophan, threonine and mixtures thereof) or combinations of these stereoisomers or glycine or an organic base such as but not limited to imidazole, may be present in the pharmaceutical compositions of the invention so long as the particular amino acid or organic base is present either in its free base form or its salt form. In one embodiment the L-stereoisomer of an amino acid is used. In one embodiment the D-stereoisomer is used. Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be obtained by adding methionine such that the ratio of methionine added to methionine residues ranges from about 1:1 to about 1000:1, such as 10:1 to about 100:1.

In a further embodiment of the invention the composition further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active protein therein. Stabilizing agents of particular interest to the present invention include, but are not limited to, methionine and EDTA, which protect the protein against methionine oxidation, and a nonionic surfactant, which protects the protein against aggregation associated with freeze-thawing or mechanical shearing.

In a further embodiment of the invention the composition further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl serine, phosphatidyl choline, phosphatidyl ethanolamine, phosphatidyl inositol, diphosphatidyl glycerol and sphingomyelin), derivates of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyransoide), sphingoglycolipids (e.g. ceramides, gangliosides), dodecylphosphocholine, hen egg lysolecithin, fusidic acid derivatives—(e.g. sodium tauro-dihydrofusidate etc.), long-chain fatty acids and salts thereof C₆-C₁₂ (e.g. oleic acid and caprylic acid), acylcarnitines and derivatives, N-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (e.g. Dodecyl β-D-glucopyranoside), poloxamines (e.g. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the pharmaceutical composition of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical composition of the present invention.

Pharmaceutical compositions containing a Modified analogue according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the Modified analogue, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the composition of solids, semisolids, powder and solutions for pulmonary administration of Modified analogue, using, for example a metered dose inhaler, dry powder inhaler and a nebulizer, all being devices well known to those skilled in the art.

Compositions of the current invention are specifically useful in the composition of controlled, sustained, protracting, retarded, and slow release drug delivery systems. More specifically, but not limited to, compositions are useful in composition of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the Modified analogue in the form of a nasal or pulmonal spray. As a still further option, the pharmaceutical compositions containing the Modified analogue of the invention can also be adapted to transdermal administration, e.g. by needle-free injection or from a patch, optionally an iontophoretic patch, or transmucosal, e.g. buccal, administration.

The term “stabilized composition” refers to a composition with increased physical stability, increased chemical stability or increased physical and chemical stability.

The term “physical stability” of the protein composition as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein compositions is evaluated by means of visual inspection and/or turbidity measurements after exposing the composition filled in suitable containers (e.g. cartridges or vials) to mechanical/physical stress (e.g. agitation) at different temperatures for various time periods. Visual inspection of the compositions is performed in a sharp focused light with a dark background. The turbidity of the composition is characterized by a visual score ranking the degree of turbidity for instance on a scale from 0 to 3 (a composition showing no turbidity corresponds to a visual score 0, and a composition showing visual turbidity in daylight corresponds to visual score 3). A composition is classified physical unstable with respect to protein aggregation, when it shows visual turbidity in daylight. Alternatively, the turbidity of the composition can be evaluated by simple turbidity measurements well-known to the skilled person. Physical stability of the aqueous protein compositions can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein composition as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein composition can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized composition” refers to a composition with increased physical stability, increased chemical stability or increased physical and chemical stability. In general, a composition must be stable during use and storage (in compliance with recommended use and storage conditions) until the expiration date is reached.

In one embodiment of the invention the pharmaceutical composition comprising the Modified analogue is stable for more than 6 weeks of usage and for more than 3 years of storage.

In another embodiment of the invention the pharmaceutical composition comprising the modified analogue is stable for more than 4 weeks of usage and for more than 3 years of storage.

In a further embodiment of the invention the pharmaceutical composition comprising the modified analogue is stable for more than 4 weeks of usage and for more than two years of storage.

In an even further embodiment of the invention the pharmaceutical composition comprising the Modified analogue is stable for more than 2 weeks of usage and for more than two years of storage.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

### EXAMPLES

The following examples and general procedures refer to intermediate compounds and final products identified in the structural specification and in the synthesis schemes. The preparation of the compounds of the present invention is described in detail using the following examples, but the chemical reactions described are disclosed in terms of their general applicability to the preparation of selected branched polymers of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents. All reagents were of standard grade as supplied from Aldrich, Sigma, etc. Proton and carbon nuclear magnetic resonance (¹H and ¹³C NMR) were recorded on a Bruker NMR apparatus, with chemical shift (δ) reported down field from tetramethylsilane.

LC-MS mass spectra were obtained using apparatus and setup conditions as follows:

### LCMS (Method A)

Hewlett Packard series 1100 G1312A Bin Pump
Hewlett Packard series 1100 Column compartment
Hewlett Packard series 1100 G13 15A DAD diode array detector
Hewlett Packard series 1100 MSD

The instrument was controlled by HP Chemstation software.

The HPLC pump was connected to two eluent reservoirs containing:

A: 0.01% TFA in water

B: 0.01% TFA in acetonitrile

The analysis was performed at 40° C. by injecting an appropriate volume of the sample (preferably 1 μL) onto the column, which was eluted with a gradient of acetonitrile.

The HPLC conditions, detector settings and mass spectrometer settings used are given in the following table.

| | |
|---|---|
| Column | Waters Xterra MS C-18 × 3 mm id |
| Gradient | 10%-100% acetonitrile lineary during 7.5 min at 1.0 ml/min |
| Detection | UV: 210 nm (analog output from DAD) |
| MS | Ionisation mode: API-ES |
| | Scan 100-1000 amu step 0.1 amu |

Some of the NMR data shown in the following examples are only selected data.

### LCMS (Method B)

The following instrumentation is used:
Hewlett Packard series 1100 G1312A Bin Pump
Hewlett Packard series 1100 G13 15A DAD diode array detector
Sciex3000 triplequadropole mass spectrometer
Gilson 215 micro injector
Sedex55 evaporative light scattering detector

Pumps and detectors are controlled by MassChrom 1.1.1 software running on a MacIntosh G3 computer. Gilson Unipoint Version 1.90 controls the auto-injector.

The HPLC pump is connected to two eluent reservoirs containing:

A: 0.01% TFA in water

B: 0.01% TFA in acetonitrile

The analysis is performed at room temperature by injecting an appropriate volume of the sample (preferably 10 μl) onto the column, which is eluted, with a gradient of acetonitrile. The eluate from the column passed through the UV detector to meet a flow splitter, which passed approximately 30 μl/min ( 1/50) through to the API Turbo ion-spray interface of API 3000 spectrometer. The remaining 1.48 ml/min ( 49/50) is passed through to the ELS detector.

The HPLC conditions, detector settings and mass spectrometer settings used are giving in the following table.

| | |
|---|---|
| Column | Waters X-Terra C18, 5μ, 50 mm × 3 mm id |
| Gradient | 5%-90% acetonitrile linearly during 7.5 min at 1.5 ml/min |
| Detection | 210 nm (analogue output from DAD) |
| MS | ionisation mode API Turbo ion-spray |
| | Scan 100-1000 amu step 0.1 amu |
| ELS | Gain 8 and 40° C. |

MALDI-TOF spectroscopy was performed on a Brucker Daltonics Autoflex apparatus, according to the procedure described by Metzger et al. Fresenius 3. Anal. Chem. (1994) 349 473. Matrix was made by dissolving 3-aminoquinoline (10 mg) in MeOH:H₂O (1 ml, 10:90). Samples were applied to the target in a concentration of 80-800 pmoles/μl (≈0.1-1 mg/ml) as aqueous solutions in a ratio with matrix of 1:1. The samples were dried under a stream of N2. Samples were analyzed in linear mode.

In the examples the following terms are intended to have the following, general meanings:

### Abbreviations

AcOEt: ethylacetate.
BSA: Bovine Serum Albumine
DCM: dichloromethane, methylenechloride
DIEA diisopropylethylamine
DMF: N,N-dimethylformamide
Gal-UDP: Uridine 5′-diphospho-D-galactose (Disodium salt)
GO: galactose oxidase (EC 1.1.3.9)
β1,4-galT: β1,4-galactosyl transferase (EC 2.4.1.22)
GlcNAc-UM: 4-methylumbelliferyl-2-acetamido-2-deoxy-β-D-glucopyranoside
Galβ1→4GlcNAc-UM: 4-methylumbelliferyl N-acetyl lactosaminide
α1,3-galT: α1,3-galactosyltransferase (EC 2.4.1.90) (recombinant bovine enzyme expressed in E. coli)
MeOH: methanol
NMP: N-methyl-2-pyrrolidinone
Oxgal-UDP: Uridine 5′-diphospho-6-aldehydro-D-galactose
TEA: triethylamine
TFA: trifluoroacetic acid
THF: tetrahydrofuran
Ts: p-toluenesulfonyl
TsCl: p-toluenesulfonylchloride

The following non-limiting examples illustrate the synthesis of donor sugar nucleotides, acceptor sugar (as models for glycoproteins), chemoenzymatic protocols for saccharide assembly in aqueous solution, and examples for post enzymatic reactions with preferred moieties.

### Example 1

### 1,2:3,4-Di-O-isopropyliden-6-O-tosyl-D-galactopyranose

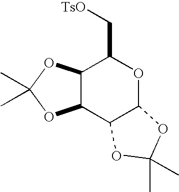

1,2:3,4-Di-O-isopropylidene-D-galactopyranose (1) (12.85 g; 49.7 mmol) was dissolved in dry pyridine (17 ml), and tosylchloride (11.38 g; 59.7 mmol) was added in small portions. The clear yellow solution was stirred at room temperature over night. The reaction mixture was then poured over crushed ice, separating the product as a yellow oil which slowly solidified. The solid was collected by filtration, and recrystallized from hexane to give the title material as fine white crystals. The powder was dried in a vacuum oven overnight. Yield: 16.48 g (80%). ¹H-NMR (400 MHz; CDCl₃): δ 1.28 ppm (s, 3H); 1.32 (s, 3H); 1.35 (s, 3H); 1.50 (s, 3H); 2.43 (s, 3H); 4.07 (m, 2H); 4.20 (m, 2H); 4.30 (dd, 1H); 4.58 (dd, 1H); 5.45 (d, 1H); 7.32 (d, 2H); 7.90 (d, 2H). ¹³C-NMR (400 MHz; CDCl₃): δ 21.64 ppm; 24.35; 24.92; 25.81; 25.98; 65.87; 68.18; 70.37; 70.40; 70.52; 96.13; 108.95; 109.58; 128.14; 129.75; 132.82; 144.75. LC-MS (Method A): Rt=4.04 min. m/e=437 (M+22)⁺;

### Example 2

### 1,2:3,4-Di-O-isopropyliden-6-azido-6-deoxy-D-galactopyranose

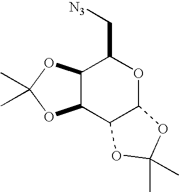

1,2:3,4-Di-O-isopropyliden-6-O-tosyl-D-galactopyranose (5.00 g; 12.6 mmol) was dissolved in DMF (50 ml). Sodium azide (2.35 g; 36.2 mmol) and water (5 ml) were added, and the mixture was heated to 120° C. for 4 days. The reaction was at this point 20% from completion. Therefore additional sodium azide (2.35 g; 36.2 mmol) was added and heating was continued for 8 hours. The reaction mixture was cooled and filtered. The filtrate was reduced to 1/10 of the original volume and then partitioned between ethyl acetate and water. The water phase was separated and extracted once with ethyl acetate. The combined organic extracts were dried (Na2SO4) and the solvent was evaporated. The residual clear oil was re-dissolved in acetonitril and evaporated to dryness to remove residual water. Yield: 3.65 g—oil containing 10 mol % DMF according to H-NMR. ¹H-NMR (400 MHz; CDCl₃): δ 1.35 ppm (ds, 6H); 1.45 (s, 3H); 1.52 (s, 3H); 3.34 (dd, 1H); 3.51 (dd, 1H); 3.90 (m, 1H); 4.18 (dd, 1H); 4.32 (dd, 1H); 4.62 (dd, 1H); 5.55 (d, 1H). ¹³C-NMR (400 MHz; CDCl₃): δ 24.41 ppm; 24.88; 25.94; 26.03; 50.67; 67.00; 70.38; 70.79; 71.16; 96.34; 108.80; 109.61.

### Example 3

### 6-Azido-6-deoxy-D-galactopyranose

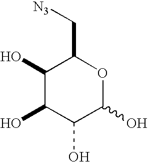

1,2:3,4-Di-O-isopropyliden-6-azido-6-deoxy-D-galactopyranose (2.0 g; 7.01 mmol) was dissolved in 60% TFA-water (100 ml) and the mixture was heated at 50° C. for 3 h. The solution was then evaporated to give a sticky yellow oil. The oil was repeatedly (3×) redissolved in acetonitril and evaporated to dryness in order to remove all water. Yield: 1.45 g (100%). ¹H-NMR (400 MHz; D₂O for the 6:4-mixture of α and β anomers): δ 3.30 ppm (m, 2H); 3.5 (dd, 1H); 3.67 (m, 2H); 3.75+4.10 (double multiplet, 1H); 4.45 (d, H1β); 5.12 (d, H1α). ¹³C-NMR (400 MHz; D₂O for the 6:4-mixture of α and β anomers): δ 49.87 ppm; 50.03; 67.31; 68.04; 68.13; 68.23; 68.76; 70.80; 71.79; 72.57; 91.49; 95.57.

### Example 4

### 6-Azido-6-deoxy-D-galactopyranosyl-1(α,β)-uridinyldiphosphate

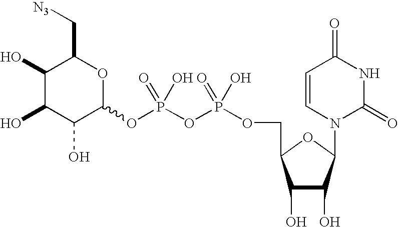

This compound was made analogous to the procedures described in T. Uchiyama and O. Hindsgaul, J. Carbohydrate Chemistry, 17(8), 1181-1190 (1998). 6-Azido-6-deoxy-D-galactopyranose (320 mg, 1.58 mmol) was dissolved in dry pyridine (10 ml), and added trimethylsilylchloride (1.14 ml, 9.48 mmol), which lead to initial precipitation. The mixture was stirred for 1 h on an icebath, then partitioned between water (5 ml) and petrolether (30 ml). The organic phase was washed with water (4×5 ml) and dried over anhydrous sodium sulfate. The solvent was removed and the residual oil was dissolved in dichloromethane (5 ml). The mixture was cooled on an icebath and trimethylsilyliodide (200 ul, 1.4 mmol) was added, leading to immediate formation of a brown colored solution. The mixture was stirred on an icebath for 10 min, then at room temperature for 20 min. Solid tetrabutylammonium uridindiphosphate (508 mg, 0.57 mmol) was added. The darkbrown mixture was stirred at ambient temperature overnight. Solid tetrabutylammonium fluoride (1.48 g, 5.68 mmol) was added followed by 25% aqueous ammonia (150 ul). The mixture was stirred at room temperature for 90 min, to give a clear fainted yellow solution. Solvent was removed by rotary evaporation, and the residue was suspended in 50 mM Tris buffer (40 ml, pH 8.0). Some insoluble material was at this point removed by extraction with dichloromethane and by filtration. The aqueous buffer solution was then added alkaline phosphatase (1100 U), and left at rt for 16 h.

5 ml of this solution (⅛ of total volume) was purified on a preparative HPLC C18-column (20 cm×2 cm i.d.), which was eluted with a gradient of 0-60% acetonitril in 40 mM triethylamin-acetic acid, pH 6.0 over 1 hour, at a flow of 10 ml/min, while monitoring at Abs_{276 nm}. Samples containing product were pooled and freeze dried, to give the title material as its triethylammonium salt.

Yield: 85.3 mg. ¹H-NMR (CDCl₃): δ; 5.78 (αH1); 5.13 (βH1). α/β ratio: 4:6.

MALDI-TOF (3-aminoquinoline matrix): m/e=611.49 (M+Na).

The purified triethylammonium salt was dissolved in water (5 ml) and passed through Dowex X50 (Na⁺ form). Fractions were spotted on a TLC plate under UV light. Fractions containing compound were pooled and lyophilized to give 55.5 mg of title material as its sodium salt.

### Example 5

### 1,2:3,4-Di-O-isopropyliden-6-O-propagyl-D-galactopyranose

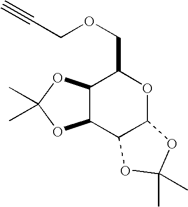

Sodium hydride (24.1 g, 60% oil dispersion, 0.7 mol) was washed trice with petrol ether, and then resuspended in dry THF (100 ml). A solution of 1,2:3,4-di-O-isopropyliden-D-galactopyranose (23.5 g, 90.3 mmol) in dry THF (100 ml) was added dropwise over 20 min. The reaction mixture was stirred at ambient temperature for 1 h. The reaction mixture was cooled on an icebath, and neat propagylbromid (39.96 g, 335 mmol) was then added dropwise over 20 min. The cream colored mixture was then stirred at ambient temperature overnight. The reaction mixture was filtered, and the filtrate was acidified with acetic acid (20 ml). The filtrate was then evaporated to dryness, and the residual dissolved in ethylacetate and washed twice with saturated aqueous sodium carbonate solution and once with brine. The organic phase was then dried with anhydrous sodium sulfate, and evaporated to dryness, to give a dark brown oil which started to crystallize. The semicrystalline residue was dissolved in a minimum of dichloromethane, and added to hot petrol ether (60-80° C. boiling range) containing decolorizing carbon. The mixture was boiled for 10 min., then filtered into a dry conical flask. Upon cooling on a dry-ice-acetone bath, a oil precipitated out, which after a while of vigous stirring turned into a off-white powder. Yield: 13.02 g (48%). ¹H-NMR (CDCl₃): δ 1.33 ppm (s, 3H); 1.35 (s, 3H); 1.46 (s, 3H); 1.54 (s, 3H); 2.42 (s, 1H); 3.72 (ddd, 2H); 3.98 (dt, 1H); 4.24 (dd, 2H); 4.31 (m, 1H); 4.61 (dd, 1H); 5.53 (d, 1H). ¹³C-NMR (CDCl₃): 24.86 ppm; 25.32; 26.37; 26.45; 58.91; 67.14; 69.09; 70.86; 71.69; 71.58; 74.97; 80.04; 96.75; 109.01; 109.74.

### Example 6

### 6-O-Propagyl-D-galactopyranose

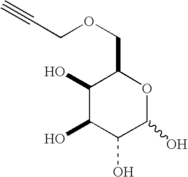

1,2:3,4-Di-O-isopropyliden-6-O-propagyl-D-galactopyranose (4.80 g, 16.1 mmol) was suspended in water (100 ml). Dowex 50 X8 resin (5 g) was added to the suspension and the mixture was heated to 80° C. (internal temperature) on a oil bath overnight. The mixture was filtered, and the clear yellow filtrate was taken to dryness at a low waterbath temperature (<40° C.). The residual oil was evaporated twice from dry acetonitril. Yield: 3.45 g (98%).

¹H-NMR (CDCl₃): δ 2.82 ppm (s, 1H); 3.38 (dd, 1H); 3.51 (dd, 1H); 3.55-3.85 (m, 5H); 4.10-4.21 (m, 3H); 4.45 (d, THE); 5.12 (d, αH1); α/β ratio: 1:2.

¹³C-NMR (CDCl₃, α/β mixture): δ 57.21 ppm; 57.33; 67.35; 67.77; 68.07; 68.13; 68.43; 68.59; 70.87; 72.41; 75.25; 78.29; 91.40; 95.50.

### Example 7

### 6-O-Propagyl-D-galactopyranosyl-1(α,β)-uridinyldiphosphate

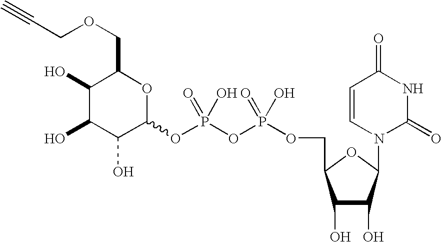

This material may be prepared from 6-O-propagyl-D-galactopyranose and uridine-5′-monophosphomorpholidate as described for 6-azido-6-deoxy-D-galactopyranosyl-1(α/β)-uridinyldiphosphate, or alternatively as described below.

### Example 8

### 2,3,4-Tri-O-acetyl-6-O-propagyl-D-galactopyranose

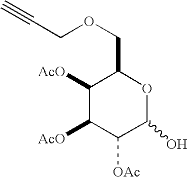

6-O-propagyl-D-galactopyranose (2.90 g, 13.3 mmol) was dissolved in pyridin (50 ml) and acetic anhydride (25 ml) was added. The mixture was stirred at room temperature for 3 h. Solvent was removed by rotary evaporation. The brown residue was evaporated twice from ethanol and once from toluene. The product was dried overnight in a vacuum oven (40° C.). The material was then dissolved in THF (30 ml) and added benzylamine (2.14 g; 20 mmol). The mixture was stirred at ambient temperature for 16 h. The mixture was diluted with water (50 ml) and extracted trice with dichloromethane. The combined organic extracts were washed once with 1N aqueous HCl, once with saturated aqueous sodium bicarbonate once with brine and then dried with sodium sulfate. The solvent was removed by rotary evaporation, and the brown oil was purified by silica gel chromatography using 40% ethylacetate in heptane. Pure fractions (Rf=0.2) were pooled and taken to dryness to give a yellow oil which solidified. The solid was dried in vacuo overnight.

¹H-NMR (CDCl₃, α/β mixture): δ 1.99 ppm (s, 3H); 2.20 (s, 3H); 2.29 (s, 3H); 2.42 (t, 1H); 3.56 (d, 2H); 4.31 (dd, 2H); 4.47 (t, 1H); 5.17 (dd, 1H); 5.41 (dd, 1H); 5.47 (d, 1H); 5.52 (bs, 1H).

¹³C-NMR (CDCl₃, α/β mixture, selected peaks): α/β δ 21.10 ppm; 21.14; 58.95; 67.75; 67.79; 68.72; 69.27; 75.62; 79.29; 91.08; 170.43; 170.68; 170.81.

### Example 9

### 2,3,4-tri-O-acetyl-6-O-propagyl-D-galactopyranosyl cyanoethyl N,N-diisopropyl phosphoroamidite

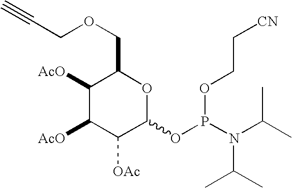

2,3,4-Tri-O-acetyl-6-O-propagyl-D-galactopyranose (400 mg, 1.16 mmol) was dissolved in dry acetonitril and evaporated to dryness. The dried residue was dissolved in dry dichloromethane and cooled on an icebath. Diisopropylethylamine (405 ul, 2.32 mmol) and cyanoethyl-N,N-diisopropyl phosphoroamidylchloride (358 mg, 1.51 mmol) was added and the mixture was stirred for 30 min at 0° C. Additional diisopropylethylamine (405 ul, 2.32 mmol) and cyanoethyl-N,N-diisopropyl phosphoroamidylchloride (358 mg, 1.51 mmol) was added and the mixture was stirred for a further 30 min at 0° C. The mixture was diluted with dichloromethane and washed twice with 1M aqueous sodium carbonate solution and once with brine. The organic solution was dried with anhydrous sodium sulfate and taken to dryness. Yield: 625 mg (100%).

³¹P-NMR (CDCl₃, α/β mixture): 6150.26 ppm; 152.76 ppm.

### Example 10

### 6-O-propagyl-D-galactopyranosyl phosphate triethylamine salt

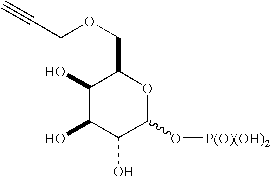

2,3,4-tri-O-acetyl-6-O-propagyl-D-galactopyranosyl cyanoethyl N,N-diisopropyl phosphoroamidite (630 mg; 1.16 mmol) was dissolved in dry acetonitril (4 ml). 3-Hydroxypropionitril (165 mg; 2.32 mmol) and tetrazol (0.45 M acetonitril solution, 5.54 mmol). The mixture was stirred at room temperature for 2 h at which point ³¹P-NMR indicated complete conversion (new signal formed at 138 ppm). The mixture was then added 70% aq. tBuOOH solution (600 ul) and stirred at rt for 30 min. The mixture was taken to dryness and redissolved in dichloromethane (20 ml) and washed with saturated sodium carbonate solution, water and brine. The organic solution was then dried with sodium sulfate. Solvent was removed on a rotary evaporator, to give a clear yellow oil. Yield: 884 mg. The oil was redissolved in freshly prepared 1M NaOMe in methanol (10 ml) and stirred at rt for 1 h. Solvent was removed by rotary evaporation, and the residue dissolved in water (10 ml). The aqueous solution was neutralized with Dowex 50×2 resin to pH 2.4 and quickly back adjusted to pH 7.00 by addition of diluted NaOH solution. The mixture was filtered, and water was removed by rotary evaporation (bath temperature was kept below 30° C.). The yellow residue was triturated twice with acetonitril and the solutions discharged. Sodium ions were replaced by HNEt3+ ions by passing the residue through a 10 cm³ Dowex X50 column (NEt3-form). 2 ml fractions were collected and spotted by 10% anisaldehyde, 5% H2SO4/ethanol on a TLC plate.

The fractions were pooled and evaporated to dryness (water bath temperature was kept below 20° C.) to give a yellow oil. This could be dissolved in acetonitril, and was thus stripped twice (water bath temperature was kept below 20° C.), to give a clear yellow oil. HNEt₃ integration vs. terminal alkyne or anomeric proton showed a ratio of 3.88 HNEts pr. monosaccharide. From this a apparent molecular weight of 633.6 g/mol was calculated. Yield: 506 mg. ¹H-NMR (D₂O): δ 2.79 ppm (s, 1H); 3.65-3.91 (m, 6H); 3.90 (d, 1H); 4.16 (d, 1H); 5.40 (dd, 1H). ³¹P-NMR (D₂O): δ 0.25 ppm (d, 1P).

### Example 11

### 6-O-propagyl-D-galactopyranosyl-1-uridinyldiphosphate

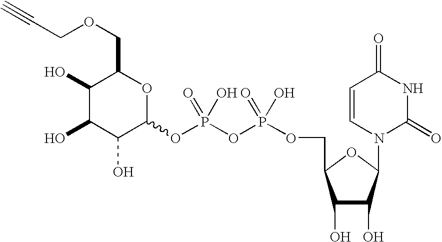

6-O-Propagyl-D-galactopyranosyl phosphate triethylamine salt (250 mg; 0.37 mmol) was dissolved in dry pyridine (2 ml). Trioctylamine (133 mg; 0.37 mmol) was added and the mixture was evaporated to dryness. The residue was dissolved in pyridin (2 ml). Tho the clear solution was added uridine-5′-monophosphomorpholidate (207 mg; 0.30 mmol) followed by a solution of tetrazol in acetonitril (2.7 ml, 0.45 M). The clear yellow solution was stirred at room temperature for 16 h. The reaction mixture was diluted with water (2 ml), and triethyl amine (200 ul) was added to slightly basic pH. Solvent was then evaporated (water bath temperature was kept below 25° C.) and the residue was stripped from water. The residue was dissolved in water (5 ml), and purified by directly loading onto a preparative RP18 HPLC column (20 cm×2 cm i.d.), which was eluted with a gradient of 0-60% acetonitril in 40 mM triethylamin-acetic acid, pH 6.0 over 1 hour, at a flow of 10 ml/min, while monitoring at Abs_{276 nm}. The product eluted at approximately 19-20 min. Samples containing product were pooled and freeze dried, to give a residual oil. ³¹P-NMR (D₂O, anomeric mixture): δ-11.94 ppm; −10.39; −9.94. ¹H-NMR (D₂O, anomeric protons) 5.48 ppm (dd, α-anomer); 5.38 (dd, β-anomer). MALDI-TOF (3-aminoquinoline matrix): m/e=604.76. The purified triethylammonium salt was dissolved in water (5 ml) and passed through Dowex X50 (Na⁺ form). Fractions were spotted on a TLC plate under UV light. Fractions containing compound were pooled and lyophilized to give 55.5 mg of title material as its sodium salt.

### Example 12

### β-1-Phenyloxyethyl N-acetylglycosamineoside (acceptor sugar-model of sialidase/galactosidase trimmed glycoprotein)

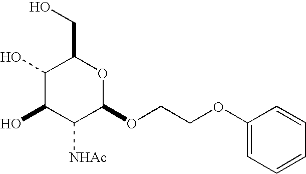

N-Acetylglycosamine (5.00 g; 22.6 mmol) was suspended in acetonitril (50 ml) and phenoxyethanol (8.45 ml; 67.8 mmol) and borontrifluoride etherate (472 ul; 3.7 mmol) was added. The mixture was stirred at 85° C. for 16 h, then cooled to room temperature. Solvent was removed by evacuation, in vacuo, and the residue purified by silica gel chromatography using an eluent of dichloromethane-methanol (20:1). Two fractions containing respectively the α- (fast eluate, 2.58 g, 33%) and the β-anomer (slow eluate, 440 mg; 6%) are collected. The β-anomer is recrystallized twice from acetone to give 140 mg of pure title material. ¹H-NMR (400 MHz; D₂O): 1.83 ppm (s, 3H); 3.21 (m, 1H), 3.27 (m, 1H); 3.40 (t, 1H); 3.55 (m, 2H); 3.77 (m, 1H); 3.87 (m, 1H); 4.10 (m, 4H); 4.47 (d, 1H); 6.99 (m, 3H); 7.35 (m, 2H).

### Example 13

### 2-Phenoxyethoxy N-acetyl-lactosamine

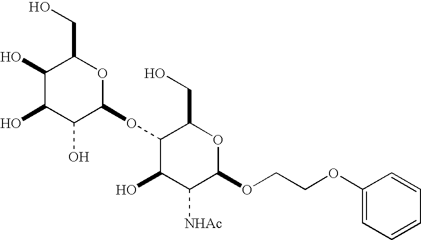

The reaction was run according to Uchiyama and Hindsgaul J. Carbohydr. Chem. 17, 1181, 1998

Solutions of all the reagents were made in cacodylate buffer 100 mM pH 7.5 containing 5 mM manganese chloride.
galactose-UDP (UDP-Gal) (Sigma U-4500): 11 mg/ml
2-phenoxyethoxy N-acetyl-β-D-glucopyranoside: 0.51 mg/ml
Bovine galactosyltransferase (Sigma G5507, 3.9 U/mg solid)): 10 U/ml

2-phenoxyethoxy N-acetyl-β-D-glucopyranoside solution (1.2 ml, 1.8 μmol) was added to the Gal-UDP solution (400 μl, 7.2 μmol), followed by the β1,4 galactosyltransferase solution (100 μl, 1 U) and the alkaline phosphatase (Sigma P-3681) (1 μl, 50 U). The reaction was run at ambient temperature.

The reaction was followed by TLC (CH₂Cl₂/MeOH/H₂O (40:10:1), and the product identified by LC-MS LC system: Waters microbondapak-NH₂ 300×3.9 (10μ) Eluent: CH₃CN/H₂O (85:15) 0.9 ml/min Detection at A=214 nm.

None of the gal acceptor was left after 1 h at ambient temperature.

The product was identified by LC-MS (retention time: 10.5 min), signals observed at (M+H)⁺, and (M+Na)⁺.

### Example 14

This example and the following scheme describes the enzymatic coupling of a donor sugar nucleotide with an acceptor sugar substrate.

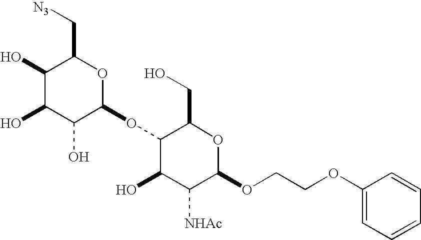

One equivalent of β1-phenyloxyethyl N-acetylglycosamineoside is mixed with 2-10 equivalent of 6-azido-6-deoxy-D-galactopyranosyl-1-uridinyldiphosphate in a 100 mM aqueous sodium cacodylate buffer, pH 7.5 containing 5 mM of MnCl2. Glycosyltransferase (preferably galactosyl transferase or more preferably β1,4-galactosyltransferase (bovine or human)) and alkaline phosphatase is added in sufficient quantity to catalyse disaccharide formation and hydrolyse liberated UDP, respectively, over a 2-36 h time interval at room temperature. When the reaction is complete as judged by TLC the product is purified using standard chromatography techniques.

### Example 15

This example illustrates how disaccharides with non-biogenic handles are reacted further with preferable moieties in aqueous solution to give new modified sugar derivatives.

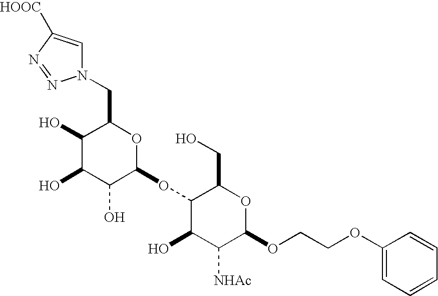

One equivalent of disacharide prepared in example 14 is dissolved in a 1 mM aqueous solution of copper sulphate-sodium ascorbate. 5-20 equivalents of propyne acid is added, and the mixture is stirred at room temperature. Reaction progress is monitored by LC-MS. When the reaction is complete the product is purified using standard chromatography techniques.

### Example 16

### 6-S-Acetyl-1,2:3,4-di-O-isopropyliden-6-thio-D-galactopyranose

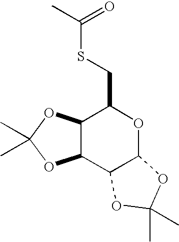

Triphenylphosphine (1.16 g, 4.4 mmol) was dissolved in dry THF (4 ml) and cooled to 0° C. under a nitrogen flow. Diisopropyl azodicarboxylate (0.89 g, 4.4 mmol) was added and a white precipitate formed. More THF (4 ml) was added. After mixing for 20 min at 0° C., a solution of 1,2:3,4-Di-O-isopropylidene-D-galactopyranose (0.57 g, 2.2 mmol) and thioacetic acid (0.34 g, 4.4 mmol) in THF (4 ml) was added dropwise. The solution was allowed to warm to room temperature and stirred for 4 h. Sat. NaHCO₃ (25 ml) and AcOEt (25 ml) were added. The phases were separated and the organic phase was washed with water (25 ml) plus a little sat. NaCl to reduce the emulsion, dried over MgSO₄ and concentrated to yield a brown oil (2.6 g). Flash chromatography of the oil (Silica, 300 ml 3:1 then 1:1 heptane/AcOEt) yielded 587 mg of a brown oil. Another round of chromatography (60 g silica, 5:1 heptane/AcOEt) yielded the desired product as a light orange oil (0.33 g, 47% yield). ¹H-NMR (300 MHz; CDCl₃): δ 1.32 ppm (s, 3H); 1.35 (s, 3H); 1.45 (s, 3H); 1.48 (s, 3H); 2.34 (s, 3H); 3.03 (dd, 1H); 3.16 (dd, 1H); 3.85 (m, 1H); 4.29 (m, 2H); 4.61 (dd, 1H); 5.51 (d, 1H). LCMS (Method A): Rt=3.48 min, m/e=341 (M+23).

### Example 17

### 6-S-Acetyl-6-thio-D-galactopyranosyl-1-uridinyldiphosphate

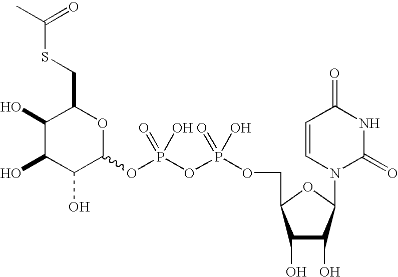

Treatment of 6-S-acetyl-1,2:3,4-di-O-isopropyliden-6-thio-D-galactopyranose with TFA provide 6-S-acetyl-6-thio-D-galactopyranose which can be transformed into the desired donor sugar nucleotide using the method described in Example 4.

### Example 18

### 1,2:3,4-Di-O-isopropyliden-6-thio-D-galactopyranose

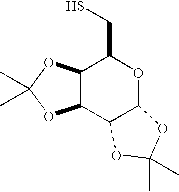

6-S-Acetyl-1,2:3,4-di-O-isopropyliden-6-thio-D-galactopyranose (135 mg, 0.42 mmol) was dissolved in 1 ml MeOH and placed under a flow of nitrogen. A 100 μl aliquot of 30% sodium methoxide in methanol was added. The solution was stirred for 1 h at room temperature and acetic acid (1 ml) was added. The sample was concentrated under vacuum. AcOEt (10 ml) was added, and the solution was washed with water (2×5 ml), dried over MgSO₄, and concentrated under vacuum to yield a colorless oil (112 mg). Flash chromatography of the oil (silica, 4:1 heptane, AcOEt) yielded the desired compound as a colorless oil (76 mg, 65% yield). ¹H-NMR (300 MHz; CDCl₃): δ 1.34 ppm (s, 3H); 1.35 (s, 3H); 1.44 (s, 3H); 1.55 (s, 3H); 1.62 (m, 1H); 2.73 (m, 2H); 3.79, (m, 1H); 4.33 (m, 2H); 4.63 (dd, 1H); 5.54 (d, 1H).

### Example 19

### 1,2:3,4-Di-O-isopropyliden-6-methyldithio-D-galactopyanose

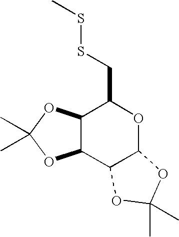

Using a procedure analogous to that described by C. Leriverend and P. Metzner Synthesis, (8), 761-762 (1994), the 1,2:3,4-di-O-isopropyliden-6-thio-D-galactopyanose can be converted to the desired compound.

A solution of SS-Methyl-2-methyldithioperoxypropanoate and diisopropylethylamine in pentane is placed under a nitrogen flow, and 1,2:3,4-di-O-isopropyliden-6-thio-D-galactopyanose is added. After stirring at RT for 1 h, the resulting suspension is filters over Celite, and washed with sat. NaHCO₃ and sat. NaCl. The organic phase is dried over MgSO₄, and concentrated under vacuum. The residue is purified by flash chromatography to yield the desired asymmetric disulfide.

### Example 20

### 6-methyldithio-D-galactopyranosyl-1-uridinyldiphosphate

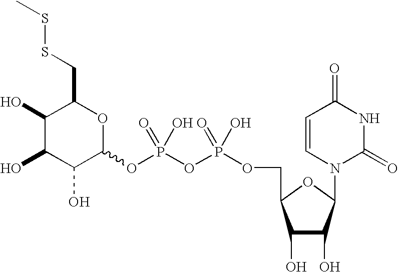

Treatment of 1,2:3,4-di-O-isopropyliden-6-methyldithio-D-galactopyanose with TFA can yield 6-methyldithio-D-galactopyanose which can be transformed into the desired donor sugar nucleotide 6-methyldithio-D-galactopyranosyl-1-uridinyldiphosphate using the method described in example 4.

### Example 21

In these examples, reactions are runned in reaction buffer A, which has the following composition Buffer A: 50 mM MES buffer pH6.6 containing 5 mM MnCl₂ and 1 mg/ml BSA

### Galactose Oxidase Catalyzed Oxidation of gal-UDP:

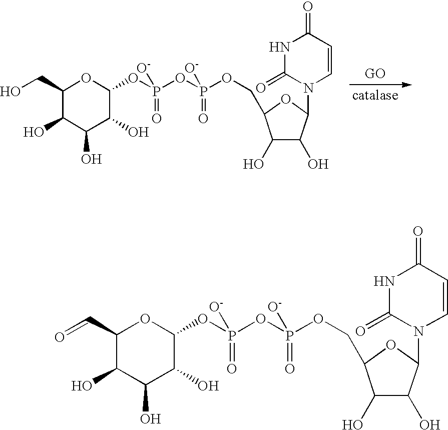

To gal-UDP (sodium salt) (2.4 mg, 4 mM final concentration) in solution in phosphate buffer 25 mM pH 6 (0.97 ml) was added galactose oxidase (Worthington LS4524, 96 U/mg) (1.68 U in 2.5 μl phosphate buffer 25 mM pH6, i.e. final concentration: 1.72 U/ml), followed by catalase (Sigma C-9322, 2350 U/mg solid) (440 U/2.5 μl phosphate buffer 25 mM pH 6). The reaction mixture was incubated at ambient temperature, and followed by HPLC (method 1 described below).

60% aldehyde was formed after 2 h. More galactose oxidase and catalase were added after 2 h (6.70 U GO and 1760 U catalase), and after 3 h30 (3.36 U GO and 880 U catalase). After a total of 5 h reaction time, 97% of the starting material was oxidized to the aldehyde. The reaction mixture was ultra filtered on AmiconUltra cut off 10 kD and used directly in the β1,4-galT transgalactosidation step.

HPLC method 1: according to Ramm et al. J. Chromatography A, 1034 (2004), 139

Column: Reverse phase C18 from YMC, 250×4.6, 5μ

Eluent: A: 40 mM TEA, pH adjusted to 6 by addition of glacial acetic acid

1 ml/min

22° C.

Detection: UV at 214 and 254 nm

Gal-UDP retention time: 12.5 min

0xgal-UDP: 11.5 min

### Example 22

### β1,4-Galactosyl transferase (bovine) catalyzed transfer of 6-aldehydro-gal-UDP onto GlcNAc-UM

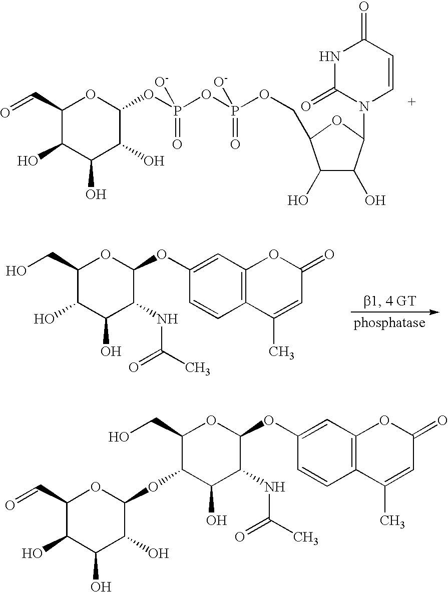

To the aldehyde solution obtained in example 21 (68.3 μl, 0.28 μmol, 3.8 equiv. (2.37 mM final concentration) was added the acceptor GlcNAc-UM (28 μg, 74 nmoles, 0.63 mM final concentration) in solution in Hepes buffer 100 mM pH7.5 containing 5 mM MnCl₂ (45.3 μl). The reaction was started by the addition of β1,4-galT (bovine enzyme, Sigma G5507) in solution in water (4.2 μl of a 100 U/ml solution): (3.5 mU/ml final amount), followed by the addition of alkaline phosphatase (55 U/μl, Sigma P-3681) (0.4 μl, 22 U, 186 mU/ml final amount).

The reaction mixture was incubated at ambient temperature. The reaction was monitored by HPLC method 2 described below:

### HPLC Method 2:

Column: Vydac 218TP53 (Protein and peptide C18) 250×4.6

Eluents: A. H₂O B. CH₃CN

Flow: 1 ml/min

Temperature: 40° C.

Gradient: 2.5 to 100% B over 10 min

Detection: 210 nm, 254 nm Fluo: Exc: 315 nm Em: 375 nm

Oxgal-UDP retention time: 1.40 min

GlcNAc-UM retention time: 4.22 min

Product 4: retention time: 4.13 min

Monitoring of the reaction on HPLC:

As shown above, the reaction ran to completion within less than 24 h.

The product obtained was identified by LCMS (Method B): a signal was detected at m/z=540.3, corresponding to [M+H]⁺ (calc MW=539.5).

### Example 23

### (Human) β1,4-galactosyl transferase catalyzed transfer of 6-aldehydro-gal-UDP onto GlcNAc-UM

The reaction was run as in example 22, except that the recombinant human β1,4-galactosyl transferase (expressed in S. cerevisia, Fluka 90261, 100 U/ml in solution in cacodylate buffer 100 mM pH7.5 containing 5 mM MnCl₂) was used.

The transfer of the 6-aldehydro-galactose was slower than with the bovine enzyme, reaching 16% of product formation after 2 days at ambient temperature.

### Example 24

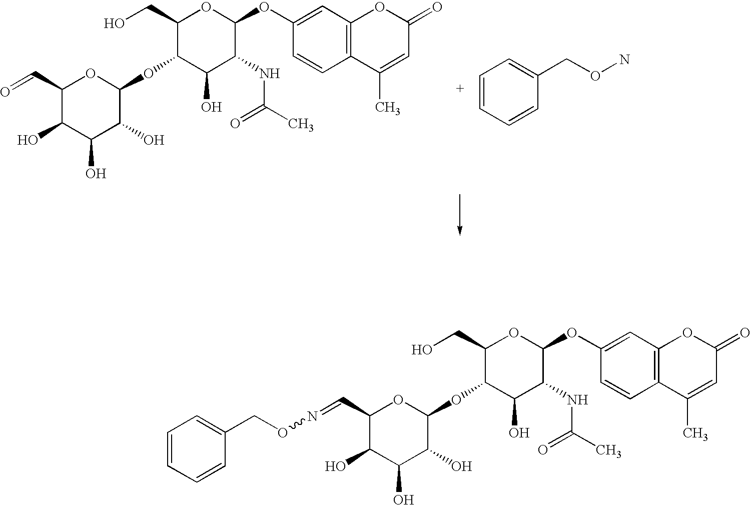

To the reaction mixture obtained in example 22, was added benzylhydroxylamine (100 equivalents in 118 μl Hepes buffer 100 mM pH7.5 containing 5 mM MnCl₂). The reaction was followed by HPLC (HPLC method 2).

The oxime products eluted at retention times 5.53 and 5.65 min (both syn and anti oxime product are formed). The reaction was completed within less than 15 min.

The products obtained were identified by LCMS (Method B): signals were detected at m/z=667.3 and 690.3, corresponding to [M+H]⁺ and [M+Na]⁺ (calc MW=667.4 and 690.4).

### Example 25

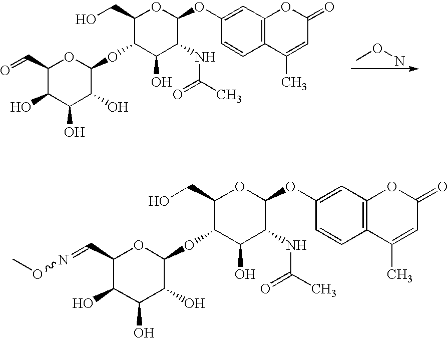

The same procedure as in example 24 was used.

LCMS (Method B) identification: m/z=569.3 and 591.3, corresponding to [M+H]⁺ and [M+Na]⁺ (calc MW=668.5 and 591.5).

### Example 26

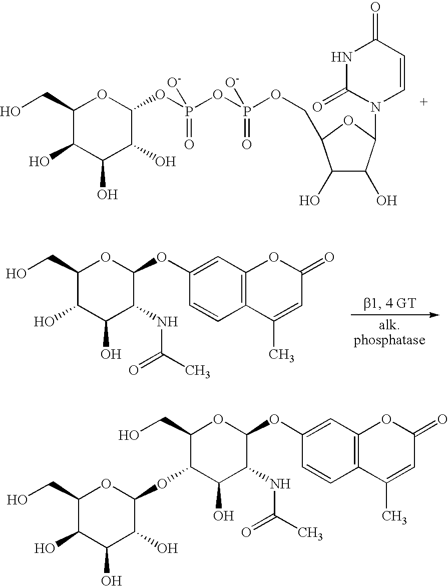

Reaction conditions are slightly modified from Uchiyama and Hindsgaul J. Carbohydr. Chem. 17, 1181 (1998)

To Gal-UDP (22 mg, 3.2 mM final concentration) and GlcNAc-UM (3.4 mg, 1.28 mM final concentration) in solution in cacodylate buffer 100 mM pH7.5 containing 5 mM MnCl₂, was added β1,4-galT (bovine enzyme, Sigma G5507, 250 μl of a 10 U/ml solution in cacodylate buffer 100 mM pH7.5 containing 5 mM MnCl₂, final concentration 0.22 U/ml), followed by alkaline phosphatase 5 μl of a 55 U/μl solution, i.e. final concentration 24.5 U/ml).

The reaction mixture was incubated at ambient temperature, and the reaction was followed by HPLC (HPLC method 2). The reaction mixture became cloudy. The reaction was completed within less than 20 min.

GlcNAc-UM retention time: 4.20 min

Galβ1-4GlcNAc-UM: retention time: 4.14 min

The reaction mixture was filtered, and then purified on a YMC reverse phase C18 250×10 column.

The eluents were: A: H₂O and B: CH₃CN. A gradient from 2 to 60% B was run over 18 min. The product eluted at 19.6 min. A yield of 80% after purification was obtained.

LCMS (Method B) analysis: signals were detected at m/z=542.5 and 564.3, corresponding to [M+H]⁺ and [M+Na]⁺ (calc MW=541.5 and 564.5).

¹H and ¹³C NMR selected chemical shifts of Galβ1-4GlcNAc-UM (400 MHz, D₂O):

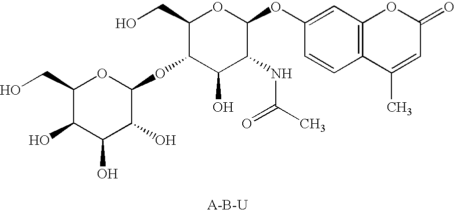

| Residue | H or C | ¹H (ppm) | ¹³C (ppm) |
|---|---|---|---|
| UM | U3 | 6.1 | |
| | UCH₃ | 2.3 | 16.65 |
| | U5 | 7.55(d) | 125.36 |
| | U6 | 6.92(d) | |
| | U8 | 6.85 | |
| GlcNAc | | | |
| | B1 | 5.2(d) | |
| | B2 | | 53.59 |
| | BC═O | | 176.0 |
| | BCH₃ | 2.0 | 20.86 |
| | B6 | | 58.52 |
| Gal | | | |
| | A2 | 4.45(d) | |
| | A6 | | 58.52 |

### Example 27

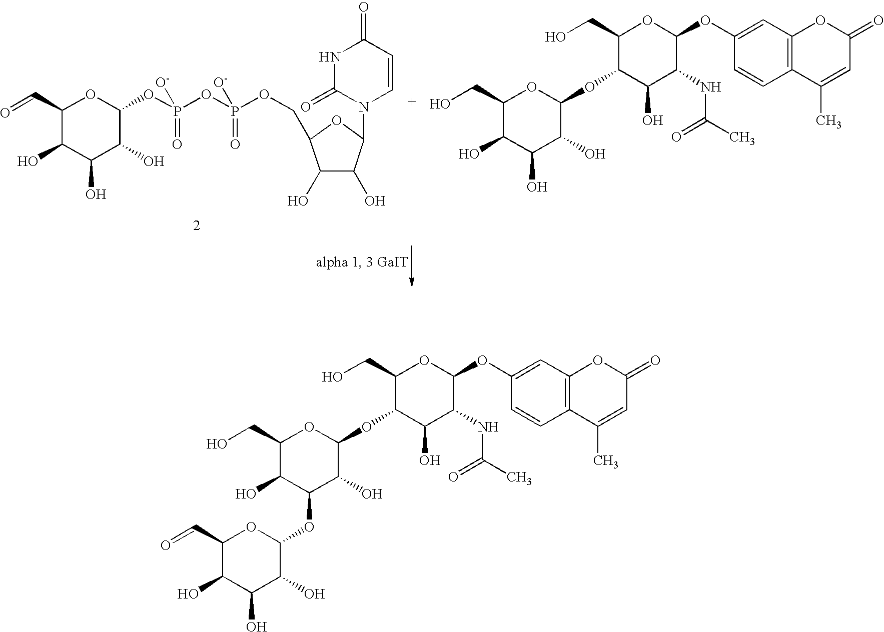

The reaction was run essentially after the procedure described by Stults et al. Glycobiology 9(7), 661 (1999).

The reaction was run in 50 mM MES buffer pH6.6 containing 5 mM MnCl2 and 1 mg/ml BSA (buffer A).

To the aldehyde solution obtained in example 21 (31 μl, 2.5 equivalents, 1 mM final concentration) was added the acceptor Gal-β1,4-GlcNAc-UM (26.5 μg in 60 μl buffer A, 0.4 mM final concentration), followed by buffer A (29.3 μl). The reaction was started by addition of the enzyme α1,3-galT (bovine enzyme, Calbiochem #345647) (0.5 U/ml, 2.4 μl, final concentration 10 mU/ml).

The reaction mixture was incubated at 37° C. The reaction was followed by HPLC (HPLC method 3 described below)

### HPLC Method 3:

Vydac 218TP53 (Protein and peptides C18) 250×4.6

A: H₂O B: CH3CN

1 ml/min

2.5 to 50% B over 10 min

Det: 210, 254 nm Fluo: Exc: 315 nm Em: 375 nm 40 C

Rt Gal-β1,4-GlcNac-UM=5.11 min

Rt oxgal-α1,3-Gal-β1,4-GlcNac-UM=5.02 min

After 2 h reaction time, more enzyme (12 mU) and donor (2.5 equivalents) were added. The same amount of enzyme and donor were added again at 22 h reaction time.

After 39 h reaction time, about 36% of the trisaccharide product was obtained (as judged by H PLC).

Monitoring of the reaction on HPLC:

The product was identified by LC-MS (Method B):

[M+H]⁺ detected at m/z=702.5 Calc: 701.6

### Example 28

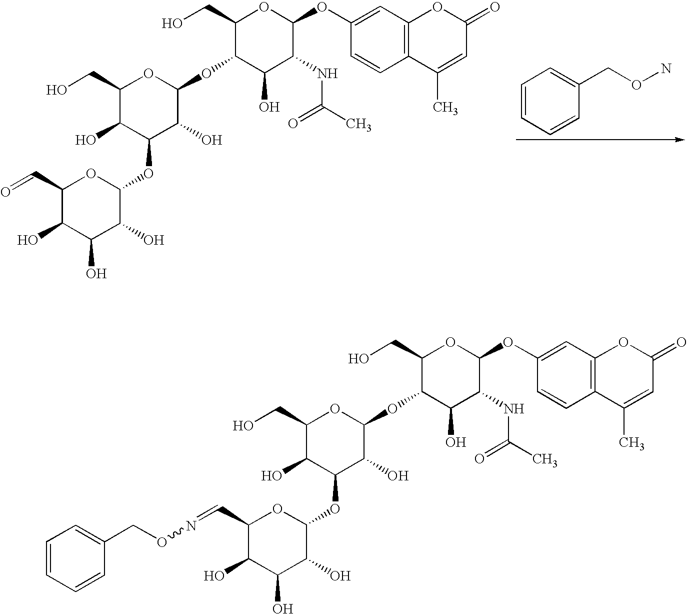

To the product 6-aldehydro-gal-α1,3-gal-β1,4-GlucNAc-UM obtained in example 27 was added benzylhydroxylamine (100 equivalents) in solution in buffer A (mix 1:1 v/v with example 7 reaction mixture). After 15 min at ambient temperature, the reaction mixture was run on HPLC (method 3).

The products (syn and anti forms of the oxime product) were eluting at Rt=7.59 and 7.85 min.

The products were identified by LC-MS (Method B): signals were detected at m/z=807.7 and 829.7, corresponding to [M+H]⁺ and [M+Na]⁺ (calc MW=806.8 and 829.8).

### Example 29

To the product 6-aldehydro-gal-α1,3-gal-β1,4-GlucNAc-UM obtained in example 27 was added methylhydroxylamine (100 equivalents) in solution in buffer A (mix 1:1 v/v with example 7 reaction mixture). After 15 min at ambient temperature, the reaction mixture was run on HPLC (method 3).

The products (syn and anti forms of the oxime product) were eluting at Rt=5.61 and 5.72 min.

The products were identified by LCMS (Method B): signals were detected at m/z=731.3 and 753.5, corresponding to [M+H]⁺ and [M+Na]⁺ (calc MW=730.7 and 753.7).

### Example 30

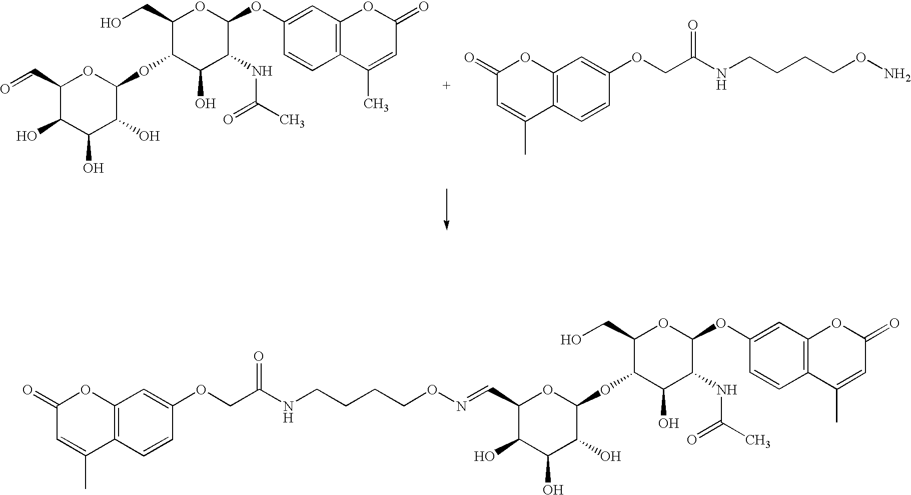

To the product 6-aldehydro-gal-β1,4-GlucNAc-UM obtained in example 22 was added N-(4-aminooxy-butyl)-2-(4-methyl-2-oxo-2H-1-benzopyran-7-yloxy)acetamide (See example 34, 1 equivalent) in solution in acetonitrile (mix 1:2 v/v with example 7 reaction mixture). The reaction mixture was run on HPLC (method 3). After 40 min at ambient temperature, no starting material was left.

The products (syn and anti forms of the oxime product) were eluting at Rt=7.67 and 7.93 min.

The products were identified by LCMS (Method B): signals were detected at m/z=842.8 for each of the new peak, corresponding to [M+H]⁺ (calc MW=841.8).

### Example 31

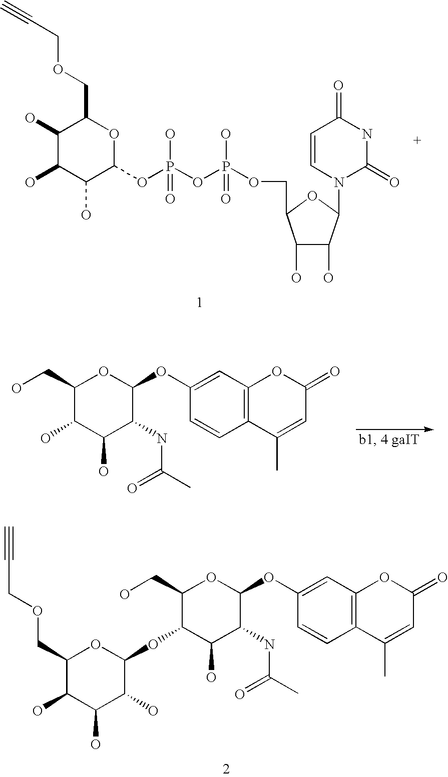

To the alkyne gal-UDP derivative produced in example 11 (389 μg, 0.6 μmole, 4 equivalents) and GlcNAc-UM (56 μg, 0.15 μmole) in solution in Hepes buffer 100 mM pH7.5 containing 5 mM MnCl₂ (117.8 μl) was added alkaline phosphatase (0.8 μl of a 55 U/μl solution in water) and β1,4-galT (bovine enzyme, Sigma G5507, 8.4 μl of a 100 U/ml solution in Hepes buffer 100 mM pH7.5 containing 5 mM MnCl₂).

The reaction mixture was incubated at 30° C., and the reaction was followed by HPLC (HPLC method 2). After 19 h reaction time, more galactosyl transferase was added (8.4 μl of a 100 U/ml solution in Hepes buffer 100 mM pH7.5 containing 5 mM MnCl₂). After 51 h reaction time, 38% of the product 2 was obtained (Rt=4.68 min).

The identity of the product was confirmed by LCMS (Method B): signals were detected at m/z=580.3 and 602.3, corresponding to [M+H]⁺ and [M+Na]⁺ (calc MW=580.6 and 602.6).

### Example 32

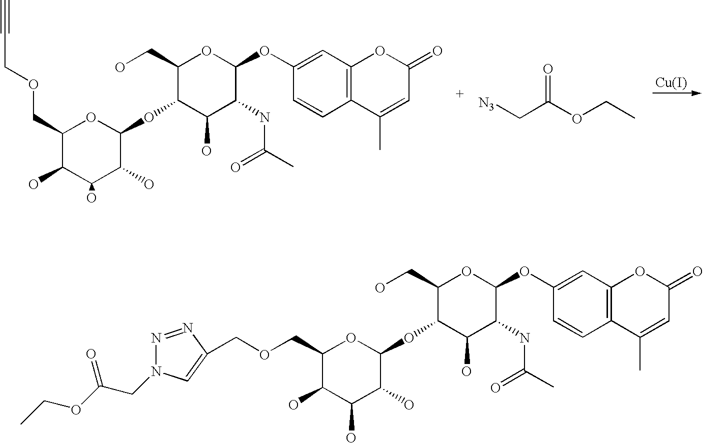

The reaction mixture obtained in example 31 was ultrafiltered (membrane cut off 10 kD). An aliquot (30 μl) was taken out and the azido acetic acid ethyl ester (5 μl of a 1.78 mg/ml solution in (4% 2,6-lutidine:acetonitril (9:1), 10 equivalents) was added.

A solution of copper sulfate and ascorbic acid (respectively 11.9 and 59.5 mM in 2% 2,6-lutidine) was made immediately before addition to the mixture above (5.8 μl, ie 10 equivalents of copper sulfate and 50 equivalents of ascorbic acid).

The reaction was run at ambient temperature and was followed by HPLC (HPLC method 2). The reaction was finished within 2 min, giving a compound with a retention time of 5.18 min on HPLC.

The identity of the product was confirmed by LCMS (Method B): signals were detected at m/z=709.5 and 731.5, corresponding to [M+H]⁺ and [M+Na]⁺ (calc MW=709.7 and 731.7).

### Example 33

### N-(4-tert-butoxycarbonylaminooxybutyl)-2-(4-methyl-2-oxo-2H-chromen-7-yloxy)acetamide

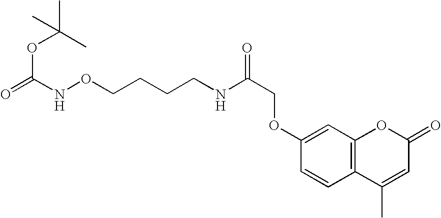

(4-Methyl-2-oxo-2H-chromen-7-yloxy)acetic acid (6 g, 25.6 mmol) was dissolved in DMF (100 ml). N-Ethyl-N′-[3-(dimethylamino)propyl]carbodiimide hydrochloride (6.38 g; 33.3 mmol) and 1-hydroxybenzotriazole (4.5 g; 33.3 mmol) were added and the mixture was stirred for 30 min at room temperature. DIEA (5.7 ml; 33.3 mmol) followed by a solution of O-(4-aminobutyl)-N-tert-butoxycarbonyl hydroxylamine (5.233 g; 25.6 mmol) in DMF (10 ml) washing the vessel with DMF (15 ml). After stirring for 16 h, AcOEt (350 ml) was added and the solution was washed with 5% AcOH (3×300 ml) using sat. NaCl to aid in phase separation, and sat. NaHCO₃ (3×150 ml). The organic phase was dried over MgSO₄ and concentrated under vacuum. AcOEt (75 ml) was added followed by heptane (75 ml) and more AcOEt (25 ml). The resulting precipitate was filtered off and washed with 1:1 AcOEt/heptane. The residue was dried under vacuum to yield 4.2 g. The solid was dissolved in refluxing AcOEt (200 ml) and cooled to yield crystals, which were filtered off and washed with 1:1 AcOEt/heptane, and dried under vacuum to yield white crystals (3.25 g, 30%). ¹H-NMR (400 MHz; CDCl₃) δ 1.47 ppm (s, 9H); 1.69 (m, 4H); 2.41 (s, 3H); 3.42 (q, 2H); 3.88 (t, 2H); 4.55 (s, 2H); 6.18 (s, 1H); 6.73 (br, 1H); 6.86 (s, 1H); 6.90 (m, 1H); 7.21 (s, 1H); 7.55 (d, 1H). LCMS (Method A): Rt=1.56 min. m/z=443 (M+23).

### Example 34

### N-(4-Aminooxybutyl)-2-(4-methyl-2-oxo-2H-chromen-7-yloxy)acetamide (TFA salt)

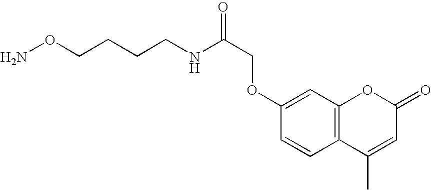

N-(4-tert-butoxycarbonylaminooxybutyl)-2-(4-methyl-2-oxo-2H-chromen-7-yloxy)-acetamide (1 g; 2.38 mmol) was dissolved in TFA (25 ml) and mixed on a rotary evaporator for 30 min at room temperature. The sample was concentrated under vacuum, and residual TFA was removed by adding DCM and removing it under vacuum, then adding diethyl ether and removing it under vacuum, thus producing a white residue (0.8 g). ¹H-NMR (400 MHz; DMSO) δ 1.53 ppm (m, 4H); 2.40 (s, 3H); 3.16 (q, 2H); 3.91 (t, 2H); 4.61 (s, 2H); 6.24 (s, 1H); 6.96 (s, 1H); 7.02 (d, 1H); 7.72 (d, 1H); 8.22 (t-br, 1H); 10.36 (br, 3H). LCMS (Method A): Rt=0.92 min. m/z=321 (M+1).

### Example 35

### 6-{4-[2-(4-Methyl-2-oxo-2H-chromen-7-yloxy)acetylamino]butoxyimino}-6-deoxy-D-galactopyranosyl-1(α)-uridinyldiphosphate (syn/anti mixture)

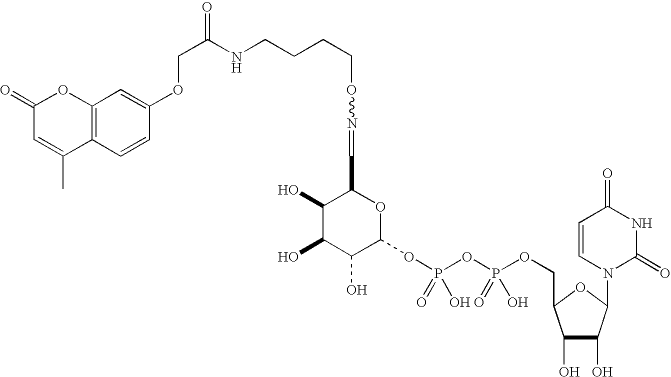

25 mM Na₂HPO₄/NaH₂PO₄ pH 6.1 buffer was used in the following reaction (buffer). UDP-α-D-galactose disodium salt (0.4 g; 0.7 mmol) was dissolved in 24 ml buffer. A solution of catalase (20.61 mg; 41220 units, in 5 ml buffer) was added. A solution of the TFA salt of N-(4-Aminooxybutyl)-2-(4-methyl-2-oxo-2H-chromen-7-yloxy)acetamide (0.60 g, 1.87 mmol, in buffer (14 ml) and acetonitrile (14 ml), and adjusted to pH 6.28 with 1 N NaOH) was added. A solution of galactose oxidase (ca. 10 mg; ca. 1000 units, in buffer (5 ml)) was added. The mixture was allowed to stand at room temperature for 16 h. The enzymes were removed using centrifugal filters with a 10000 MW cut-off. Ca. half of the filtrate was purified using a sep-pak column (10 g, Waters Sep-Pak vac 35 cc, C18, WAT043345). The column was prepared by washing with MeOH (50 ml) and water (50 ml). The filtrate (ca. 30 ml) was added to the column and the eluate was collected. The column was eluted with Milli Q water (4×50 ml), 5% MeOH (2×50 ml), 10% MeOH (2×50 ml) and 20% (1×50 ml). The last water fraction and the first three 5% MeOH fractions were pooled and lyophilized to yield a white solid (52 mg, 8%). The other half of the filtrate was purified in the same manner to yield a white solid (38 mg, 6%). ¹H-NMR (400 MHz; D₂O) δ 1.41 ppm (m, 4H); 2.33 (s, 3H); 3.17 (t-br, 2H); 3.50-4.23 (m, 12H, (theoretical 11H)); 4.59 (s, 2H); 5.53 (m, 1H); 5.69-5.75 (m, 2H); 6.14 (s, 1H); 6.67 (d, 0.20H, Syn-CH); 6.83 (d, 1H), 6.93 (dd, 1H); 7.32 (d, 0.74H, Anti-CH); 7.63 (d, 1H), 7.73 (m, 1H). LCMS (Method B): Rt=2.05 min. m/z=868 (M+1).

### Example (36)

### 1,2:3,4-di-O-isopropyliden-6-(3-Carboxy-4-nitro-phenyldisulfanyl)-6-deoxy-D-galactopyranose

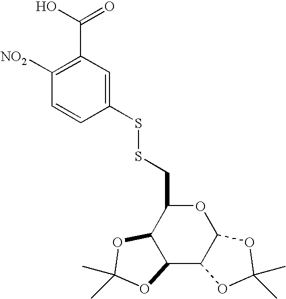

6-S-Acetyl-1,2:3,4-di-O-isopropyliden-6-thio-D-galactopyranose (220 mg; 0.63 mmol) was dissolved in MeOH (1.5 ml) under a flow of nitrogen. A 30% solution of sodium methanolate (0.175 ml, 0.94 mmol) was added, and the reaction was stirred at room temperature and followed with TLC (1:3 AcOEt/heptane). After 1 h, acetic acid (54 μl) was added. After stirring 5 min. at room temperature, 5,5′-dithiobis(2-nitobenzoic acid) (249 mg; 0.63 mmol) was added. The solution became yellowish-orange and was stirred at room temperature for 1.5 h. The sample was concentrated under vacuum. The residue was purified by flash chromatography (silica, 40 mm i.d.×7.5 cm, 7:3 AcOEt/heptane+0.1% AcOH) to yield a white solid (96 mg). The solid was further purified by flash chromatography (silica) by elution first with 7:3 AcOEt/heptane+1% TEA followed by AcOEt followed by AcOEt+5% acetic acid. The appropriate fractions were concentrated under vacuum, and DCM was added and the sample was concentrated again to yield an oil (80 mg; 27%). ¹H-NMR (400 MHz; CDCl₃) δ 1.32 ppm (s, 3H); 1.34 (s, 3H); 1.42 (s, 3H); 1.44 (s, 3H); 2.96-3.08 (m, 2H); 3.99 (m, 1H); 4.25 (dd, 1H); 4.33 (dd, 1H); 4.63 (dd, 1H); 5.54 (d, 1H); 7.76 (dd, 1H); 7.90 (d, 1H); 7.95 (d, 1H).

### Example (37)

### 6-(3-Carboxy-4-nitro-phenyldisulfanyl)-6-deoxy-D-galactopyranose

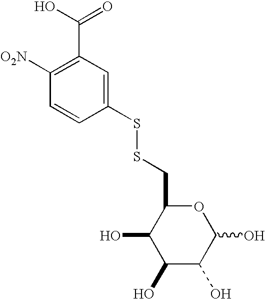

Dowex 50W X2 resin (H⁺ form, 100 mg) was washed with water and added to a solution of 1,2:3,4-di-O-isopropyliden-6-(3-Carboxy-4-nitro-phenyldisulfanyl)-6-deoxy-D-galactopyranose (80 mg; 0.17 mmol) in water (2 ml). The reaction was stirred at 75° C. for 3 h then at room temperature for 16 h, then at 75° C. for 5 h. The resin was filtered off and washed with water (6×1 ml). The filtrate was lyophilized to yield the title compound as a mixture of α and β isomers (48 mg; 61%). LCMS (Method A): Rt=0.90 min. m/z=416 (M+23).

### Example (38)

### 6-(3-Carboxy-4-nitro-phenyldisulfanyl)-6-deoxy-D-galactopyranosyl-1(α/β)-uridinyldiphosphate

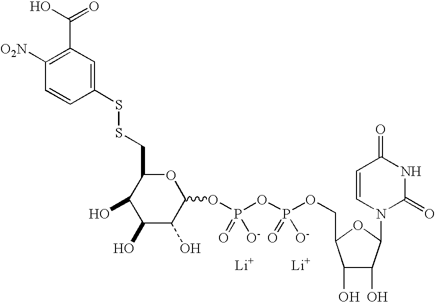

6-(3-Carboxy-4-nitro-phenyldisulfanyl)-6-deoxy-D-galactopyranose can be converted to 6-(3-methoxycarbonyl-4-nitro-phenyldisulfanyl)-6-deoxy-D-galactopyranose using procedures analogous to those described in Hecker, S. J. and Minich, M. L.; J. Org. Chem. 55 (24), 6051-6054 (1990), (e.g. benzyl bromide and NaHCO₃ in DMF). 6-(3-methoxycarbonyl-4-nitro-phenyldisulfanyl)-6-deoxy-D-galactopyranose can be transformed into the title compound via methods analogous to those described in Binch, H.; Stangier, K. and Thiem, Carbohydrate Research, 306, 409-419 (1998) with the exceptions that uridine-5′-monophosphomorpholidate (4-morpholine-N′,N-dicyclohexylcarboxamidine salt) should be used instead of the guanosine-5′-monophosphomorpholidate (4-morpholine-N′,N-dicyclohexylcarboxamidine salt), and the phosphorylation of the galactopyranose should be performed by a procedure analogous to that described in Garcia, B. A. and Bin, D. Y. Org. Lett., 2 (14), 2135-2138 (2000) in order to produce a more advantageous α/β ratio.

### Example (39)

### 2,3,5-Tri-O-acetyl-6-bromo-6-deoxy-L-galactono-1,4-lactone

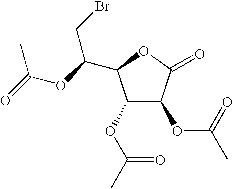

The compound was prepared by the method described in Binch, H.; Stangier, K. and Thiem, Carbohydrate Research, 306, 409-419 (1998) to yield a white solid (8.6 g; 84%). ¹H-NMR (400 MHz; CDCl₃) δ 2.14 ppm (s, 3H); 2.17 (s, 3H); 2.19 (s, 3H); 3.53 (m, 2H), 4.84 (d, 1H); 5.21 (t, 1H), 5.39 (t, 1H); 5.63 (d, 1H). LCMS (Method A): Rt=1.51 min. m/z=367+369 (M+1, M+3).

### Example (40)

### 4-(2-Methyl-1,3-dioxolan-2-yl)phenol

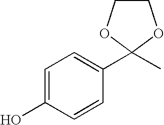

4-Hydroxy acetophenone (15 g, 110 mmol) and imidazole (11.25 g, 165 mmol) were dissolved in DMF (100 ml). tert-Butyldimethylsilyl chloride (24.9 g, 165 mmol) was added, washing with DMF (30 ml). The reaction was stirred under nitrogen for 1.5 h at room temperature. The solvent was removed under vacuum. AcOEt (150 ml) was added and the solution was washed with water and 0.5 N HCl (100 ml each), dried over MgSO₄, and concentrated under vacuum to yield a yellow oil which later crystallized (25.88 g; 94%) ¹H-NMR (300 MHz; CDCl₃) δ 0.23 ppm (s, 6H); 0.99 (s, 9H); 2.55 (s, 3H); 6.87 (d, 2H); 7.88 (d, 2H). LCMS (Method A): Rt=2.41 min. m/z=251 (M+1).

Some of the above compound (15 g, 59.9 mmol) was dissolved in toluene (100 ml), and ethylene glycol (18.6 g, 300 mmol) and p-toluene sulphonic acid (516 mg, 3 mmol) were added. The mixture was refluxed under nitrogen using a Dean-Stark water trap. After 3 h the solution had become a dark purple color, the reaction was cooled and TEA (2 ml) and water (100 ml) were added. The phases were separated, and the organic phase was washed with sat. NaHCO₃ (50 ml), dried over MgSO₄, and concentrated to yield a yellow oil (15.5 g). Ethylene glycol (3.7 ml; 60 mmol) was refluxed in toluene under nitrogen using a Dean-Stark water trap for 1 h. p-Toluene sulphonic acid (1.5 g) and the yellow oil (15.5 g) were added and the solution was refluxed for 3 h. The solution was cooled and pyridine (5 ml) was added. The solution was washed with sat. NaHCO₃ and 10% AcOH/water (100 ml each), dried over MgSO₄, and concentrated under vacuum to yield a yellowish orange oil (10.1 g). The crude product was dissolved in THF (30 ml) and a 1 M tetrabutylammonium fluoride solution in THF (41 ml; 41 mmol) was added. The mixture was stirred under nitrogen at room temperature for 30 min. Diethylether (300 ml) was added and the solution was washed with water (2×100 ml) and sat. NaCl (100 ml), dried over MgSO₄, and concentrated under vacuum to yield a brown oil (6.4 g). The compound was purified by flash chromatography (silica, 40 mm i.d.×15 cm, 1:2 AcOEt/heptane). The appropriate fractions were pooled and concentrated to yield a white crystalline solid (3.62 g). ¹H-NMR (300 MHz; CDCl₃) δ 1.65 ppm (s, 3H); 3.79 (m, 2H); 4.04 (m, 2H); 5.12 (br, 1H), 6.79 (d, 2H); 7.33 (d, 2H). LCMS (Method A): Rt=0.95 min. m/z=181 (M+1).

### Example (41)

### 6-O-[4-(2-Methyl-[1,3]dioxolan-2-yl)-phenyl]-L-galactono-1,4-lactone

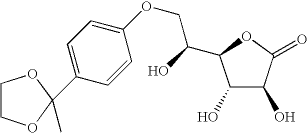

6-Bromo-6-deoxy-L-galactono-1,4-lactone can be prepared by the methods described in Chaveriat, L.; Stasik, I.; Demailly, G.; and Beaupère, D. Tetrahedron 60, 2079-2081 (2004), by employing the L-isomers as starting materials in place of the D-isomers or by saponification of 2,3,5-Tri-O-acetyl-6-bromo-6-deoxy-L-galactono-1,4-lactone. The 6-Bromo-6-deoxy-L-galactono-1,4-lactone can then be converted to the title compound by treating it with 4-(2-Methyl-1,3-dioxolan-2-yl)phenol and an appropriate base (e.g. K₂CO₃) in a suitable solvent (e.g. acetonitrile) at a temperature which allows the conversion to take place in a reasonable amount of time (e.g. refluxing acetonitrile). Standard work up procedures like extraction and flash chromatography can be used to isolate the product.

### Example (42)

### 6-O-[4-(2-Methyl-[1,3]dioxolan-2-yl)-phenyl]-β-L-galactopyranose

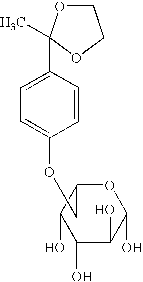

6-O-[4-(2-Methyl-[1,3]dioxolan-2-yl)-phenyl]-L-galactono-1,4-lactone can be converted to the title compound using methods analogous to those described in Binch, H.; Stangier, H. and Thiem, J. Carbohydrate Research 306, 409-419, (1998) (e.g. peracetylation with acetic anhydride, selective reduction with disiamylborane, and saponification with sodium methoxide).

### Example (43)

### 6-O-(4-acetylphenyl)-β-L-galactopyranose

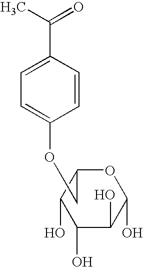

The deprotection of 6-O-[4-(2-Methyl-[1,3]dioxolan-2-yl)-phenyl]-β-L-galactopyranose can be facilitated by TFA or by one of the methods described in Greene, T. W. and Wuts, P. G. M. Protective Groups in Organic Synthesis, John Wiley and Sons, New York, 3^{rd} ed. (1999).

### Example (44)

### Guanosine-5′-(6-O-(4-acetylphenyl)-β-L-galactopyranosyl)-diphosphate dilithium salt

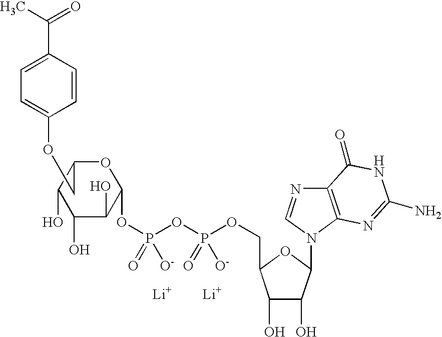

The title compound can be prepared from 6-O-(4-acetylphenyl)-β-L-galactopyranose using methods analogous to those found in Binch, H.; Stangier, H. and Thiem, J. Carbohydrate Research 306, 409-419, (1998).

### General Conjugation Procedure

### General Conditions

The pH of the buffers should be adjusted to such that the glycosyltransferase catalyses the reaction at a reasonable rate, while avoiding pH ranges which are not compatible with the protein which is to be modified. Examples of buffer types and pH ranges can be found in the literature (e.g. US Patent 20040063911A1, Gabenhorst, E.; Nimtz, M.; Costa, J. and Conradt, H. S. J. Biol. Chem. 273(47), 30985-30994 (1998), Uchiyama and Hindsgaul J. Carbohydr. Chem. 17, 1181 (1998), Stults, C. L. M. et al. Glycobiology 9(7), 661-668 (1999)).
The temperature should be adjusted to such that the glycosyltransferase catalyses the reaction at a reasonable rate, while avoiding temperature ranges which are not compatible with the protein which is to be modified. For example, temperatures which are too high can change the structure of some proteins (e.g. heat denaturation), thus leading to lower activities for enzymes, or lower receptor affinities for agonists and antagonists, or reduced biological function in general.

The protein or peptide to be modified is in a solution with an appropriate buffer. If the protein or peptide does not contain the desired functional group which is recognized by the transferase, it may need to be treated with the appropriate conditions as to insert or unmask this functional group (e.g. a complex N-glycan could be treated with neuraminidase and galactosidase in order to allow a galactosyl transferase to recognize the GlcNAc-acceptor motif). A representative procedure for enzymatic preparation of asialo agalacto glycoproteins is described in Haginaka 3; Matsunaga H, Chirality, 11 (1999), 426-431. Sialic acids may also be removed chemically as described in Kono M et al., Biochemical and Biophysical Research Communications Vol. 272, No. 1 pp. 94-97 (2000).

A solution of the donor substance (B-L-(O—PO₂)ₙ-A), e.g. a UDP, GDP or CMP-sugar) in an appropriate buffer is added to a solution of the protein (M′) in an appropriate buffer. A solution of a suitable transferase in an appropriate buffer is added. The addition of other chemicals may be added to facilitate the reaction (e.g. alkaline phosphatase can be added to degrade components which compete for the transferases active site, and α-lactalbumin may be added to reactions which use bovine β-1,4-galactosyl transferase, such that more diverse donor substrates are tolerated as described by Do, K; Do, S. and Cummings, R. D. J. Biol. Chem. 270(31), 18447-18451 (1995)). The amounts of transferase and donor substance per amount of protein to be modified may be experimentally determined to yield a reasonable amount of product in an appropriate amount of time. Some similar chemistries from which general conditions can be found are reported in the literature (e.g. US Patent 20040063911, Gabenhorst, E.; Nimtz, M.; Costa, J. and Conradt, H. S. J. Biol. Chem. 273(47), 30985-30994 (1998), Uchiyama and Hindsgaul J. Carbohydr. Chem. 17, 1181 (1998), Stults, C. L. M. et al. Glycobiology 9(7), 661-668 (1999)).

It may or may not be advantageous to purify the intermediate product (B-L-M or L′-M) before performing the next modification. Such a purification could be a complete purification (isolating only the product) using state of the art techniques which are generally known for the protein in question or a partial purification to remove components which might interfere with the next reaction (e.g. an ultrafiltration or dialysis to remove excess donor substance).

The intermediate product (B-L-M or L′-M) may then be mixed together with a modifying reactant (P′) to form the desired molecule (P—B′-L-M or P-L-M). The appropriate buffer, temperature and reaction times may be experimentally determined. Conditions or reagents which facilitate the reaction between B-L-M or L′-M and P′ would need to be used, and are known to those skilled in the art. In some cases the intermediate product (B-L-M or L′-M) may react with several molecules of the modifying reactant (P′). If a lower degree of modification is desired, fewer equivalents of the modifying reactant (P′) can be added or shorter reaction times or lower temperatures may be used. The final product can then be purified by state of the art methods.

### General Procedure for Coupling of Azide to Alkyne:

The reaction is known (see for example Kolb H C; Finn M G; Sharpless K B, Angewandte Chemie-International Edition 40(11), 2001, 2004-2021, and Chittaboina S; Xie F; Wang Q, Tetrahedron Letters, 46(13), 2005, 2331-2336) and is generally performed by mixing an alkyne containing compound with an azide containing molecule, optionally together with one or more catalyst in a suitable solvent. One of the components may be in excess in order to rapidly drive the reaction to completion. The reaction speed depends on concentrations, temperature and steric factors but is normally completed within 24 hours. The reaction is performed between 0-100° C., preferable between 20-40° C. Solvent or solvent mixtures are ideally chosen, so that they dissolve, or partially dissolve all reactants. The preferable catalyst is Cu(I) cations which may be generated in a number of ways, for example from a mixture of copper sulfate and sodium ascorbate as described in Chittaboina S; Xie F; Wang Q, Tetrahedron Letters, 46(13), 2005, 2331-2336. Water (optionally buffered) may be an ideal choice for polypeptides such as proteins or peptides. Polar co-solvents such as dimethylformamide, dimethyl sulfoxide, dioxane etc. may be added in order to dissolve one of both reaction components. Formation of the triazole addition product may be monitored by any standard technique appropriate for the given protein or peptide in question. Products are isolated using techniques suitable for the given polypeptide, for example using reverse or normal phase HPLC, ion-exchange chromatography, gel filtration techniques, affinity chromatography etc.

### General Procedure for Hydroxylamine Coupling to Aldehyde or Ketones:

The reaction is known (see for example Rose, J. Am. Chem. Soc. 1994, 116, 30-33 and European Patent 0243929) and is generally performed by mixing the aminooxy component and the aldehyde/ketone component in approximately equal molar proportions at a concentration of 1-10 mM in aqueous solution at mildly acid pH (2 to 6) at room temperature and the conjugation reaction (in this case oximation) followed by reversed phase high pressure liquid chromatography (HPLC) and electrospray ionisation mass spectrometry (ES-MS). The reaction speed depends on concentrations, pH and steric factors but is normally at equilibrium within a few hours, and the equilibrium is greatly in favour of conjugate (Rose, et al., Biacanjugate Chemistry 1996, 7, 552-556). A slight excess (up to five fold) of one component forces the conjugation reaction towards completion. Products are isolated using techniques suitable for the given polypeptide, for example using reverse or normal phase HPLC, ion-exchange chromatography, gel filtration techniques, affinity chromatography etc.

In the procedure described above, the reactive hydroxylamine group may be replaced by other reactive groups that can react with an aldehyde or a ketone, for example hydrazides, hydrazines, hydrazine carboxylates, semicarbazides, thiosemicarbazides, carbonic acid dihydrazide derivatives, carbazide derivatives, thiocarbazides and amines.

### General Procedure for Reacting Thiols with Electrophiles:

The reaction is known (J. Kubler-Kielb and V. Pozsgay, J. Org. Chem.; 70(17), 2005, 6987-6990) and is generally performed by mixing the thio component with the electrophile component (e.g. a α-haloacetamide, a α-haloketone or a α-haloester) in approximately equal molar amount, in an appropriate solvent, such as water, preferably buffered in order to minimize changes in pH during the reaction. One of the reaction components may be added in excess in order to rapidly drive the reaction to completion. Polar co-solvents such as dimethylformamide, dimethyl sulfoxide, dioxane etc. may be added in order to dissolve one of both reaction components. Product formation may be monitored by any standard technique appropriate for the given protein or peptide in question for example reversed phase high pressure liquid chromatography (HPLC) and electrospray ionisation mass spectrometry (ES-MS). Products are isolated using techniques suitable for the given polypeptide, for example using reverse or normal phase HPLC, ion-exchange chromatography, gel filtration techniques, affinity chromatography etc.

In the procedure described above, the reactive α-haloacetamide group, the reactive α-haloketone group or the reactive α-haloester group may be replaced by other reactive groups that can react with thiol, for example maleimides and alkyl halides, pyridyl disulfides and dialkyl disulfides.

### PEG Reagents

The PEG reagents used below are either commercially available or can be prepared via methods analogous to those described in the patent literature (WO2005035553, WO2005035565, WO2005035727). Branched PEG starting materials are available from NOF Corporation. PEG-CH═CH₂ may be prepare by mixing vinylacetic acid together with DIEA, EDAC and HOBt for a period of time in an appropriate solvent (e.g. 30 min. in DCM). The mixture is then added to a solution of m-PEG-NH₂ in an appropriate solvent (e.g. DCM). After mixing for an appropriate period of time (e.g. 16 h), the product can be isolated by precipitation (e.g. by adding diethyl ether). The precipitate can then be filtered off and washed with an appropriate solvent (e.g. diethylether), and dried under vacuum to yield the PEG reagent.

### Example 45

### Asialo FVIIa

FVIIa (30 ml, 1.39 mg/ml in 10 mM gly-gly, 10 mM CaCl₂, 50 mM NaCl, pH 6.0) was thawed and poured into a 50 ml centrifuge tube, and neuraminidase (2 units, 130 μl, sigma N-6514) was added. The mixture was allowed to stand at room temperature for 16 h. The sample was cooled on ice and 3.5 ml 100 mM EDTA-4Na and 100 μl 1 N NaOH was added. The pH was 8.18. This was purified using three serial connected 5 ml Hitrap Q columns and the following buffers on an Äkta Purifier with a flow of 1 ml/min.

Buffer A=25 mM MES, 50 mM NaCl, pH 8

Buffer B=25 mM MES, 50 mM NaCl, 25 mM CaCl₂, pH 6.

The solution was loaded onto the columns and buffer A (30 ml) was eluted through the columns. Buffer B was then eluted through the column, and the fractions containing asialo-FVIIa were collected and pooled to yield the product (14 ml, 2.4 mg/ml). (Note: The product can also be eluted with other buffers, e.g. Tris or Gly-gly).

### Asialo-Agalacto FVIIa

The terminal galactoses can be removed from asialo FVIIa by treating asialo FVIIa with a galactosidase in an appropriate buffer at a temperature and pH which allow for reaction completion in a reasonable amount of time, while maintaining reasonable biological activity for the product. Purification can be achieved in similar fashion to that described for asialo FVIIa.

### Example 46

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa in example 45. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | Modifying reactant (P′) |
|---|---|---|---|
| | Protein | Type of | and |
| Donor substance or salt thereof (B-L-(O—PO₂)ₙ-A) | (M′) | Transferase | (Special conditions) |
| 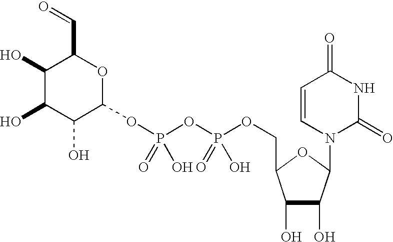 | AsialoagalactoFVIIa | galactosyl-transferase | PEG(20k)-ONH₂ |

### Example 47

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O-PO₂)ₙ-A) | (M′) | Transferase | reactant (P′) |
| 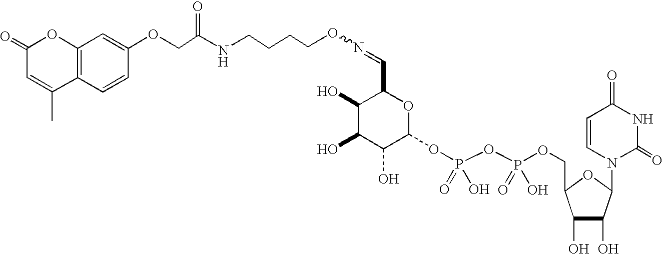 | AsialoagalactoFVIIa | galactosyl-transferase | PEG(20k)-CH═CH2(UV light) |

### Example 48

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O-PO₂)ₙ-A) | (M′) | Transferase | reactant (P′) |
| 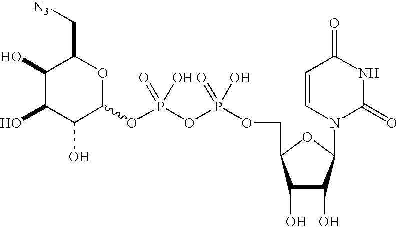 | AsialoagalactoFVIIa | galactosyl-transferase | 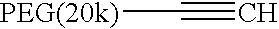 |

### Example 49

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 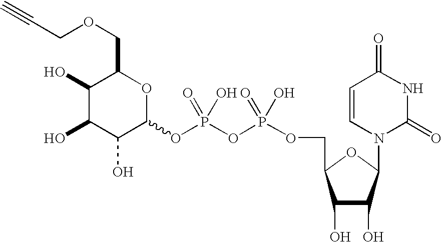 | AsialoagalactoFVIIa | galactosyl-transferase | PEG(20k)-N₃ |

### Example 50

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 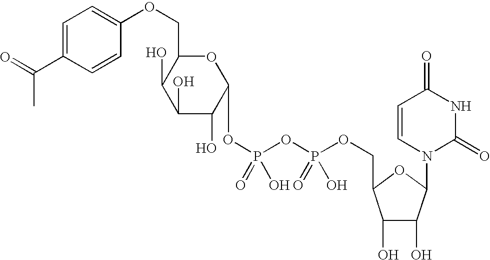 | AsialoagalactoFVIIa | galactosyl-transferase | PEG(20k)-ONH₂ |

### Example 51

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 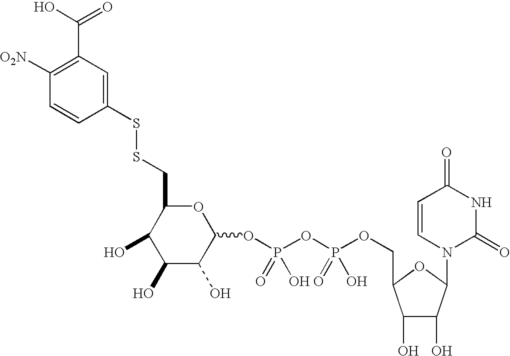 | AsialoagalactoFVIIa | galactosyl-transferase | PEG(20k)-SH |

### Example 52

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 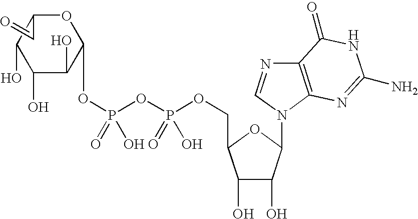 | FVIIa | fucosyltransferase | PEG(20k)-ONH₂ |

### Example 53

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 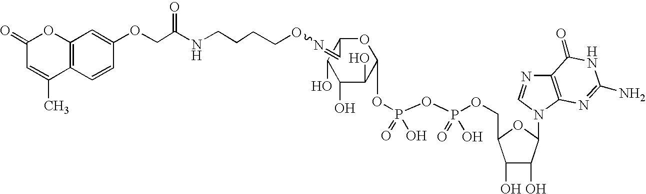 | FVIIa | fucosyltransferase | PEG(20k)-CH═CH2(UV light) |

### Example 54

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 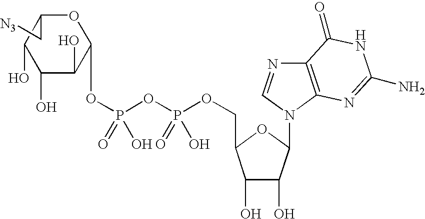 | FVIIa | fucosyltransferase | 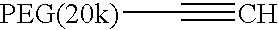 |

### Example 55

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 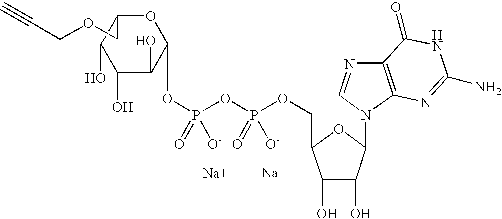 | FVIIa | fucosyltransferase | PEG(20k)-N₃ |

### Example 56

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 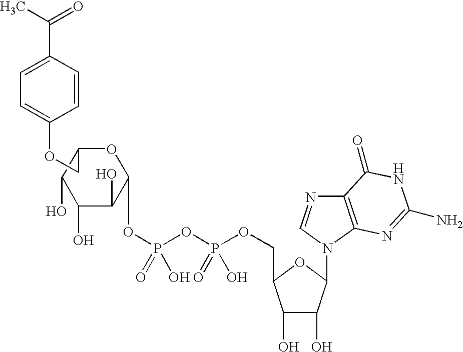 | FVIIa | fucosyltransferase | PEG(20k)-ONH₂ |

### Example 57

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 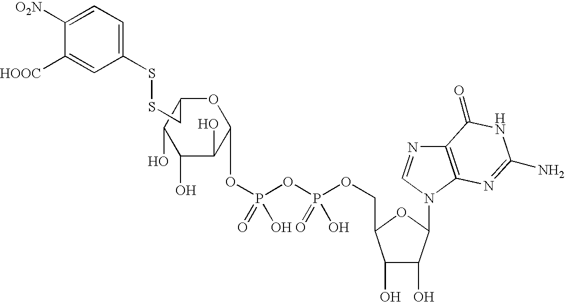 | FVIIa | fucosyltransferase | PEG(20k)-SH |

### Example 58

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | Modifying reactant (P′) |
|---|---|---|---|
| | Protein | Type of | and |
| Donor substance or salt thereof (B-L-(O—PO₂)ₙ-A) | (M′) | Transferase | (Special conditions) |
| 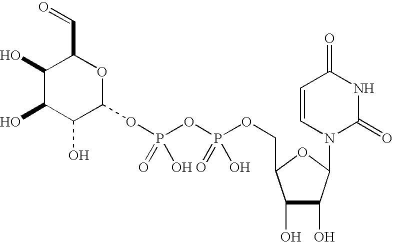 | AsialoagalactoFVIIa | galactosyl-transferase | PEG(10k)-ONH₂ |

### Example 59

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 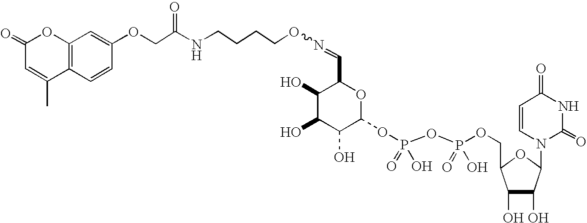 | AsialoagalactoFVIIa | galactosyl-transferase | PEG(10k)-CH═CH2(UV light) |

### Example 60

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 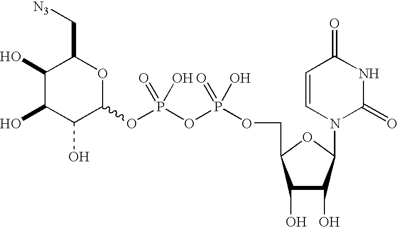 | AsialoagalactoFVIIa | galactosyl-transferase | 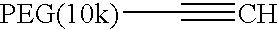 |

### Example 61

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 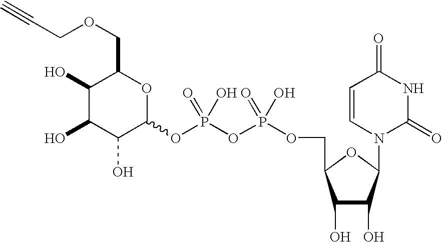 | AsialoagalactoFVIIa | galactosyl-transferase | PEG(10k)-N₃ |

### Example 62

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 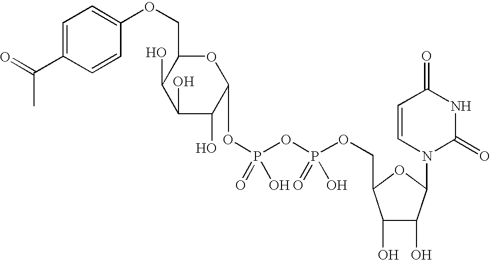 | AsialoagalactoFVIIa | galactosyl-transferase | PEG(10k)-ONH₂ |

### Example 63

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 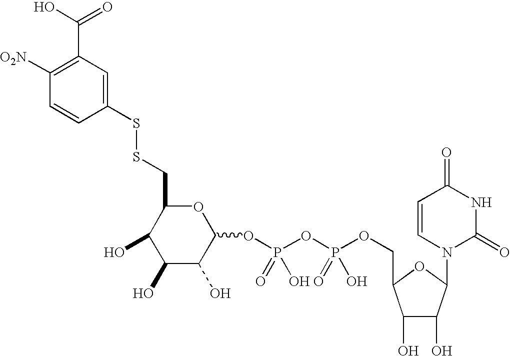 | AsialoagalactoFVIIa | galactosyl-transferase | PEG(10k)-SH |

### Example 64

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 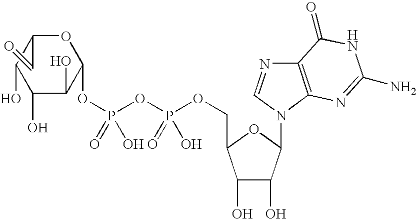 | FVIIa | fucosyltransferase | PEG(10k)-ONH₂ |

### Example 65

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

Donor substance or salt thereof (B-L-(O—PO₂)ₙ-A)

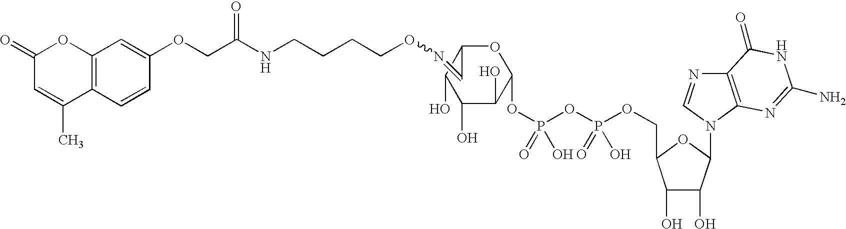

| Starting Protein (M′) | Type of Transferase | Modifying reactant (P′) |
|---|---|---|
| FVIIa | fucosyltransferase | PEG(10k)-CH═CH2 (UV light) |

### Example 66

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying reactant |
| Donor substance or salt thereof (B-L-(O—PO₂)ₙ-A) | (M′) | Transferase | (P′) |
| 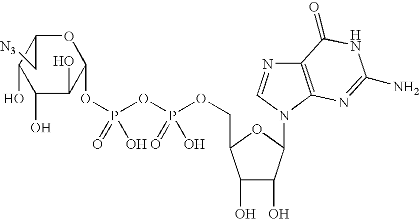 | FVIIa | fucosyltransferase | 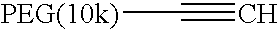 |

### Example 67

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 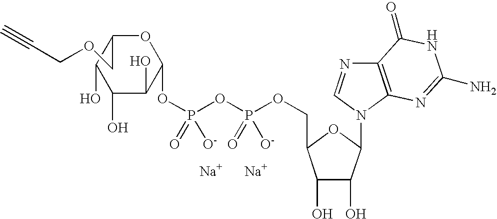 | FVIIa | fucosyltransferase | PEG(10k)-N₃ |

### Example 68

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 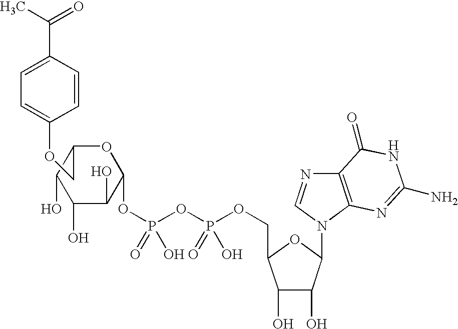 | FVIIa | fucosyltransferase | PEG(10k)-ONH₂ |

### Example 69

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | Modifying |
|---|---|---|---|
| | Protein | Type of | reactant |
| Donor substance or salt thereof (B-L-(O—PO₂)ₙ-A) | (M′) | Transferase | (P′) |
| 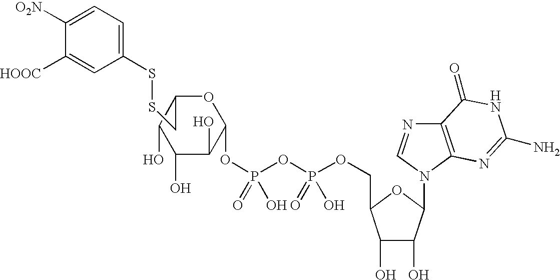 | FVIIa | fucosyltransferase | PEG(10k)-SH |

### Example 70

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | Modifying reactant (P′) |
|---|---|---|---|
| | Protein | Type of | and |
| Donor substance or salt thereof (B-L-(O—PO₂)ₙ-A) | (M′) | Transferase | (Special conditions) |
| 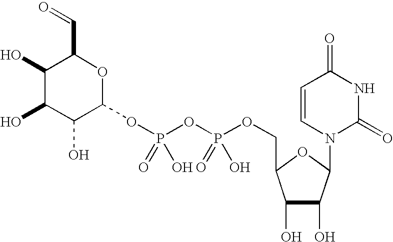 | AsialoagalactoFVIIa | galactosyl-transferase | PEG(10k)₂-ONH₂ |

### Example 71

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

Donor substance or salt thereof (B-L-(O—PO₂)ₙ-A)

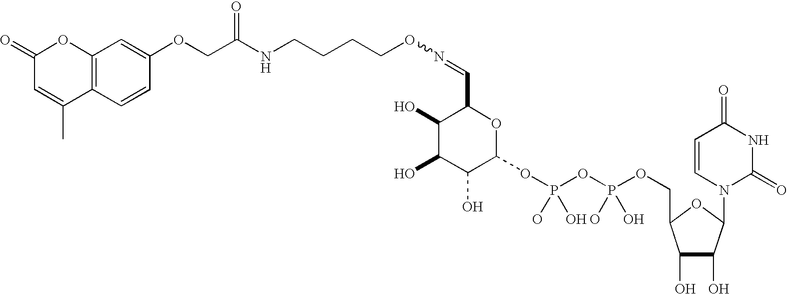

| Starting Protein (M′) | Type of Transferase | Modifying reactant (P′) |
|---|---|---|
| Asialo agalacto FVIIa | galactosyl-transferase | PEG(10k)₂-CH═CH2 (UV light) |

### Example 72

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | |
| Donor substance or salt thereof (B-L-(O—PO₂)ₙ-A) | (M′) | Transferase | Modifying reactant (P′) |
| 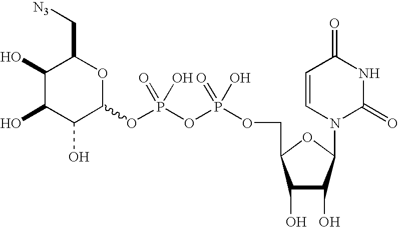 | AsialoagalactoFVIIa | galactosyl-transferase | 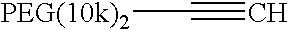 |

### Example 73

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying reactant |
| Donor substance or salt thereof (B-L-(O—PO₂)ₙ-A) | (M′) | Transferase | (P′) |
| 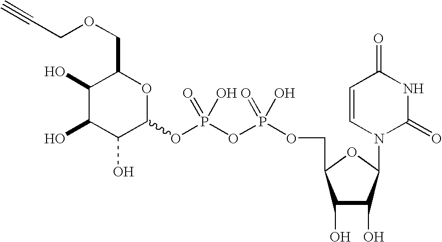 | AsialoagalactoFVIIa | galactosyl-transferase | PEG(10k)₂-N₃ |

### Example 74

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying reactant |
| Donor substance or salt thereof (B-L-(O—PO₂)ₙ-A) | (M′) | Transferase | (P′) |
| 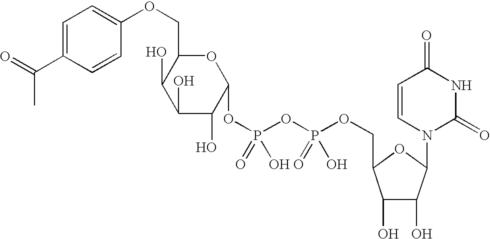 | AsialoagalactoFVIIa | galactosyl-transferase | PEG(10k)₂-ONH₂ |

### Example 75

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 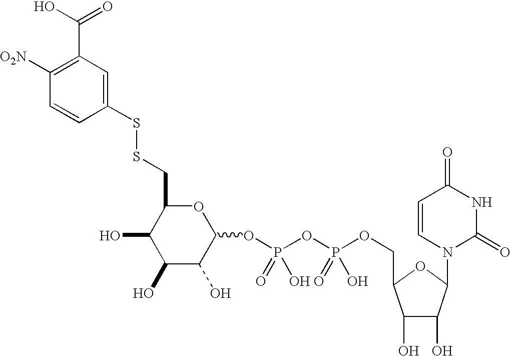 | AsialoagalactoFVIIa | galactosyl-transferase | PEG(10k)₂-SH |

### Example 76

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 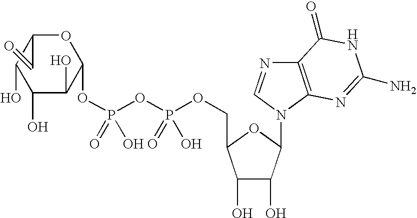 | FVIIa | fucosyltransferase | PEG(10k)₂-ONH₂ |

### Example 77

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

Donor substance or salt thereof (B-L-(O—PO₂)ₙ-A)

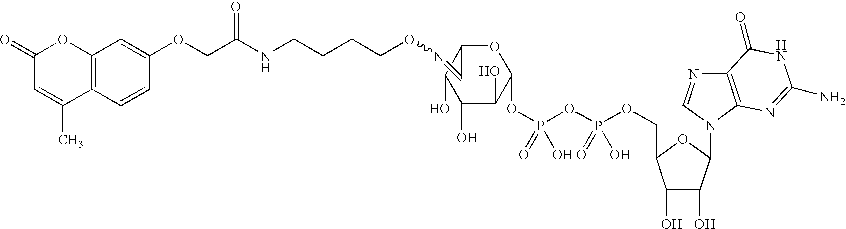

| Starting Protein (M′) | Type of Transferase | Modifying reactant (P′) |
|---|---|---|
| FVIIa | fucosyltransferase | PEG(10k)₂-CH═CH2 (UV light) |

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying reactant |
| Donor substance or salt thereof (B-L-(O—PO₂)ₙ-A) | (M′) | Transferase | (P′) |
| 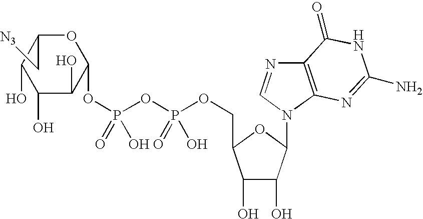 | FVIIa | fucosyltransferase | 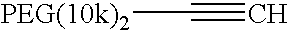 |

### Example 79

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 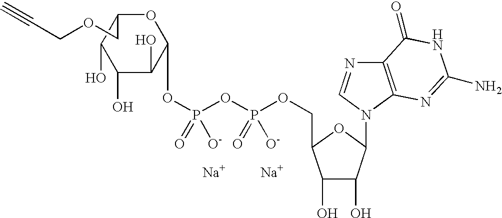 | FVIIa | fucosyltransferase | PEG(10k)₂-N₃ |

### Example 80

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 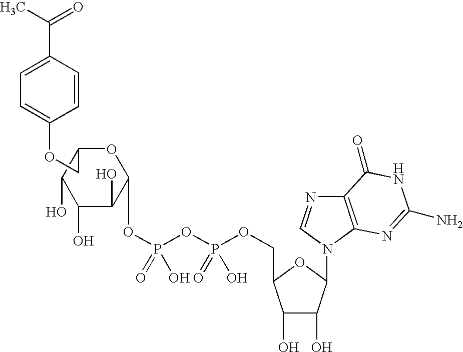 | FVIIa | fucosyltransferase | PEG(10k)₂-ONH₂ |

### Example 81

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 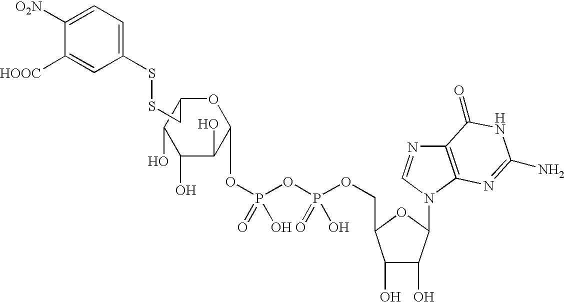 | FVIIa | fucosyltransferase | PEG(10k)₂-SH |

### Example 82

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | Modifying reactant (P′) |
|---|---|---|---|
| | Protein | Type of | and |
| Donor substance or salt thereof (B-L-(O—PO₂)ₙ-A) | (M′) | Transferase | (Special conditions) |
| 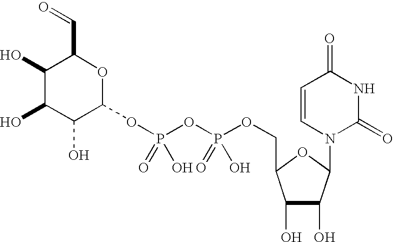 | AsialoagalactoFVIIa | galactosyl-transferase | PEG(20k)₂-ONH₂ |

### Example 83

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 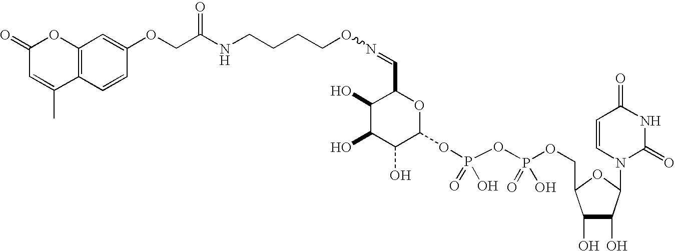 | AsialoagalactoFVIIa | galactosyl-transferase | PEG(20k)₂-CH═CH2(UV light) |

### Example 84

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 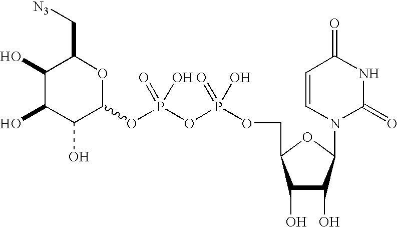 | AsialoagalactoFVIIa | galactosyl-transferase | 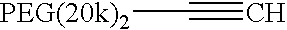 |

### Example 85

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 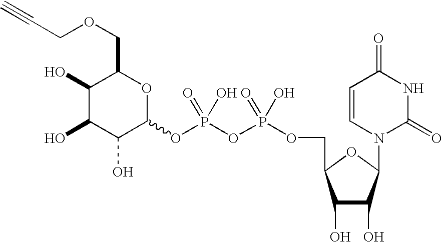 | AsialoagalactoFVIIa | galactosyl-transferase | PEG(20k)₂-N₃ |

### Example 86

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 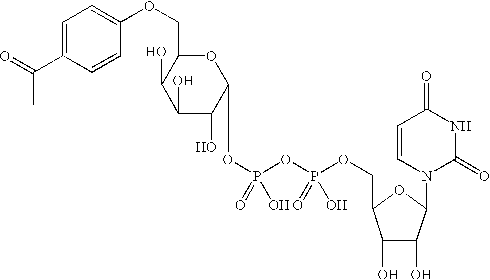 | AsialoagalactoFVIIa | galactosyl-transferase | PEG(20k)₂-ONH₂ |

### Example 87

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 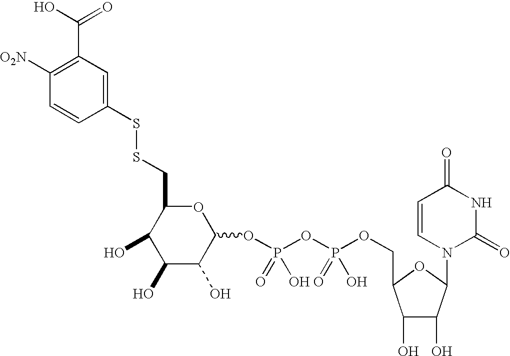 | AsialoagalactoFVIIa | galactosyl-transferase | PEG(20k)₂-SH |

### Example 88

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 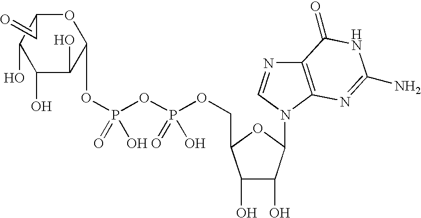 | FVIIa | fucosyltransferase | PEG(20k)₂-ONH₂ |

### Example 89

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 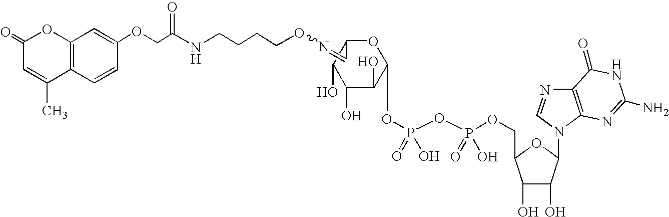 | FVIIa | fucosyl-transferase | PEG(20k)₂-CH═CH2(UV light) |

### Example 90

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 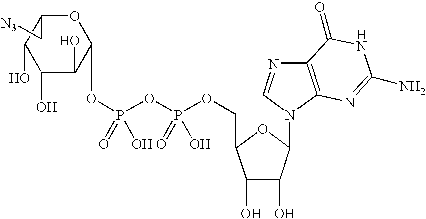 | FVIIa | fucosyltransferase | 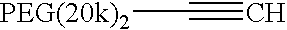 |

### Example 91

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 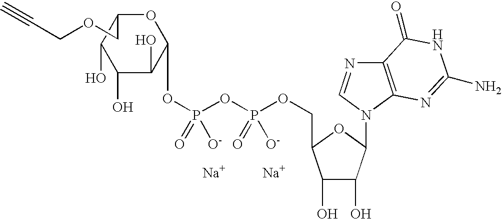 | FVIIa | fucosyltransferase | PEG(20k)₂-N₃ |

### Example 92

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 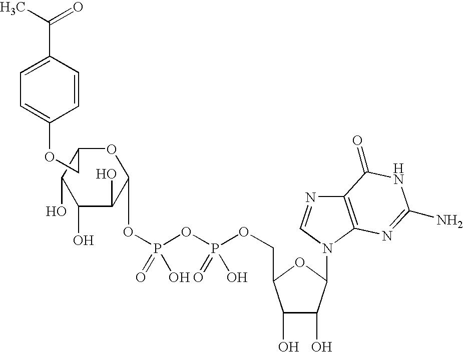 | FVIIa | fucosyltransferase | PEG(20k)₂-ONH₂ |

### Example 93

### Pegylation of FVIIa

Using the general conjugation procedure described above, the donor substance (B-L-(O—PO₂)ₙ-A), the starting protein (M′) and the transferase from the table below can be combined to form the intermediate product (B-L-M). The intermediate product can be purified by ion exchange chromatography in a similar manner as described for asialo FVIIa. The intermediate product (B-L-M) can be mixed with the modifying reactant (P′) according to the general conjugation procedure described above to form the desired pegylated FVIIa compound. The product can be purified by ion exchange chromatography in a similar manner as described for asialo FVIIa or by sized exclusion chromatography (e.g. using a High Load Superdex 200), or by a combination of the two methods.

| | Starting | | |
|---|---|---|---|
| | Protein | Type of | Modifying |
| Donor substance or salt thereof (B-L-(O—PO₂)ₙ-A) | (M′) | Transferase | reactant (P′) |
| 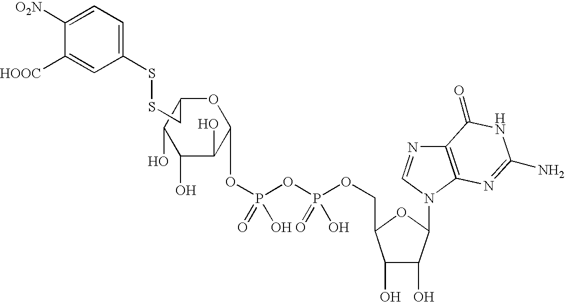 | FVIIa | fucosyltransferase | PEG(20k)₂-SH |

### EXEMPLARY EMBODIMENTS AND FEATURES OF THE INVENTION

To better illustrate the invention described herein, a nonlimiting list of some of exemplary embodiments and features of the invention is provided here:

1. A method for preparing a modified analogue P—B′-L-M of a starting molecule M′, where said modified analogue has improved pharmacologic properties compared to the starting molecule, the method comprising the consecutive steps of

a) reacting, in the presence of a glycosyltransferase, the starting molecule M′ comprising a reactive group, with a donor substance having the formula I

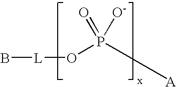

wherein

x=1 or 2,

A is selected from

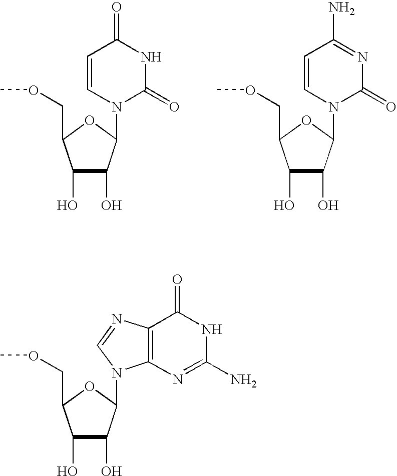

L is a divalent moiety, a bond, or a monovalent moiety L′, which comprises a protected or non-protected reactive group, which is not accessible in M′ and which specifically can react with other reactive groups, and

B is absent if L is L′ or B is a moiety which comprises a protected or non-protected reactive group, which is not accessible in M′ and which specifically can react with other reactive groups,

to yield an intermediary modified analogue of the starting molecule, said intermediary modified analogue having the formula B-L-M or L′-M, where M is M′, wherein the reactive group is absent or has been rendered substantially non-reactive,

b) if necessary, unprotecting the reactive group in B, and

c) conjugating said intermediary modified analogue to a molecule of formula P′ which comprises a reactive group not accessible in L and M and which specifically can react with B in said intermediate B-L-M to yield the modified analogue having formula P—B′-L-M, where P is P′ where the reactive group is absent or has been rendered substantially non-reactive, where B′ is a bond or B where the reactive group is absent or has been rendered substantially non-reactive, or when B is not present P′ can react with L′ in said intermediate L′-M to yield β-L-M, where L is L′ where the reactive group is absent or has been rendered substantially non-reactive.

2. The method according to embodiment 1, wherein the starting molecule is a glycosylated or a serine, threonine, lysine, asparagine, glutamine, tryptophane, tyrosine, cystine, arginine, histidine, glutamic acid, aspartic acid, hydroxyproline, gamma-carboxyglutamic acid containing polypeptide or protein.

3. The method according to embodiment 2, wherein the starting molecule is a glycosylated or a serine or threonine containing polypeptide or protein.

4. The method according to embodiment 2 or 3, wherein the polypeptide or protein is N-glycosylated or O-glycosylated.

5. The method according to any one of the preceding embodiments, wherein the reactive group in M′ is present in the glycosyl moiety.

6. The method according to any one of the preceding embodiments, wherein P is different from a biotinyl group.

7. The method according to any one of the preceding embodiments, which comprises the further step of confirming that the modified analogue has improved pharmacologic properties compared to the starting molecule.

8. The method according to any one of the preceding embodiments, wherein the improved pharmacologic property is selected from the group consisting of increased bioavailability, increased functional in vivo half-life, increased in vivo plasma half-life, reduced immunogenicity, increased protease resistance, increased affinity for albumin, improved affinity for a receptor, increased storage stability, decreased functional in vivo half-life, decreased in vivo plasma half-life.

9. The method according to embodiment 8, wherein the increased half-life is obtained by P being a group that increases molecular weight so that renal clearance is reduced or abolished and/or by P being a group that masks binding partners for hepatic receptors.

10. The method according to embodiment 8, wherein the reduced immunogenicity is obtained by P being a group which blocks antibody binding to immunogenic sites.

11. The method according to embodiment 8, wherein the improved affinity for albumin is obtained by P being a group which has high affinity for albumin.

12. The method according to embodiment 8, wherein the improved affinity for a receptor is obtained by P being a group which specifically binds a surface receptor on a target cell.

13. The method according to any one of the preceding embodiments, wherein P is selected from the group consisting of: a low molecular weight organic charged radical, which may contain one or more carboxylic acids, amines, sulfonic acids, phosphonic acids, or combinations thereof; a low molecular weight neutral hydrophilic molecule, such as cyclodextrin or a optionally branched polyethylene chain; a low molecular weight hydrophobic molecule such as a fatty acid or cholic acid or derivatives thereof; a polyethylene glycol with an average molecular weight of 2-40 kDa; a well-defined precision polymer such as a dendrimer with an exact molecular mass ranging from 700 Da to 20 kDa; a substantially non-immunogenic polypeptide such as albumin, an antibody or a part of an antibody optionally containing a Fc-domain; and a high molecular weight organic polymer.

14. The method according to any to any one of the preceding embodiments, wherein P is selected from the group consisting of a dendrimer, polyalkylene oxide (PAO), including polyalkylene glycol (PAG), such as polyethylene glycol (PEG) and polypropylene glycol (PPG), branched PEG, polyvinyl alcohol (PVA), polycarboxylate, poly-vinylpyrolidone, polyethylene-co-maleic acid anhydride, polystyrene-co-maleic acid anhydride, dextran, carboxymethyl-dextran.

15. The method according to any one of embodiments 1-13, wherein P is selected from the group consisting of a serum protein binding-ligand and a small organic molecule containing moieties that under physiological conditions alters charge properties, a structure which inhibits glycans from binding to receptors, and a neutral substituent that prevent glycan specific recognition.

16. The method according to any one of embodiments 1-15, wherein P′ comprises a functional group selected from the group consisting of any free amino, carboxyl, thiol, alkyl halide, acyl halide, chloroformiate, aryloxycarbonate, hydroxyl, α-haloacetamide, maleimide, azide, carbonyl group or aldehyde group; a carbonate such as p-nitrophenyl or succinimidyl; carbonyl imidazole; carbonyl chloride; carboxylic acid activated in situ; carbonyl halides; an activated ester such as an N-hydroxysuccinimide ester, an N-hydroxybenzotriazole ester, esters such as those comprising 1,2,3-benzotriazin-4(3H)-one; phosphoramidite; H-phosphonates; a phosphor triester or phosphor diester activated in situ; isocyanates; isothiocyanates; NH₂, OH, N₃, NHR′, OR′, O—NH₂, alkyne, alkene, diene, α,β-unsaturated ketone, α,β-unsaturated ester, α,β-unsaturated amide, 3-carboxy-4-nitrophenyldisulfanyl, pyridin-2-yldisulfany,

hydrazine derivatives —NH—NH₂,
hydrazine carboxylate derivatives —O—C(O)—NH—NH₂,
semicarbazide derivatives —NH—C(O)—NH—NH₂,
thiosemicarbazide derivatives —NH—C(S)—NH—NH₂,
carbonic acid dihydrazide derivatives —NHC(O)—NH—NH—C(O)—NH—NH₂,
carbazide derivatives —NH—NH—C(O)—NH—NH₂,
thiocarbazide derivatives —NH—NH—C(S)—NH—NH₂,
aryl hydrazine derivatives —NH—C(O)—C₆H₄—NH—NH₂,
hydrazide derivatives —C(O)—NH—NH₂; and
oxylamine derivatives, such as —C(O)—O—NH₂, —NH—C(O)—O—NH₂ and —NH—C(S)—O—NH₂.

17. The method according to any one of the preceding embodiments, wherein B comprises a functional group selected from the group consisting of any free amino, carboxyl, thiol, alkyl halide, acyl halide, chloroformiate, aryloxycarbonate, hydroxyl, α-haloacetamide, maleimide, azide, carbonyl group or aldehyde group; a carbonate such as p-nitrophenyl or succinimidyl; carbonyl imidazole; carbonyl chloride; carboxylic acid activated in situ; carbonyl halides; an activated ester such as an N-hydroxysuccinimide ester, an N-hydroxybenzotriazole ester, esters such as those comprising 1,2,3-benzotriazin-4(3H)-one; phosphoramidite; H-phosphonates; a phosphor triester or phosphor diester activated in situ; isocyanates; isothiocyanates; NH₂, OH, N₃, NHR′, OR′, O—NH₂, alkyne, alkene, diene, α,β-unsaturated ketone, α,β-unsaturated ester, α,β-unsaturated amide, 3-carboxy-4-nitrophenyldisulfanyl, pyridin-2-yldisulfany,

hydrazine derivatives —NH—NH₂,
hydrazine carboxylate derivatives —O—C(O)—NH—NH₂,
semicarbazide derivatives —NH—C(O)—NH—NH₂,
thiosemicarbazide derivatives —NH—C(S)—NH—NH₂,
carbonic acid dihydrazide derivatives —NHC(O)—NH—NH—C(O)—NH—NH₂,
carbazide derivatives —NH—NH—C(O)—NH—NH₂,
thiocarbazide derivatives —NH—NH—C(S)—NH—NH₂,
aryl hydrazine derivatives —NH—C(O)—C₆H₄—NH—NH₂,
hydrazide derivatives —C(O)—NH—NH₂; and
oxylamine derivatives, such as —C(O)—O—NH₂, —NH—C(O)—O—NH₂ and —NH—C(S)—O—NH₂.

18. The method according to any one of the preceding embodiments, wherein L and L′ are selected from the group consisting of a linear or branched divalent organic radical, a cyclic divalent organic radical, and a bond.

19. The method according to embodiment 18, wherein the linear divalent organic radical includes a multiply functionalized linear or branched alkyl group containing up to 18 carbon atoms.

20. The method according to embodiment 19, wherein the multiply functionalized alkyl group contains between 2 and 10 carbon atoms.

21. The method according to embodiment 19 or 20, wherein the alkyl chain(s) include(s) at least 1 atom different from carbon.

22. The method according to embodiment 21, wherein the at least 1 atom different from carbon is selected from the group consisting of N, O, and S.

23. The method according to embodiment 18, wherein L and L′ are a 5-7 membered ring.

24. The method according to embodiment 23, wherein the 5-7 membered ring structure contains at least one heteroatom independently selected from N, O or S.

25. The method according to any one of the preceding embodiments, wherein the donor substance has the general formula selected from

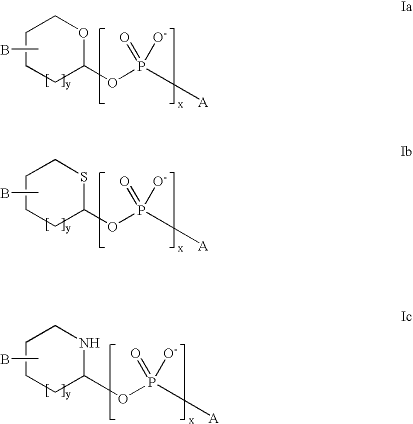

wherein y is 0, 1, or 2;

and optionally wherein any one carbon in the ring structure independently is substituted with hydroxy, hydroxymethyl, N-acylamino, alkyl, alkyloxy, halogen, alkanoyl, aryl, aryloxy, heteroaryl, or heteroaryloxy.

26. The method according to any one of the preceding embodiments, wherein the donor substance has the general formula Id

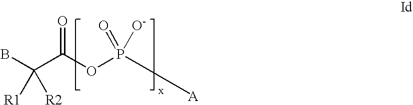

wherein R1 and R2 each independently are selected from hydrogen, alkyl, halogen, alkanoyl, aryl and heteroaryl.

27. The method according to embodiment 17, wherein L and L′ are selected from a group selected from

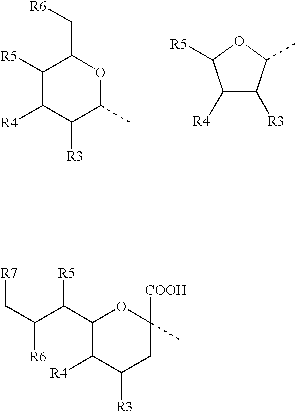

wherein one of R3-R7 is a divalent organic radical attached to B in general Formula I or a valency bond to B in general Formula I, and wherein the remaining R3-R7 each are selected independently from —H, —OH, —CH₂OH, —NH₂, N-acylamino groups including —NHAc, alkyl, alkyloxy, halogene, alkanoyl, aryl, aryloxy, heteroaryl and heteroaryloxy.

28. The method according to any one of the preceding embodiments, wherein B is absent, and wherein L′ is selected from the group consisting of

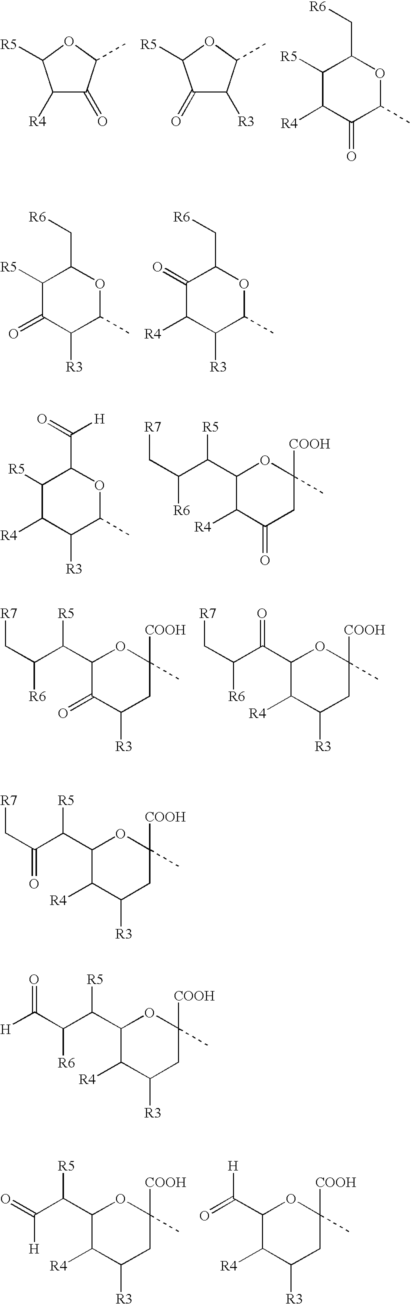

where R3-R7 independently are as defined in embodiment 27.

29. The method according to any one of the preceding embodiments, wherein the donor substance has the formula selected from the group consisting of

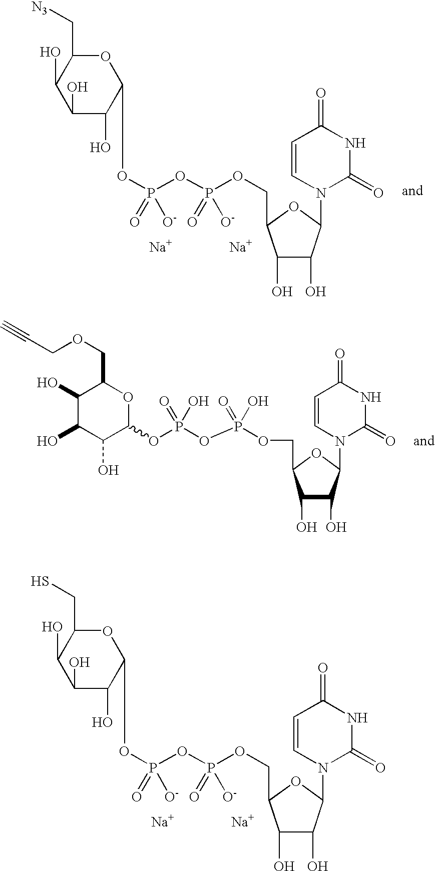

including a compound where the thiol group is protected as a mixed disulfide, and

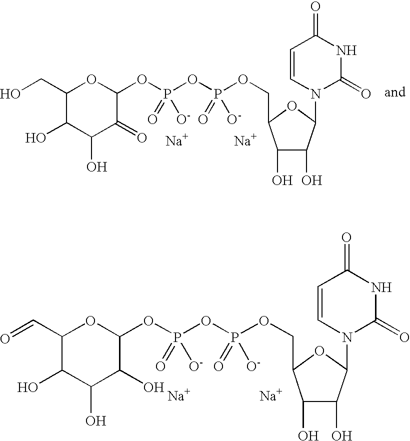

including any germinal diol forms thereof, and

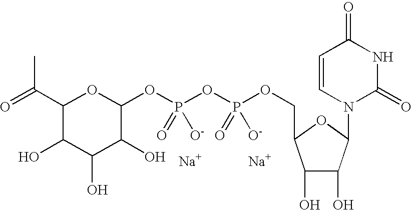

including any germinal diol forms thereof, and

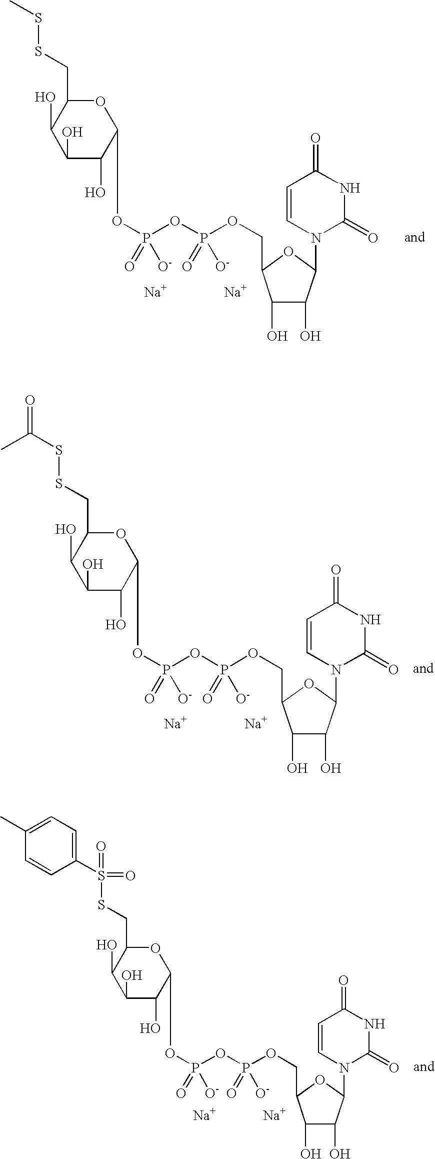

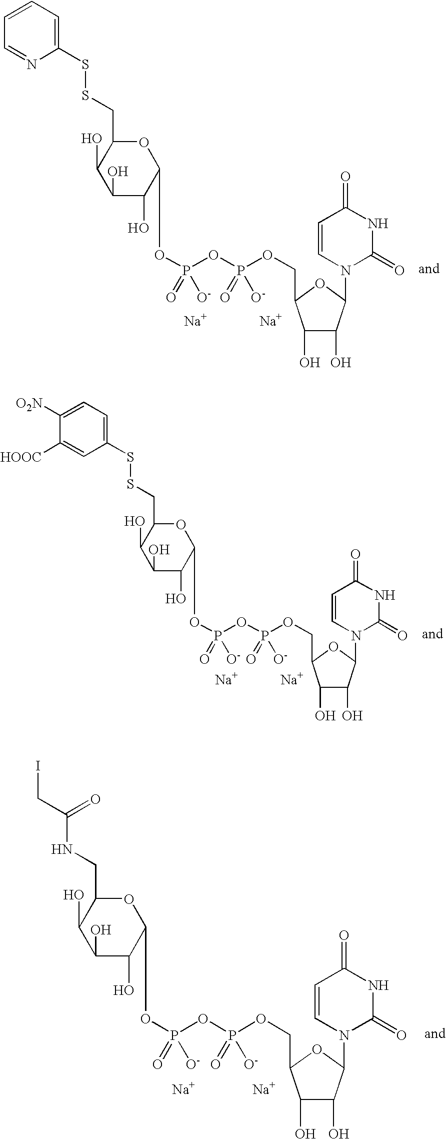

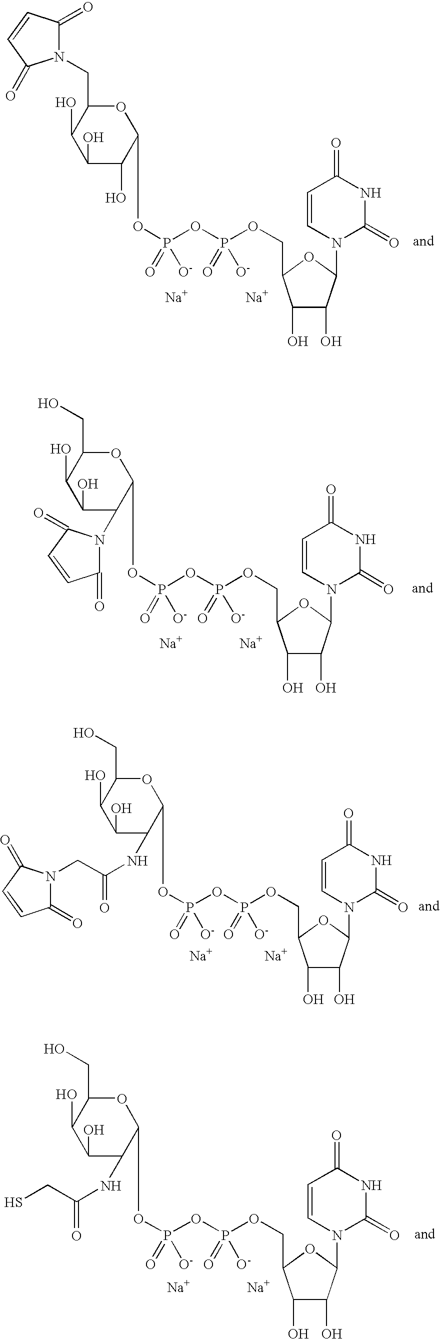

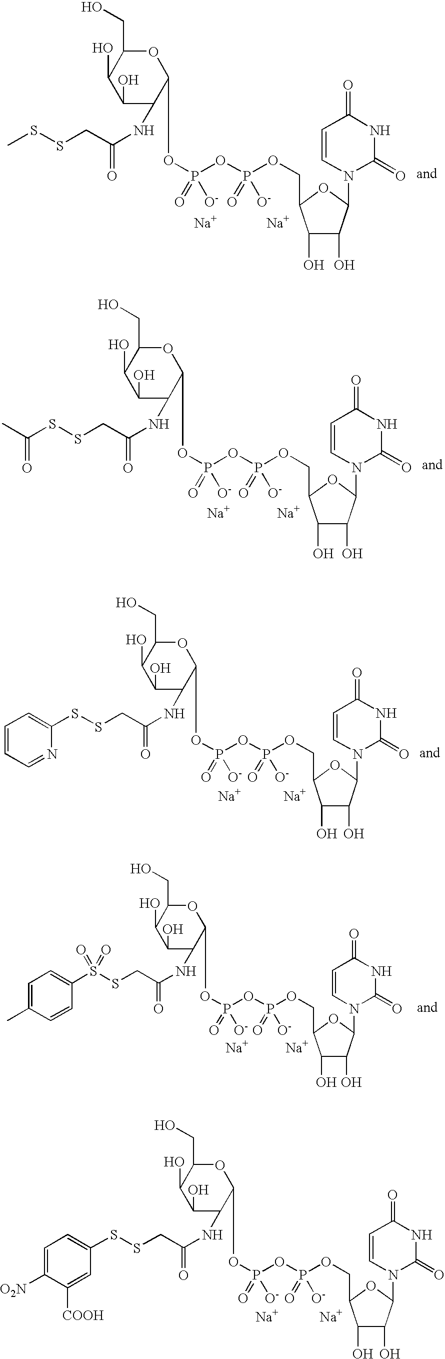

as well as any stereo isomers or other salts than sodium salts of the compounds selected from said group.

30. The method according to any one of the preceding embodiments, wherein M′ is selected from FVII, FVIII, FIX, FX, FII, FV, protein C, protein S, tPA, PAI-1, tissue factor, FXI, FXII, FXIII, as well as sequence variants thereof; immunoglobulins, cytokines such as interleukins, alpha-, beta-, and gamma-interferons, colony stimulating factors including granulocyte colony stimulating factors, platelet derived growth factors, phospholipase-activating protein (PUP), insulin, plant proteins such as lectins and ricins, tumor necrosis factors and related alleles, soluble forms of tumor necrosis factor receptors, interleukin receptors and soluble forms of interleukin receptors, growth factors such as tissue growth factors, such as TGFa's or TGFps and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and immunoglobulins such as IgG, IgE, IgM, IgA, and IgD, and fragments thereof, or any fusion proteins comprising any of the above mentioned proteins or fragments thereof.

31. A method for the preparation of a modified intermediate of formula B-L-M or L′-M as defined in any one of embodiments 1-5 and 15-29, said method comprising steps a and b but omitting step c of the method according to embodiments 1-5 and 15-30.

32. A donor substance having the general formula defined in any one of embodiments 1-5 and 15-30.

33. A modified intermediate of formula B-L-M or L′-M as defined in any one of embodiments 1-5 and 17-30.

34. A donor substance selected from the list consisting of

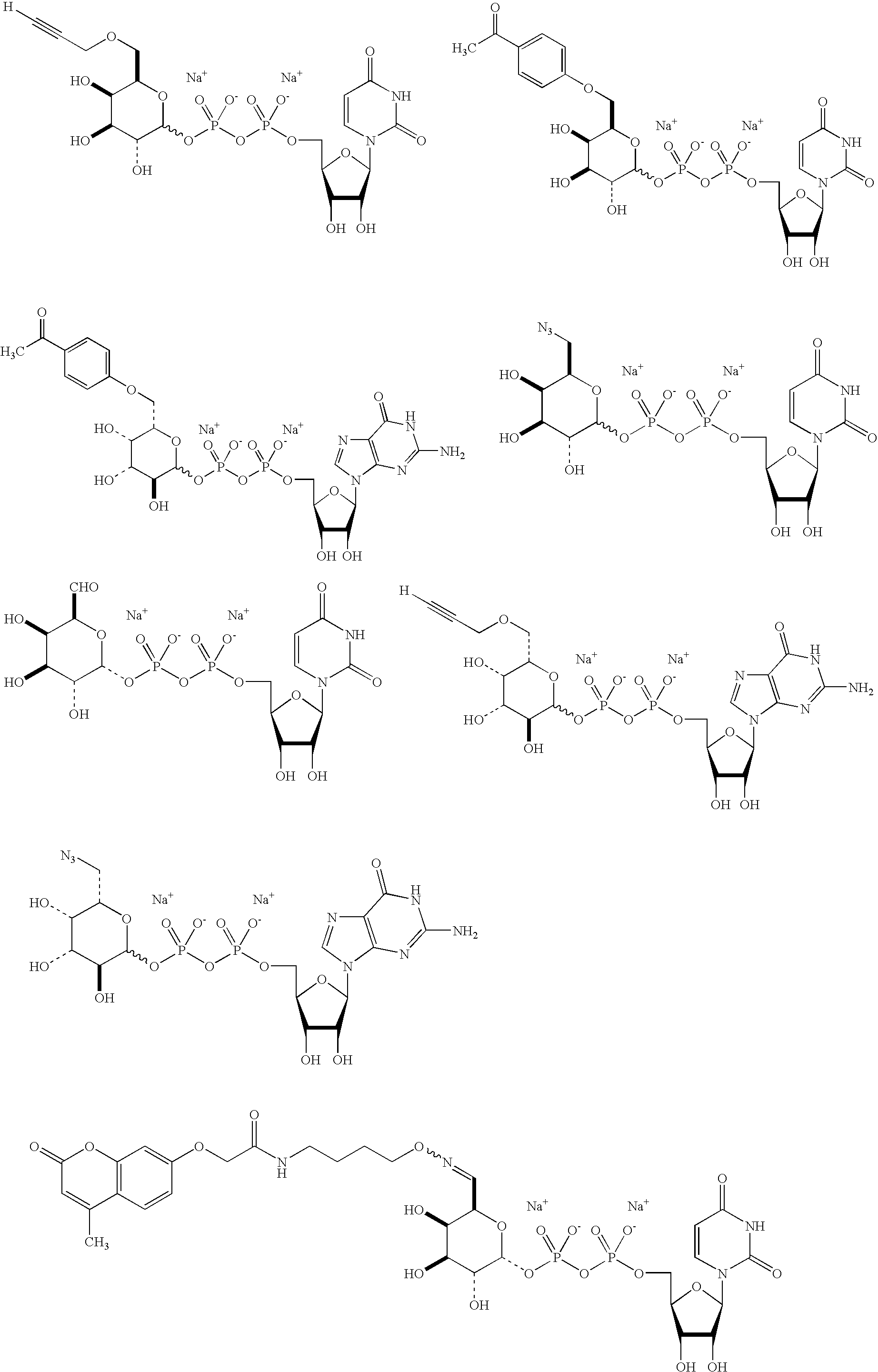

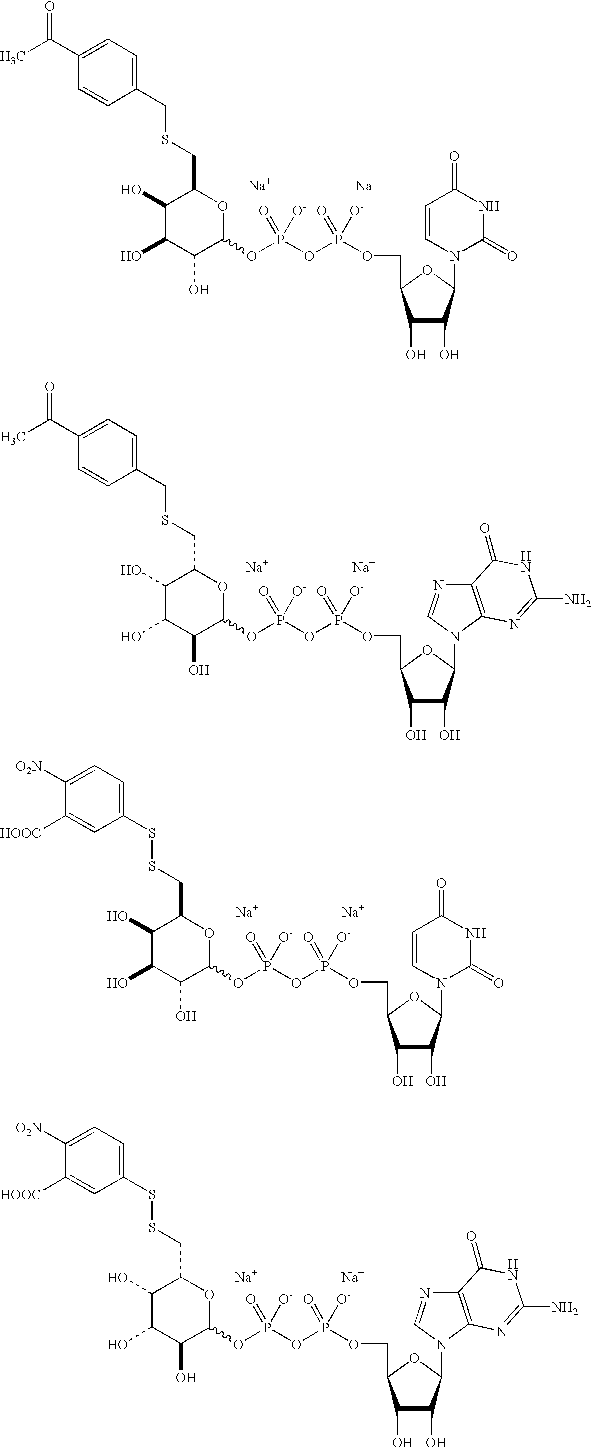

as well as any stereo isomers or other salts than sodium salts of these compounds.

35. A modified analogue P—B′-L-M or β-L-M, obtainable by the method according to any one of embodiments 1-30, wherein P, B′, L, and M are as defined in embodiment 1.

36. A pharmaceutical composition comprising a modified analogue P—B′-L-M or β-L-M according to embodiment 1-30, in a mixture with a pharmaceutically acceptable carrier, diluent, vehicle or excipient.

37. The modified analogue P—B′-L-M or β-L-M according to embodiment 35 for use in therapy.

1. A method for preparing a modified analogue P—B′-L-M of a starting molecule M′, where said modified analogue has improved pharmacologic properties compared to the starting molecule, the method comprising the consecutive steps of
a) reacting, in the presence of a glycosyltransferase, the starting molecule M′ comprising a reactive group, with a donor substance having the formula I 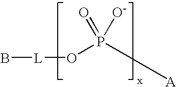
wherein
x=1 or 2,
A is selected from 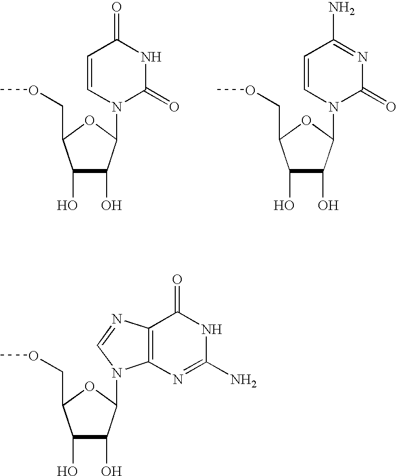
L is a divalent moiety, a bond, or a monovalent moiety L′, which comprises a protected or non-protected reactive group, which is not accessible in M′ and which specifically can react with other reactive groups, and
B is absent if L is L′ or B is a moiety which comprises a protected or non-protected reactive group, which is not accessible in M′ and which specifically can react with other reactive groups,
to yield an intermediary modified analogue of the starting molecule, said intermediary modified analogue having the formula B-L-M or L′-M, where M is M′, wherein the reactive group is absent or has been rendered substantially non-reactive,
b) if necessary, unprotecting the reactive group in B, and
c) conjugating said intermediary modified analogue to a molecule of formula P′ which comprises a reactive group not accessible in L and M and which specifically can react with B in said intermediate B-L-M to yield the modified analogue having formula P—B′-L-M, where P is P′ where the reactive group is absent or has been rendered substantially non-reactive, where B′ is a bond or B where the reactive group is absent or has been rendered substantially non-reactive, or when B is not present P′ can react with L′ in said intermediate L′-M to yield β-L-M, where L is L′ where the reactive group is absent or has been rendered substantially non-reactive.

2. The method according to claim 1, wherein the starting molecule is a glycosylated or a serine-containing, threonine-containing, lysine-containing, asparagine-containing, glutamine-containing, tryptophane-containing, tyrosine-containing, cystine-containing, arginine-containing, histidine-containing, glutamic acid-containing, aspartic acid-containing, or hydroxyproline-containing, gamma-carboxyglutamic acid-containing polypeptide or protein.

3. The method according to claim 2, wherein the starting molecule is a glycosylated or a serine-containing or threonine-containing polypeptide or protein.

4. The method according to claim 3, wherein the polypeptide or protein is N-glycosylated or O-glycosylated.

5. The method according to claim 1, wherein the reactive group in M′ is present in the glycosyl moiety.

6. The method according to claim 1, wherein P is different from a biotinyl group.

7. The method according to claim 1, which comprises the further step of confirming that the modified analogue has improved pharmacologic properties compared to the starting molecule.

8. The method according to claim 7, wherein the improved pharmacologic property is selected from the group consisting of increased bioavailability, increased functional in vivo half-life, increased in vivo plasma half-life, reduced immunogenicity, increased protease resistance, increased affinity for albumin, improved affinity for a receptor, increased storage stability, decreased functional in vivo half-life, and decreased in vivo plasma half-life.

9. The method according to claim 8, wherein the increased half-life is obtained by P being a group that increases molecular weight so that renal clearance is reduced or abolished and/or by P being a group that masks binding partners for hepatic receptors.

10. The method according to claim 8, wherein the reduced immunogenicity is obtained by P being a group which blocks antibody binding to immunogenic sites.

11. The method according to claim 8, wherein the improved affinity for albumin is obtained by P being a group which has high affinity for albumin.

12. The method according to claim 8, wherein the improved affinity for a receptor is obtained by P being a group which specifically binds a surface receptor on a target cell.

13. The method according to claim 1, wherein P is selected from the group consisting of: a low molecular weight organic charged radical, which may contain one or more carboxylic acids, amines, sulfonic acids, phosphonic acids, or combinations thereof; a low molecular weight neutral hydrophilic molecule, such as cyclodextrin or a optionally branched polyethylene chain; a low molecular weight hydrophobic molecule such as a fatty acid or cholic acid or derivatives thereof; a polyethylene glycol with an average molecular weight of 2-40 kDa; a well-defined precision polymer such as a dendrimer with an exact molecular mass ranging from 700 Da to 20 kDa; a substantially non-immunogenic polypeptide such as albumin, an antibody or a part of an antibody optionally containing a Fc-domain; and a high molecular weight organic polymer.

14. The method according to claim 1, wherein P is selected from the group consisting of a dendrimer, polyalkylene oxide (PAO), including polyalkylene glycol (PAG), such as polyethylene glycol (PEG) and polypropylene glycol (PPG), branched PEG, polyvinyl alcohol (PVA), polycarboxylate, poly-vinylpyrolidone, polyethylene-co-maleic acid anhydride, polystyrene-co-maleic acid anhydride, dextran, carboxymethyl-dextran.

15. The method according to claim 1, wherein P is selected from the group consisting of a serum protein binding-ligand and a small organic molecule containing moieties that under physiological conditions alters charge properties, a structure which inhibits glycans from binding to receptors, and a neutral substituent that prevent glycan specific recognition.

16. The method according to claim 1, wherein P′ comprises a functional group selected from the group consisting of any free amino, carboxyl, thiol, alkyl halide, acyl halide, chloroformiate, aryloxycarbonate, hydroxyl, α-haloacetamide, maleimide, azide, carbonyl group or aldehyde group; a carbonate such as p-nitrophenyl or succinimidyl; carbonyl imidazole; carbonyl chloride;
carboxylic acid activated in situ; carbonyl halides; an activated ester such as an N-hydroxysuccinimide ester, an N-hydroxybenzotriazole ester, esters such as those comprising 1,2,3-benzotriazin-4(3H)-one; phosphoramidite; H-phosphonates; a phosphor triester or phosphor diester activated in situ; isocyanates; isothiocyanates; NH₂, OH, N₃, O—NH₂, alkyne, alkene, diene, [symbol] β-unsaturated ketone, [symbol] β-unsaturated ester, [symbol] β-unsaturated amide, 3-carboxy-4-nitrophenyldisulfanyl, pyridin-2-yldisulfany, hydrazine derivatives, hydrazine carboxylate derivatives, semicarbazide derivatives, thiosemicarbazide derivatives, carbonic acid dihydrazide derivatives, carbazide derivatives, thiocarbazide derivatives, aryl hydrazine derivatives, hydrazide derivatives; and oxylamine derivatives.

17. The method according to claim 1, wherein B comprises a functional group selected from the group consisting of any free amino, carboxyl, thiol, alkyl halide, acyl halide, chloroformiate, aryloxycarbonate, hydroxyl, α-haloacetamide, maleimide, azide, carbonyl groups of aldehyde group, carbonates, carboxylic acid activated in situ, carbonyl halides, activated esters, N-hydroxybenzotriazole esters phosphoramidite; H-phosphonates; a phosphor triester or phosphor diester activated in situ; isocyanates; isothiocyanates; NH₂, OH, N₃, O—NH₂, alkyne, alkene, diene, [symbol] β-unsaturated ketone, [symbol] β-unsaturated ester, [symbol] β-unsaturated amide, 3-carboxy-4-nitrophenyldisulfanyl, pyridin-2-yldisulfany, hydrazine derivatives, hydrazine carboxylate derivatives, semicarbazide derivatives, thiosemicarbazide derivatives, carbonic acid dihydrazide derivatives, carbazide derivatives, thiocarbazide derivatives, aryl hydrazine derivatives, hydrazide derivatives; and oxylamine derivatives.

18. The method according to claim 1, wherein L and L′ are selected from the group consisting of a linear or branched divalent organic radical, a cyclic divalent organic radical, and a bond.

19. The method according to claim 18, wherein the linear divalent organic radical includes a multiply functionalized linear or branched alkyl group containing up to 18 carbon atoms.

20. The method according to claim 19, wherein the multiply functionalized alkyl group contains between 2 and 10 carbon atoms.

21. The method according to claim 19, wherein the alkyl chain(s) include(s) at least 1 atom different from carbon.

22. The method according to claim 21, wherein the at least 1 atom different from carbon is selected from the group consisting of N, O, and S.

23. The method according to claim 18, wherein L and L′ are a 5-7 membered ring.

24. The method according to claim 23, wherein the 5-7 membered ring structure contains at least one heteroatom independently selected from N, O, or S.

25. The method according to claim 1, wherein the donor substance has the general formula selected from 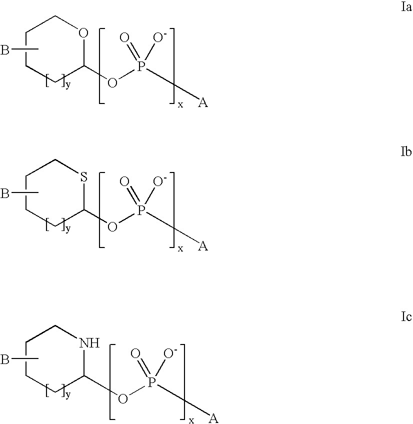
wherein y is 0, 1, or 2;
and optionally wherein any one carbon in the ring structure independently is substituted with hydroxy, hydroxymethyl, N-acylamino, alkyl, alkyloxy, halogen, alkanoyl, aryl, aryloxy, heteroaryl, or heteroaryloxy.

26. The method according to claim 1, wherein the donor substance has the general formula Id 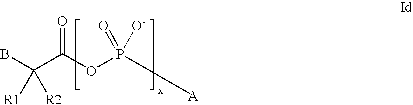
wherein R1 and R2 each independently are selected from hydrogen, alkyl, halogen, alkanoyl, aryl, and heteroaryl.

27. The method according to claim 17, wherein L and L′ are selected from a group selected from 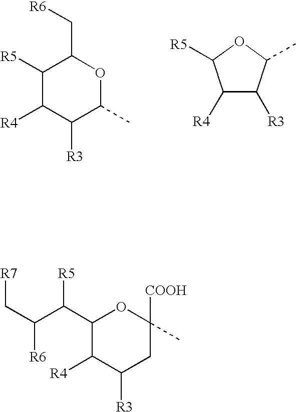
wherein one of R3-R7 is a divalent organic radical attached to B in general Formula I or a valency bond to B in general Formula I, and wherein the remaining R3-R7 each are selected independently from —H, —OH, —CH₂OH, —NH₂, and N-acylamino groups.

28. The method according to claim 1, wherein B is absent, and wherein L′ is selected from the group consisting of

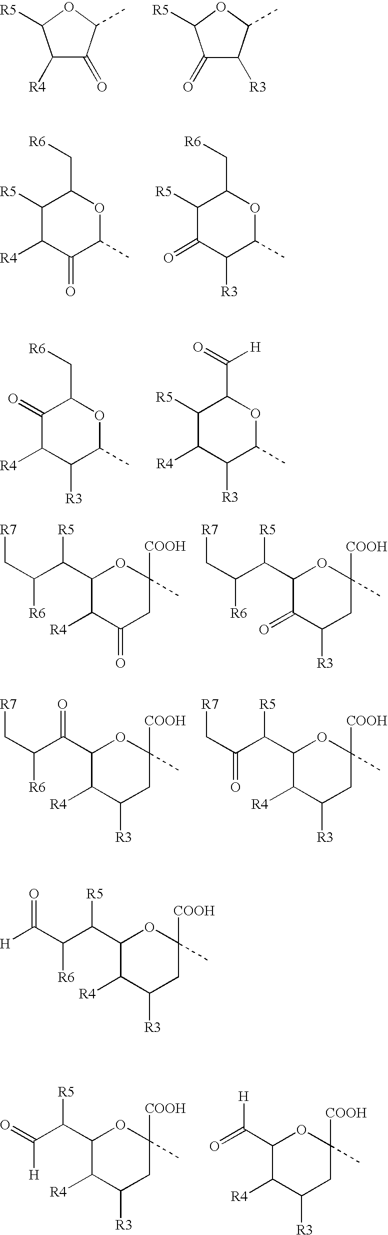

29. The method according to claim 1, wherein the donor substance has the formula selected from the group consisting of 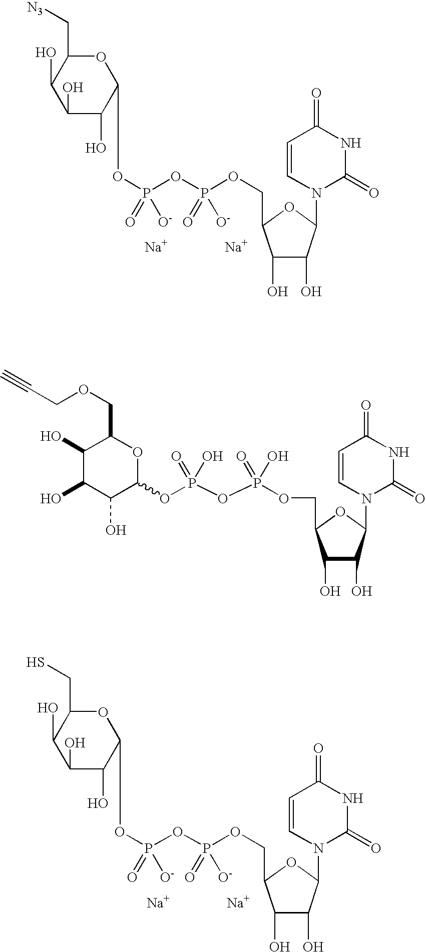
,
, 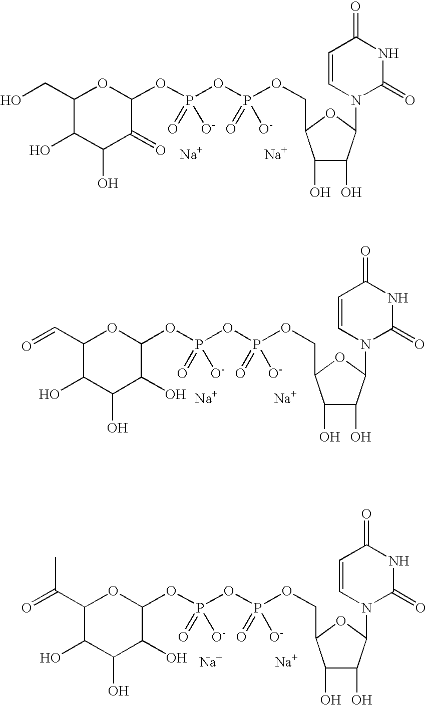
, 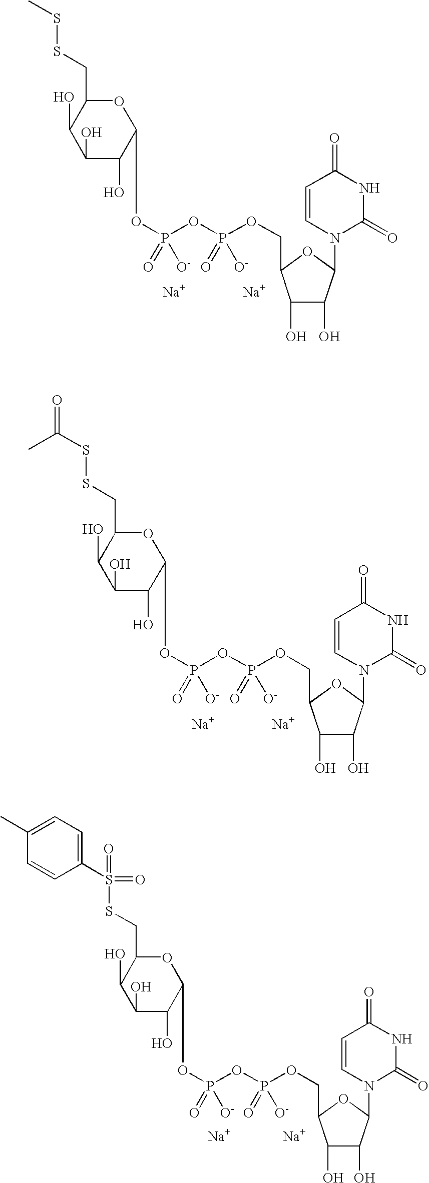 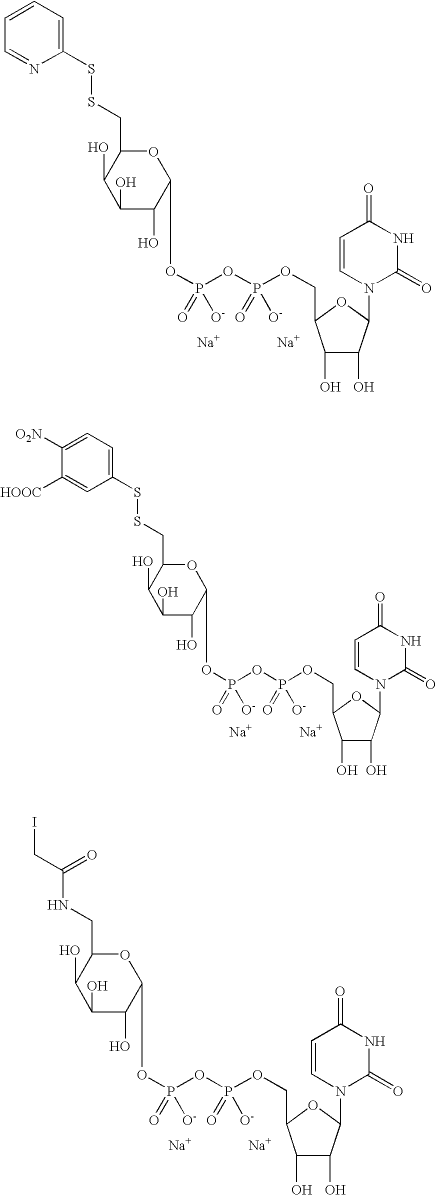 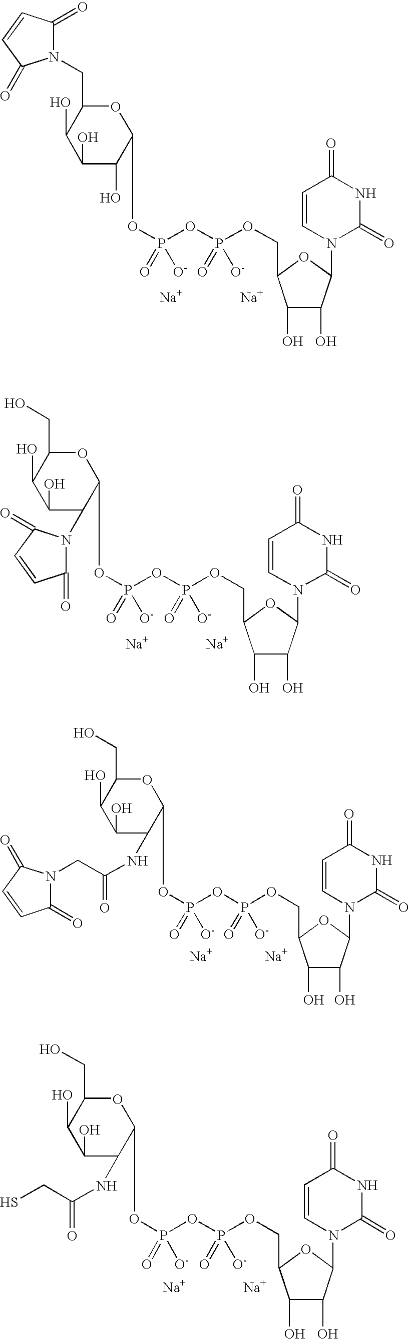 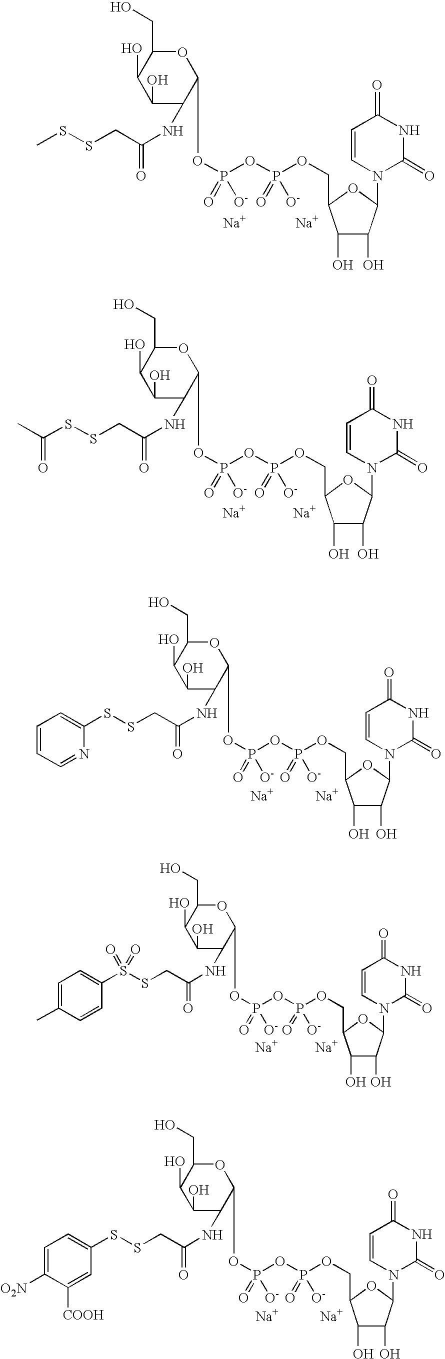
,
,
,
,
,
,
,
,
,
,
,
,
, and
, as well as any stereo isomers or other salts than sodium salts of the compounds selected from said group.

30. The method according to claim 1, wherein M′ is selected from FVII, FVII, FIX, FX, FII, FV, protein C, protein S, tPA, PAI-1, tissue factor, FXI, FXII, FXE, or a sequence variant of any thereof; immunoglobulins; cytokines; alpha-, beta-, and gamma-interferons; colony stimulating factors; platelet derived growth factors; phospholipase-activating protein (PUP); insulin, plant proteins; tumor necrosis factors; soluble forms of tumor necrosis factor receptors; interleukin receptors and soluble forms of interleukin receptors; growth factors; somatomedins; erythropoietin; pigmentary hormones; hypothalamic releasing factors; antidiuretic hormones; prolactin; chorionic gonadotropin; follicle-stimulating hormone; thyroid-stimulating hormone; tissue plasminogen activator; and fusion proteins comprising any of the above mentioned proteins or fragments thereof.

31. A method for the preparation of a modified intermediate of formula B-L-M or L′-M as defined in claim 1, said method comprising steps a and b but omitting step c of the method according to claim 1.

32. A donor substance having the general formula defined in claim 1.

33. A modified intermediate of formula B-L-M or L′-M as defined in claim 1.

34. A donor substance selected from the list consisting of 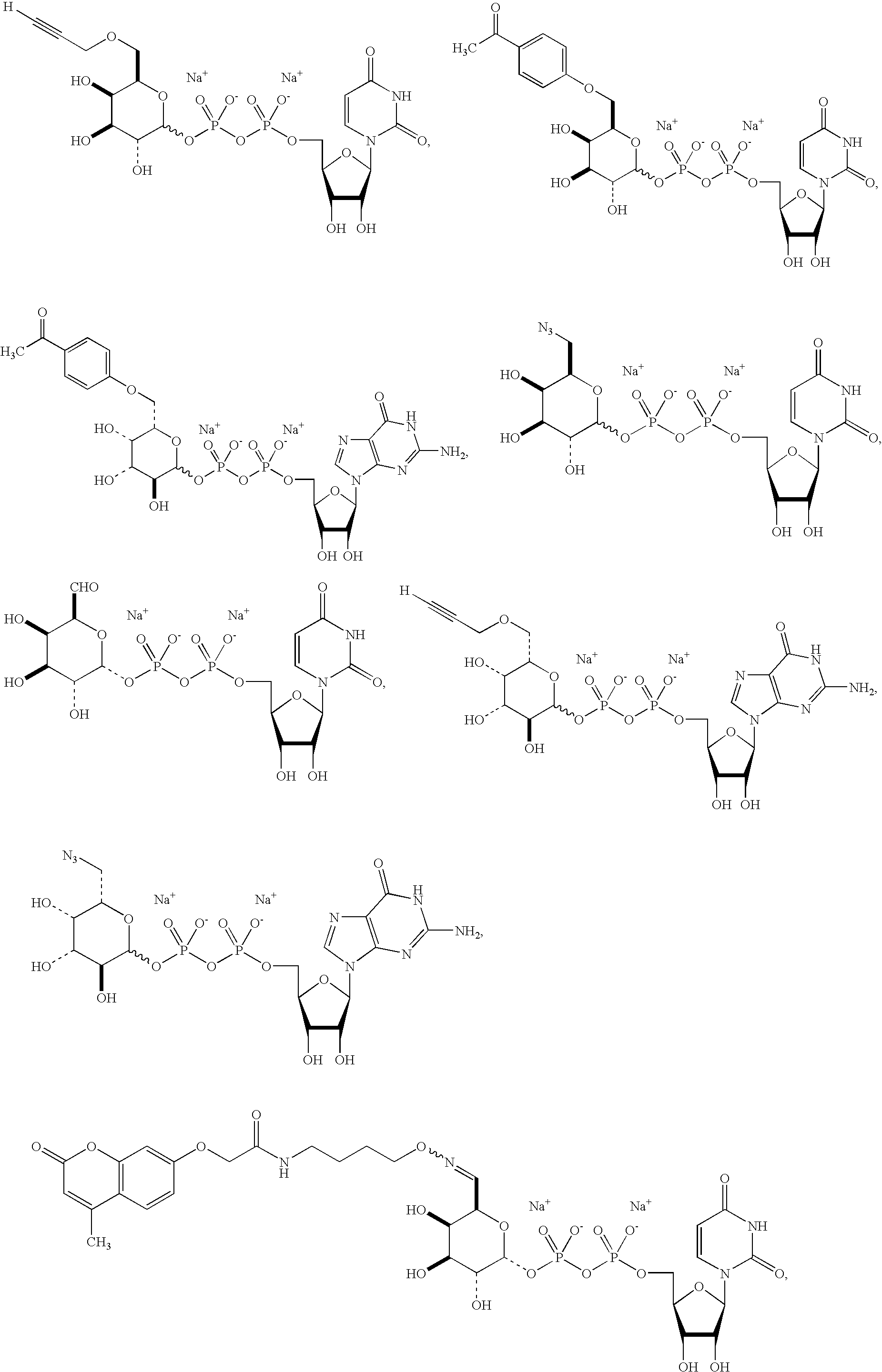 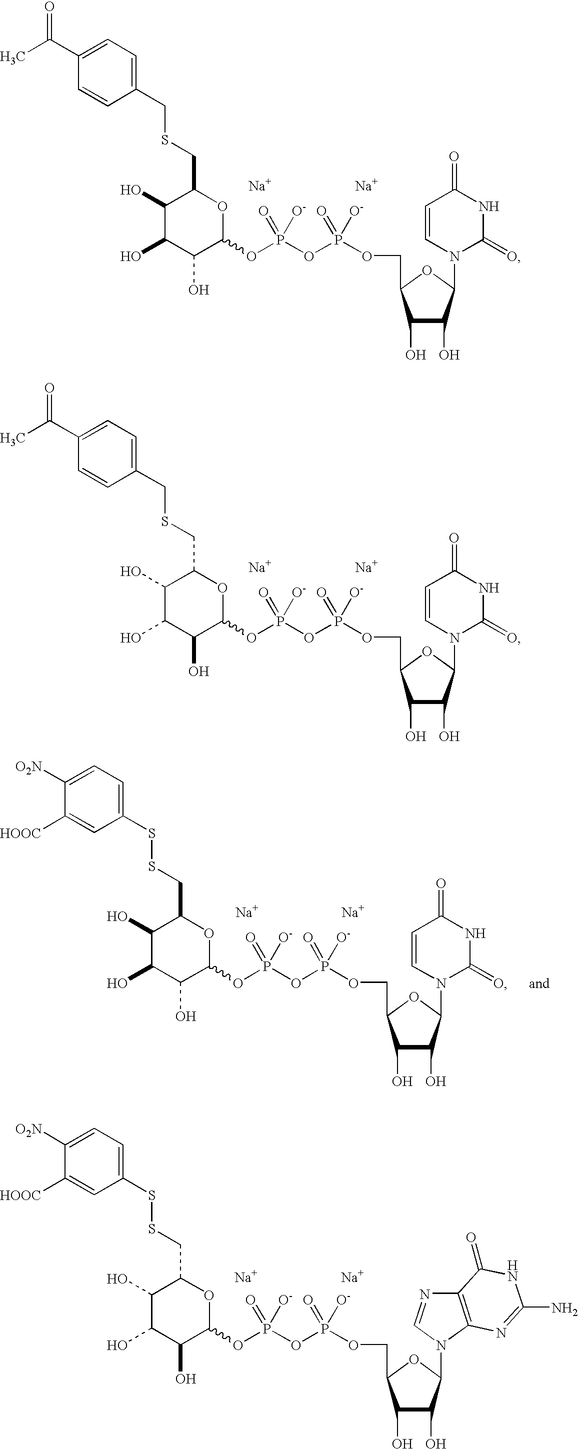
as well as any stereo isomers or other salts than sodium salts of these compounds.

35. A modified analogue P—B′-L-M or β-L-M, obtainable by the method according to claim 1.

36. A pharmaceutical composition comprising a modified analogue P—B′-L-M or β-L-M according to claim 1, in a mixture with a pharmaceutically acceptable carrier, diluent, vehicle or excipient.

37. (canceled)
